SEQUENCE LISTING (105 sequences)

SEQ ID NO: 1 - DNA, 810 bp, Bacillus cereus
atgtttgttactgtcgagaaagatgtgcatatttttgtgcaagatgttaatcccggtcctggtagtaaaactgtgttcttcgtccatggttggcctttaaatcatcaaatgtaccaatatcaactcaacgtgttaccacagcacggtttccgctgcatcgctatggatatacgcggaaatgggcagtctgataaaccatggactggttacacatacgatcgtttagcagacgatatcgccatcgttttagaagcacttcaagtcgaaaatgctacattagtcggtttctcagtaggcggtgccctttctattcggtacatgtctcgctataatggtcagcgtatttctaaacttgtattagtcgatgccgtttccccctctttcgttaaaaatcaagaatctccctatggtgtcccaaaagaacaagcagatactctcattaatcaaatgtatgccaatctccctaaatttttaaatgatgtatctttatcattttttaatagaaacttaggatctgctacgttagaatggttttcttatctcggtatgcagtctgcttcctatgccctcatcaaaatattgcaagcagctgcaaatgaagatgtaacgaaagatttaagtaaaattaacgtgccaacaaaaatattccacggtactcacgatcaacttatcccatacaaaagcgctgaacttacacaaaaacggattaaaaattctcagttacatcctctcaccaatagtggtcacggctccccaatcgatcaagcggatgaattaaataaagaactgataaaatttttacatgcataa

SEQ ID NO: 2 - protein, 269 aa, Bacillus cereus
MFVTVEKDVHIFVQDVNPGPGSKTVFFVHGWPLNHQMYQYQLNVLPQHGFRCIAMDIRGNGQSDKPWTGYTYDRLADDIAIVLEALQVENATLVGFSVGGALSIRYMSRYNGQRISKLVLVDAVSPSFVKNQESPYGVPKEQADTLINQMYANLPKFLNDVSLSFFNRNLGSATLEWFSYLGMQSASYALIKILQAAANEDVTKDLSKINVPTKIFHGTHDQLIPYKSAELTQKRIKNSQLHPLTNSGHGSPIDQADELNKELIKFLHA

SEQ ID NO: 3 - DNA, 825 bp, Bacillus flexus
atgggatattaccttaccgttgatcaaaagggtacgaaaatatatgttgaagacattggaagtggaatgcctatactgtttcttcacggctggccagttaatcataaaatgtttgaatatcagtttaatgagcttccgaaacatgggattcgctgcattgcaatagacttacgcgggtttggaaaatcaagtcgaccgttcaaaggctatacatataactcattagccgacgatgttcgagcagttattcacgccttacaactttcagctctaacgttagttggtttttcaatgggaggagcaatcgccttgcggtatatgagtagacatttagggtttggtgtgaacaaactgattctagctggtgcagctgcccctagctttattaaaagagacggttttccttatggactttctaagggagaagtagatagcatcattcaagcgacgtatcaagatcgaccagctatgctcgccgattttggccagaaattttttaattcaaaaattagctcttcttttagagattggtttcaattactaggtctagaagcttcgggtcacggaacgattaaaacagcaaagtcactgcgtgatgaagatctccgtgaagacttgagtcaaatttttgtaaaagcgtatattttacacggaaagaaagatcaaatttgtccgtttcctttagctgaagccatgcataaaggaattgagggttcaacgttaattccttttgaaaagagcggtcacggccttttttacgatgaacaaaaaaagtttactgaaaccttaaaagacattgtttattctgctgcgaaaagagattga

SEQ ID NO: 4 - protein, 273 aa, Bacillus flexus
GYYLTVDQKGTKIYVEDIGSGMPILFLHGWPVNHKMFEYQFNELPKHGIRCIAIDLRGFGKSSRPFKGYTYNSLADDVRAVIHALQLSALTLVGFSMGGAIALRYMSRHLGFGVNKLILAGAAAPSFIKRDGFPYGLSKGEVDSIIQATYQDRPAMLADFGQKFFNSKISSSFRDWFQLLGLEASGHGTIKTAKSLRDEDLREDLSQIFVKAYILHGKKDQICPFPLAEAMHKGIEGSTLIPFEKSGHGLFYDEQKKFTETLKDIVYSAAKRD

SEQ ID NO: 5 - DNA, 783 bp, Bacillus licheniformis
atgggatattttatcgaggcagaaccaagcgttaatctgtacgttgaagacatcaatcctcaaagcggcaaagccattgtatttttgcacggctggccattaagccatctccagtttgaatatcaatttgacgatctttcgcgaaagggataccgctgtatcggcattgactggcgtggattcgggcgttcggataagccggcgacgggctataattacaaccgtttggctgatgatatccgcgctgtcgttgaggcgcttcaattaaacaacttcacgcttgccggtcattcaaccggcggcgccatagcactccgctatatgtcccgatataggggcgctggggtctcaaagctggttttgatcgatgcggcggcgcctgtgggatttactgaagaaacggcagccaaattgcttcagcaagcgtctaacgatcgtcccaagatgatgcgcgaagtgaccgacacgtttttctttcaatatattacacaaccgttctcagactggttttttcaattgggattgcaggcggcaggctggtcgacaaaagcggtcatcgaaatgctgagagatgagaaactgtatgatgatccggaaaaagtcgctgctccagccttaatcattcacggtattcaagacaaggcgattccgtttgcacaagcaaaggagctcagccgcaaaatcagaaattcatatcttgttccgtttcaatacagcggacacggtccattctgggaggaacgcaaaatgttcaatcggattttaacgcaatttatcggttga

SEQ ID NO: 6 - protein, 260 aa, Bacillus licheniformis
MGYFIEAEPSVNLYVEDINPQSGKAIVFLHGWPLSHLQFEYQFDDLSRKGYRCIGIDWRGFGRSDKPATGYNYNRLADDIRAVVEALQLNNFTLAGHSTGGAIALRYMSRYRGAGVSKLVLIDAAAPVGFTEETAAKLLQQASNDRPKMMREVTDTFFFQYITQPFSDWFFQLGLQAAGWSTKAVIEMLRDEKLYDDPEKVAAPALIIHGIQDKAIPFAQAKELSRKIRNSYLVPFQYSGHGPFWEERKMFNRILTQFIG

SEQ ID NO: 7 - DNA, 795 bp, Rhodococcus ruber
atgcgggacgtcgatctgtactgcgaggacaccggcggtgagggcgagcccatcctgttcctgcacgggttcttcttcgacgggcgaatgtacgaggcgcagatcgcggcgctgcgggatcggtaccgctgcctcaccctcgacttccggggacagggccgatccgggcacgcccggaccggattccagctcgagcagctcaccgccgacgtgctcaccgtgatccggcggctcgacctcgcacccgcgcatctggtggggctgtcgatgggcggcttcgtcggcatgcgcatcggcgcgcgggaaccggacctgttgcgctcgccgaccctgctcaacaccggcgcgacgccgcatccgcccgtgaggtacccgctgcaccttgccctcacggccgtggcccggcttctcgggccgtcgccggccctcgtgctgcgcggcatcgagaaggaactctacggcgaaccgttccgcaccgaccccggccgggaggccgagcgtgaggtgtggcgcaggcggtgggcgacggccgaccggtcgtcactggcgaacacgatgctcggcatgatggtccgccccgacatccgggacgagctgcccgcggtgagcgtgccgacgctgatcgtcgcgggacaactggacgtcgagatgccgccgcgtctcgcccgcgagatgcacgcgctgatcccgggctcgcgactggtggaactcgacggggtgggccacagttcgacgctggaggacccggcccgggtcaccgggatcctggagagcttcctcgccgagatcggctga

SEQ ID NO: 8 - protein, 264 aa, Rhodococcus ruber
MRDVDLYCEDTGGEGEPILFLHGFFFDGRMYEAQIAALRDRYRCLTLDFRGQGRSGHARTGFQLEQLTADVLTVIRRLDLAPAHLVGLSMGGFVGMRIGAREPDLLRSPTLLNTGATPHPPVRYPLHLALTAVARLLGPSPALVLRGIEKELYGEPFRTDPGREAEREVWRRRWATADRSSLANTMLGMMVRPDIRDELPAVSVPTLIVAGQLDVEMPPRLAREMHALIPGSRLVELDGVGHSSTLEDPARVTGILESFLAEIG

SEQ ID NO: 9 - DNA, 83 bp, Artificial Sequence
  oPlastBug-121 oligonucleotide
acaattcctctagaaataattttgtttaactttaagaaggagatatatccatggcttacgaattaccacaattaccttatgca

SEQ ID NO: 10 - DNA, 90 bp, Artificial Sequence
  oPlastBug-121 oligonucleotide
tagcagccggatcaagctgggatttaggtgacactatagaatactcttatgcatgtaaaaattttatcagttctttatttaattcatccg

SEQ ID NO: 11 - DNA, 72 bp, Artificial Sequence
  oPlastBug-232 oligonucleotide
tagaaataattttgtttaactttaagaaggagatatatccatgggatattaccttaccgttgatcaaaaggg

SEQ ID NO: 12 - DNA, 73 bp, Artificial Sequence
  oPlastBug-233 oligonucleotide
caagctgggatttaggtgacactatagaatactcaagctttcaatctcttttcgcagcagaataaacaatgtc

SEQ ID NO: 13 - DNA, 70 bp, Artificial Sequence
  oPlastBug-234 oligonucleotide
tagaaataattttgtttaactttaagaaggagatatatccatgggatattttatcgaggcagaaccaagc

SEQ ID NO: 14 - DNA, 74 bp, Artificial Sequence
  oPlastBug-235 oligonucleotide
caagctgggatttaggtgacactatagaatactcaagctttcaaccgataaattgcgttaaaatccgattgaac

SEQ ID NO: 15 - DNA, 63 bp, Artificial Sequence
  oPlastBug-236 oligonucleotide
tagaaataattttgtttaactttaagaaggagatatatccatgcgggacgtcgatctgtactg

SEQ ID NO: 16 - DNA, 60 bp, Artificial Sequence
  oPlastBug-237 oligonucleotide
caagctgggatttaggtgacactatagaatactcaagctttcagccgatctcggcgagga

SEQ ID NO: 17 - DNA, 810 bp, Artificial Sequence
  Bacillus cereus chloroperoxidase nucleotide sequence codon optimised to Yarrowia lipolytica
atgttcgtgactgtcgagaaggacgtgcacatttttgtccaggacgtgaaccccggacctggctctaagaccgtgttcttcgtgcatggttggcccctgaaccatcagatgtaccagtaccagctgaacgtcctgcctgagcatggattccgatgcattgccatggacattcgaggaaacggccagtctgacaagccctggactggttacacctacgatcgactcgccgacgatattgctatcgtgctggaggctctgcaggtggagaacgctaccctcgtgggattctctgtgggcggagccctctctatccgatacatgtcccgatacaacggtcgacgaatctctaagctcgccctgatcgacgctgtgtctccttccttcgtgaagaaccaggagtctccctacggagttcctaaggagcaggctgacgccctgatcaaccagatgtacgccaacctgcctaagttcctgaacgacgtttctctgtccttcttcaaccgaaacctcggagccgctaccctggagtggttttcctacctgggcatgcagtctgcttcctacgccctgattaagatcctgcaggctgccgctaacgaggatgtgaccaaggacctgaacaagatcaacgttcctaccaagattttccacggagttcatgaccagctgatcccttacaagtctgccgagctgacccagcagcgaatcaagaactctcagctgcacgccctgactaactccggacatggttcccctattgaccaggccgatgagctgaacaaggagctgatcaagttcctgcacgcctaa

SEQ ID NO: 18 - protein, 277 aa, Achromobacter xylosoxidans
MTSNTYITNDGTQIYYKDWGDGPVVTFSHGWPLNADAWDGQMHYLANNGFRVIAHDRRGHGRSSQPSSGNDMNGYADDLAQLIEALDLKHITMVGHSTGGGEVARYIGRHGSKRVARAVLIGAVPPVMLQSAANPEGLPLEVFDGIRAGVASDRSQFYKELAIPFYGANRPDAKVSQGMLDQFWLWSMQSGQKNAYECVKAFSETDFTEDLKNIDVPTLFMHGEDDQIVPVHDAAKKAARLVKNAKEIYYPGAPHGLTATMQDRINADLLAFIRATA

SEQ ID NO: 19 - protein, 276 aa, Acinetobacter baumannii
MGYVTTKDGVDIFYKDWGPRDAQVLFFHHGWPLSSDDWDTQLLFFLKEGFRVVAHDRRGHGRSSQVWDGHDMDHYADDVAEVVKHLDIKNAIHIGHSTGGGEVVRYIARHGQENVNKAVLVSAVPPLMVKTENNPEGLPKEVFDDLQNQVLTNRAQFFRDLPSGPFYGFNRPDAKPSEGIISNWWRQGMTGSAKAHYDGIVAFSQTDFTEDLKKITVPVLVLHGDDDQIVPYKTSGVKSAELLQNSQLKIYPGFSHGMLTVNHEVINADLLAFIRE

SEQ ID NO: 20 - protein, 276 aa, Acinetobacter pittii
MGYVTTKDGVDIFYKDWGPRDAPVIFFHHGWPLSSDDWDAQMLFFLKEGFRVVAHDRRGHGRSTQVWDGHDMDHYADDVAAVVEHLGVQGAVHVGHSTGGGEVAYYVARYPNDPVAKAVLISAVPPLMVKTENNPDGLPKEVFDDLQNQLFKNRSQFYHDVPAGPFYGFNRPGAKVSEPVVLNWWRQGMMGGAKAHYDGIVAFSQTDFTEALKKIEVPVLILHGEDDQVVPFEISGKKSAELVKNGTLISYPGFPHGMPTTEAETINKDLLAFIRS

SEQ ID NO: 21 - protein, 278 aa, Arthobacter sp
MTEVTAHHGLFKDTNLHVDDTGGTGRPVVLIHGWPLSGEAFSAQVPALQTAGYRPITYDRRGFGHSDKPSKGYTYDTLTEDLHALLEALDLTDVTLVGFSMGGGEVARYFTKYGTERIRSIVFASAVPPYMMQGDDNPDGPLTKDAAAQMTEDLTKDEDSFYDGFTRDFFSVNGELKVTEQQRQQALALCKQADKKAALACMEAFGTTDFRDDLTNITVPTLILHGDGDDVVPFEGSGARTHAALIGSDLHIITGAPHGCNVSHPDDFNQALLTFLSK

SEQ ID NO: 22 - protein, 279 aa, Acidovorax sp.
MTTSQYITANDGTQLYFKDWGPKNGQVVVFSHGWPLNSDSWESQMQFLAEKGYRVIAHDRRGHGRSGQPWEGNTMDQYADDLDAVLRKLNVKGVVLVGFSTGGGEVARYVGRHGTARVSKVALVSAITPIMGRTTTHPEGVPTEVFESLRKGSLENRSQLYKDIASGPFFGFNRPGAKVSTGLIDSFWTQGMQASSKATYDCINAFFYSDFRDDLKKFNIPTLVVHGTDDQIVPIETTGRAVAKLVPNAKLIEYAGGPHGITDTHKDQLNNDLLEFVKK

SEQ ID NO: 23 - protein, 278 aa, Bacillus aryabhattai
MAKITVGTENEAPIELYYEDHGTGKPVVLIHGWPLSGRSWEYQVPALIEAGHRVITYDRRGFGKSSQPWDGYDYDTFAADLHQLLEHLDLKNVTLVGFSMGGGEVARYVGTYGTDRVEKAVFAGAVPPFLYKSEDHPEGALDDAGIQEFENGVKNDRLAFLDDFTKTFFGAKDGKGLVSEPFRLYNRDIAAAASPKGTLDCIAAFSKTDFRDDLAKFTIPTLIIHGDADEIVPFEYSGKRTHEAISDSKLALIKGGPHGLNATHAHEFNEALLSFLRD

SEQ ID NO: 24 - protein, 278 aa, Bacillus mycoides
MAKITVGTENQAPIEIYYEDHGIGKPVVLIHGWPLSGRSWEYQVPALVEAGYRVITYDRRGFGKSSQPWEGYEYDTLTSDLHQLLEHLELQNVTLVGFSMGGGEVARYIGKYGTNRVEKAVFAGAVPPFLYKSADHPEGVLDDVAIQGFENGVKSDRLAFLDEFTKGFFAAEDRTDLVSEPFRLYNRDIAAGASPKGTLDCIAAFSKTDFRGDLAKVNIPTLVIHGDSDATVPFEYSGKLTHEAIPNSKVALIKGGPHGLNATHAKEFNEALLLFLKD

SEQ ID NO: 25 - protein, 278 aa, Bacillus sp.
MAKFTVGNENHAPIELYYEDQGSGKPVVLIHGWPLSGRSWEYQVPALIEAGYRVITYDRRGFGKSSQPWEGYEYETFASDLHKLLEHLDLQNVTLVGFSMGGGEVARYISTYGTDRIEKAVFAGAVPPYLYKSEDNPEGALDDATIEEFKSGVINDRLAFLDKFTKGFFAAGNQTDLVSEPFRLYNWDIAASASPKGTLDCITAFSKTDFRKDLEKFNIPTLIIHGDSDATVPFELSGKRTYDSIPGSKLALIKGGPHGLNATHPKEFNEALLTFLKD

SEQ ID NO: 26 - protein, 268 aa, Bacillus subtilis
MGHYIKTEEHVTLFVEDIGHGRPIIFLHGWPLNHKMFEYQMNELPKRGFRFIGVDLRGYGQSDRPWEGYDYDTMADDVKAVIYTLQLENAILAGFSMGGAIAIRYMARHEGADVDKLILLSAAAPAFTKRPGYPYGMRKQDIDDMIELFKADRPKTLADLGKQFFEKKVSPELRQWFLNLMLEASSYGTIHSGIALRDEDLRKELAAIKVPTLILHGRKDRIAPFDFAKELKRGIKQSELVPFANSGHGAFYEEKEKINSLIAQFSNS

SEQ ID NO: 27 - protein, 278 aa, Bacillus thuringiensis
MAKITVGTENQEPIEIYYEDYGTGKPVVLIHGWPLSGRSWEYQVPALVEAGYRVITYDRRGFGKSSQPWEGYEYDTFTSDLHQLLEHLELQNVTLVGFSMGGGEVARYISTYGTDRIEKAVFAGAVPPYLYKSEDHPEGALDDATIEAFKSGVINDRLAFLDEFTKGFFAAGNRTDLVSEPFRLYNWDIAASASPKGTLDCITAFSKTDFRKDLEKFNIPTLIIHGDSDATVPYEYSGKLTHEAIPNSKVTLIKGGPHGLNATHAKEFNEALLSFLKD

SEQ ID NO: 28 - protein, 268 aa, Bacillus vallismortis
MGHYVKTEEHVTLFVEDIGHGRPIIFLHGWPLNHKMFEYQMNELPKRGFRFIGIDFRGYGQSDRPWEGYDYDTMADDVKAVIYTLQLENAVLAGFSMGSAIAIRYMARHDGADIDKLILLSAAAPAFTKRPGYPYGMRKQDIDDMIEQFKMDRPKTLDDLGKQFFEKEVSPELRGWFLNLMLEASSYGTIRSGHALRDEDLRKDLVSIKVPALILHGSKDQIAPFDFAKELKRGIKQSELVPFANSGHGAFYEEKEKMNRLIAQFAKS

SEQ ID NO: 29 - protein, 270 aa, Brevibacillus borstelensis
MGYYVAVEQNVNLYVEDLDPGNGKPVLFIHGWPVNHKMYEYQFNQLPQMGYRCIGVDLRGFGKSDRPWRGYSYDRLADDIRVVIDTLELENITLAGFSMGGAIAIRYMARYGADKVSKLALFAAAAPLFTRRPDYPYGMTKEQVNSLIQATYTDRPKMLNDFGDMFFASYVTEGIKDWMHGLGMEASGHATAMCAVSLRDEDLRRDLSAVSVPTAIFHGVQDKVCPYPFAEFMHAGIQGSELVPFLYSGHGLFYDELEKFNRELVSFLAK

SEQ ID NO: 30 - protein, 269 aa, Brevibacillus brevis
MYIEVEKGVTVFVEDLNPGPNSKTIFFVHGWPLNHNMFSYQFNVLPQHGFRCVAMDIRGNGQSDKPWSGYTYDRLADDIYVVLEALQIRDAVLLGFSVGGAISIRYMSRYEGRHISKLALVDAVSPSFVKMPGSPYGVSKEQAEGLISQMYANMPELLNTVSLQFFNRNLGTATLQWFVKMGLDSASYALIKIMQAASRENVINDLSQIRVPTGIFHGIHDQLIPFKSAELTQQQIKGSKLFPFDNSGHGLPICQADDFNKKLMEFIQS

SEQ ID NO: 31 - protein, 272 aa, Brevibacillus sp.
MTQRLTTASGHYVPVGGNVTLYVEDVGEGKPVVFIHGLPVGQAMYEYQRNVLPFKGIRYIGIDLRGFGKSDRPFNGYSYNQFADDIRRVFEQLEIENATLVGFSMGGAVAVRYMARHQGYGVSKLVLASAATPLFTQRPDYPYGLPVEVVESLVEAAQQDRPQLIEGFNSSLFHQKPSQAFVDWYNRMSLETDPIALAMSLKSLAYEDSREELAQIHVPVRIFQGKHDTMSFEPQINGFPDAKVIVFEESGHGLVFEEKDKFNIEILKFVEA

SEQ ID NO: 32 - protein, 278 aa, Citrobacter amalonaticus
MSQYDKITLKDGSYLYFKDWGDRSGQPIVFSHGWPLTADAFEDQMLFLGSKGYRVIAHDRRGHGRSAQPWEGHTMDQYADDLAELTAHLNLQNAVHVGHSTGGGEVARYIGRHGTQRVAKAVLIGAVTPIMVKTDFNPNGVPKEVFDGIREGVVNDRAAFFYELTAAFYGYNRPGAKESKAVRESFVEQGLQGSIKALYDCIKAFSETDLREDLRKMTIPTLVIHGDDDQIVPFETCGKVAAEILPDSQLKVYAGGSHGICTTHKQQINQDLLNFIQS

SEQ ID NO: 33 - protein, 279 aa, Comamonas sp.
MPYLDISQSNSRPLKLHYQDFGSGPPVVLIHGWPLSARTWEPQIADLVDAGFRVIAYDRRGFGASSQPWGGYDYDSLARDLNELITELELEKVCLVGFSMGGGEVARYIGTYGTAFIRCAVFAAAVPPFLLKCETNPQGAVSEADVAKKEQAVLQDRLSFLDEFTQKFFSNAPDSLLVSEAQREYARTIAAFASAKATHACIGSFSRTDFRDNLQKIDVPTLIIHGDSDRIVPLEVSGARTHDAVRGSHIHIIKNGPHGCNLSHAREFNRALIDFIKAH

SEQ ID NO: 34 - protein, 329 aa, Cupriavidus necator
MQIEHTQEQGHNIDASRRQLLVGSTVGAAAASVASLAALTPTAAEAAPASSRATNQGHYVTTKDGTRIFYKDWGTGSPVVFSHGWPLNADAWDAQMLFLVQKGFRVIAHDRRGHGRSDQPAQGNNMDTYADDLAALLDALDIKGATLVGHSTGGGEVAHYIGRHGTKRVAKAVLIGAVPPIMLKTAANPNGLPMEVFDGIRKGVAENRSQFYKDLTTPFFGFNRPGAKVSQGTIDAFWAQGMTGGLLGQYACIKEFSEVDYTGDLKKITVPTLILHGDDDQIVPIDNSARLSAKIVKNATLKVYPGASHGMCVINADQVNADLLAFLNA

SEQ ID NO: 35 - protein, 273 aa, Enterobacter asburiae
MATFKAKDGTQIYYKDCGAGKPVLFSHGWPLDGDMWDSQLNYLAERGFRAIAFDRRGFGRSDQPWNGYDYDTFASDINDLITTLDLQDVTLVGFSMGGGDVTRYINNYGSARVAGLALLGAVTPIFGKSDTFPQGVDQSVFDGIRDGLRKDRAQFISDFATPFYGINAGQTVSAGALTQTLNIALLASLKGTIDCVTAFGETDFRPDMAKIDVPTLVIHGSNDQIVPFESTGKLAAEMIKNATLKVYDNAPHGFALTHQDQLNEDLLAFVKSL

SEQ ID NO: 36 - protein, 273 aa, Enterobacter sp.
MATFKAKDGTQIYYKDCGAGKPVLFSHGWPLDGDMWDSQLNYLAERGFRTIAFDRRGFGRSDQPWNGYDYDTFASDINDLITTLDLQDVTLVGFSMGGGDVTRYINNYGSARVAGLALLGAVTPIFGKSETFPQGVDQSVFDGIRDGLRKDRAQFISDFATPFYGINAGQTVSAGALTQTLNIALLASLKGTIDCVTAFGETDFRPDMAKIDVPTLVIHGSNDQIVPFESTGKLAAEMIKNATLKVYDNAPHGFALTHQDQLNEDLLAFVKSL

SEQ ID NO: 37 - protein, 273 aa, Flavobacterium sp.
MNKFTVKDGTEIYFKDLGAGQPIFFHHGWPLSSDDWDAQMMFFLEKGYRVIAHDRRGHGRSTQTFQGNDMNTYAADVAELVAFLDLKNVIHVGHSTGGGEALRYAAQYGKDRVSKVVLISAITPYMIADENNPNGVPLSVFDDIRYNTLHNRAQFFQDITIPFFGYNREGVTIKKGIQDNWWRQGMMGGIKAQYDCIEVFSETDFTEDLKSITIPILVLHGDDDQIVPYETTAVKAAELLQNGKLIIYPGFSHGMPTTEAATINKDILEFINE

SEQ ID NO: 38 - protein, 278 aa, Klebsiella pneumoniae
MAFVTTQDGVNIYFKDWGPKEAQPIVFHHGWPLSADDWDNQMLFFLAEGFRVIAIDRRGHGRSDQVSEGYDMDHYAADVSAVVEHLDLHNAVHVGHSTGGGQVARYVARYGQPQGRVAKAVLISAVPPLMVKTEQNPGGTPIEVFDGFRKALAANRAQFYLDVASGPFYGFNRDGAEISQGTIQNWWRQGMIGSTKAHYEGIKAFSETDQTEDLKSITLPVLVMQGDDDQVVPYKNAAILQDKLLPNSQLKIYPGFPHGMHTSHANTINADLLAFIRA

SEQ ID NO: 39 - protein, 278 aa, Leucobacter sp.
MTEITAHHGLFKNTNLHVDDPGGSGRPVVLIHGWPLSGESFAAQVPALTEAGYRAITYDRRGFGRSDKPLTGYTYDTLAEDLHTLLEQLDLRDATLVGFSMGGGEVARYFAKYGPERVRSAVFASAVPPYLLKTGDNPDGPLEKSQAAKMTAELTANEDRFYDGFLTEFFSVDGVLQVGEEQRLRAFEMAKQANKAAALACMAAFATTDFRDDLPAITVPTLVLHGDGDATVPFEGSGARTHAAIPGSELVILRGAPHGANVSHAGEWNEALLDFLER

SEQ ID NO: 40 - protein, 259 aa, Lysinibacillus xylanilyticus
MGYFVTVEPGVNVFIEDINPKGNKTILFIHGWPLNHNQFEYQFNFLPTLGYRCIGMDWRGYGKSDKPFSGYSFDRLADDVRMVIETLQLSNITLVGHSTGGAISIRYMARYKGYGVSKLVLVDAASPSSVPKEFTNKIIVDTNHDRPQMLQDQTRIFFFQYISELKSDWFVLMGLQAANWATSAIMVTLRDENVYNDLGQIDVPTLIIHGIHDKVVPFTQAEETNKLIKNSTLVPFQYSGHCAFLEERDRFNQLLSSFA

SEQ ID NO: 41 - protein, 275 aa, Meyerozyma guilliermondii
MTTSSKLTLSDGTELYYKDWGTGEPIVFSHGWPLTSDAFEDQMIYLASKGYRVVAHDRRGHGRSSQPWNGHNMNQYADDLAELVKHLDLKNAVHVGHSTGGGEVARYISRHGTSRVSKAVLLGAIPPLMLKTDANPSGTPKEVFDGIRANVLKDRSKFFKELALSFYGFKEHNSDTGGLIDSFWLQGMQGSIKALHDCVEAFSETDQHEDLKKMDIPTLVGYGDNDQIVPPGPSSEAAIKLLPQATEKVYPGASHGLCSTHKDQVSKDIYEFIKQ

SEQ ID NO: 42 - protein, 280 aa, Microbacterium paraoxydans
MGYITVGTENTTPIELYYEDQGSGQPVVLIHGYPLNGHSWERQTRELLAQGYRVITYDRRGFGQSSKVGSGYDYDTFAADLNTVLETLDLRDVVLVGFSMGTGELARYVGRYGHERIAKLAFLASLEPFLVQRDDNPEGVPQEVFDGIEAAAKGDRYAWFTQFYTDFYNLDENLGTRISQQVVDANWNLSVTSAPVAAYAVVPTWIEDFRDDVKAVAAAGKPTLILHGTKDNILPIDATARRFRQAVPEAEYVEVEGAPHGLLWTHADEVNAALTQFLAR

SEQ ID NO: 43 - protein, 281 aa, Micrococcus luteus
MTLMHIPTDHGTIGLNVEEAGHGRPVVLIHGWPLTLDSWKDQVAALSPHYRVITYDRRGFGASEKPAGGYDYDTMTADLHGLLTELDLRDVTLVGFSMGGGEVARYMSRFGADRIHSVVFAGAVPPYLLKTDDNPDGPLTEKQAAEMRAGLEKDRESFFDDFTTTFFSTPKLAGLTSSLKVTEEQRQEAIRMALQSDQTAALACMDAFGTTDFRRDLPAVTVPALVLHGDADQIVPFKGSGKRTHKAIDGSRVELVKDAPHGFSISHSEEFNRILLEFLAE

SEQ ID NO: 44 - protein, 281 aa, Micrococcus sp.
MTLMQIPTDHGTIGLNVEEAGHGRPVVLIHGWPLTLDSWKDQVAALSPHYRVITYDRRGFGASEKPAGGYDYDTMTADLHGLLTELDLRDVTLVGFSMGGGEVARYMSRFGADRIHSVVFAGAVPPYLLKTDDNPDGPLTEKQAAEMRAGLEKDRESFFDDFTTTFFSTPKLAGLTSSLKVTEEQRQEAIRMALQSDQTAALACMDAFGATDFRRDLPAVTVPALVLHGDADQIVPFKGSGKRTHKAIDGSRVELVKDAPHGFSISHSEEFNRILLEFLAE

SEQ ID NO: 45 - protein, 319 aa, Ochrobactrum intermedium
MTQLSMTATRRSIMSGTIALAAAGTTLGSAPAQASNPLKTRSGDNRVVTKDGVAIFYKDWGPKDAQPIVFHHGWPLSGDDWDNQMLFFLGKGFRVIAHDRRGHGRSAQVSDGHDMDHYAADVFAVIEHLGIHNAIHIGHSTGGGEATRYTAKYGKGRVSKLVLIGAVPPIMVKTPSNPGGLPIEVFDGFRSALAANRSEFYRDLASGPFYGFNRPGAKISEPLIQNWWRQGMAGAANAHYLGIKAFSETEFTEDLRSISVPTLVMHGDDDQVVPIADSAPLSAKLLQNAELKIYKGFPHGMATVQADIINADLLEFIKK

SEQ ID NO: 46 - protein, 260 aa, Paenibacillus sp.
MGYFVNVESGVNLYVEDINPGGSKTILFLHGWPLNHNQFEYQFNVLPSMGYRCIGIDWRGFGKSDKPMNGYNYNRLADDIRAVVGALQLNNFTLAGHSTGGAIAIRYMSRYNGYGVSKLILIDAAAPTGFTAETASTFLAEALNDRPKMIRRVTETFFFQYISDPFSNWFYQMGLQAAGWSTAAIIVTLRDEKLDADLPKIRVPTLITHGIHDKVIPFAQAQVLNQQINNSKLVPFQYSGHGPFWEERDKFNQLLAQFIG

SEQ ID NO: 47 - protein, 274 aa, Pantoea sp.
MSTFKAKDGVNLYYKDWGKGQPVLFSHGWPLDADMWDSQLNFLAERGYRAIAFDRRGFGRSEQPWEGYDYDTFADDIHALIEHLQLDDITLVGFSMGGGDVSRYIGRYGTAKVKGLVLLGAVTPIFGKTDEHPEGVEKAVFDGIKDGLRKDRAQFIKDFATPFYGANAGQTISDGVMTQTLNIALLASLKGTIDCVTAFAENDFRADIAKVDVPTLVIHGSNDQIVPFEATGKLVHEMIEGSELKVYENGPHGFAVTHQDQLNDDLLAFLQGNK

SEQ ID NO: 48 - protein, 272 aa, Pseudomonas aeruginosa
MSTFVTRDGTSIYFKDWGTGKPVLFSHGWPLNADMWDTQMEFLASRGYRAIAFDRRGFGRSSQPWNGYDYDTFADDIAQLIEHLDLREVTLVGFSMGGGDVSRYIGRYGTERVAGLVLLGAVTPVFGKRDDNPEGVDLSVFEGIVAGLRADRAQFIADFAAPFYGINHGQQVSQGVQTQTLNIALMASLKATLDCVTAFSETDFRSDMAKVDVPTLVIHGDDDQIVPFETTGKQAAAQIRGAELKVYAGAPHGFAVTHAQQLNEDLLAFLSR

SEQ ID NO: 49 - protein, 272 aa, Pseudomonas azotoformans
MSTFVAKDGTQIYFKDWGSGKPVLFSHGWPLDADMWEYQMEYLSSRGFRTIAFDRRGFGRSDQPWTGNDYNTFADDIAQLIEHLDLKDVTLVGFSMGGGDVARYIARHGSAHVAGLVLLGAVTPLFGQKPDYPQGVPTEVFDGIKAGLLKDRAQFISDFNTPFFGINQGQKVSDGVLTQTLQVAMLASLKSTVDCVTAFSETDFRPDMTKIDVPTLVIHGDGDQIVPFETTGKVAAASIKGAELKVYKDAPHGFAVTHAQQLNEDLLAFLNR

SEQ ID NO: 50 - protein, 272 aa, Pseudomonas chlororaphis
MSTFTTQDGTEIYYKDWGSGKPVLFSHGWPLDADMWEYQMEYLSSRGYRTIAFDRRGFGRSDQPWTGYDYDTFADDIAQLINHLDLRDVTLVGFSMGGGDVSRYIARHGSERVAGLVLLGAVTPLFGKKADFPQGVDTSVFDGIKAGLLQDRAQFIADFAAPFYGTNQGQKVSDGVLTQTLNIALLASLKGTVDCVTAFSETDFRPDMAKIDVPTLVIHGDGDQIVPFETTGKQAAAQIKGAELKVYAGAPHGFAVTHAQALNEDLLAFLNR

SEQ ID NO: 51 - protein, 272 aa, Pseudomonas fluorencens
MSTFTTQDGTEIYFKDWGSGKPVLFSHGWPLDADMWEYQMEYLSSRGYRTIAFDRRGFGRSDQPWTGYDYDTFADDIAQLINHLDLRDVTLVGFSMGGGDVSRYIARHGSERVAGLVLLGAVTPLFGKKADFPQGVDTAVFDGIKAGLLQDRAQFIADFAAPFYGTNQGQQVSDGVLTQTLNVALLASLKGTVDCVTAFSETDFRPDMAKIDVPTLVIHGDGDQIVPFETTGKQAAAQIKGAELKVYAGAPHGFAVTHAQALNEDLLAFLKR

SEQ ID NO: 52 - protein, 272 aa, Pseudomonas monteilii
MSTFVTRDGTSIYFKDWGSGKPVLFSHGWPLDADMWDSQMEYLASRGYRAIAFDRRGFGRSSQPWSGYDYDTFADDIAQLIEHLDLRDVTLVGFSMGGGDVSRYIARHGSERVAGLVLLGAVTPVFGKRDDNPEGVDLSVFEGICTGLRADRAQFIADFATPFYGINHGQQVSQGVQTQTLNIALMASIKGTLDCVTAFSETDFRPDMAKIDVPTLVIHGDDDQIVPFESTGKRAAELIRGAELKVYAGAPHGFAVTHAQQLNQDLLAFLQK

SEQ ID NO: 53 - protein, 275 aa, Pseudomonas protegens
MSTFITRDGTELYYKDWGSGQPVVFSHGWPLSADSWESQMLFLAEHGYRVIAHDRRGHGRSSQPWDGNDMDTYADDLAQLIEHLDLQDAVLLGFSTGGGEVARYIGRHGTGRLAKAGLISAVPPLMLKTATNPGGLPLEVFDGIRAGAVADRSQLYKDIASGPFFGFNRPGAKVSQGMIDSFWLQGMQAGHKNAYDCIKAFSETDFTEDLKRFDIPTLVVHGDDDQIVPIDAAGRASAKLIANARLLVYPGAPHGLTDTHKERLNHDLLTFIQQS

SEQ ID NO: 54 - protein, 276 aa, Pseudomonas putida
MSYVTTKDGVQIFYKDWGPRDAQVIHFHHGWPLSADDWDAQMLFFLGQGFRVVAHDRRGHGRSSQVWDGHDMDHYADDVAAVVEHLGIQGAVHVGHSTGGGEVVRYMARYPGDKVAKAVLIAAVPPLMVQTPANPGGLPKSVFDDFQAQVATNRAQFYRDVPSGPFYGYNKPGVTPSEGVIANWWRQGMIGSAKAHYDGIVAFSQTDFTDDLKGISQPVLVMHGDADQIVPYENAGPLSAKLLPNGTLKTYAGYPHGMPTTHADVINADLLAFIRS

SEQ ID NO: 55 - protein, 276 aa, Pseudomonas sp.
MSYVTTKDGVQIFYKDWGPRDAPVIHFHHGWPLSADDWDAQLLFFLAQGFRVVAHDRRGHGRSSQVWDGHDMDHYADDVAAVVDHLGIQGAVHVGHSTGGGEVVRYMARYPGDKVAKAVLIAAVPPLMVQTPANPGGLPKSVFDGFQAQVASNRAQFYRDVPTGPFYGYNRPGAKPLEGIIGNWWRQGMIGSAKAHYDGIVAFSQTDFTEDLKRITQPVLVMHGDDDQIVPYENAGPLSAKLLPNATLKTYKRYPHGMPTTHADVINADLLSFIRS

SEQ ID NO: 56 - protein, 274 aa, Pseudomonas stutzeri
MGTITTRDGTQIYYKDWGSGPVVVLSHGWPLNADSWESQMFFLASNGYRVIAHDRRGHGRSSQPWHGNEMDTYADDLAQLLDHLDVRDASVFGFSTGGGEVARYIGRHGTSRIARAGLISAVPPLMVKTENNPGGLPISVFDGIRAASIADRSQLYKDVASGPFFGYNRAGAKPSQGVIDAFWMQGMMAGHKNAYDCIKAFSETDFTEDLKKFDVPTLILHGDDDQIVPVDAAARASAELVPNAKLVIYPGAPHGITDTHKDKLNEDMLAFLRS

SEQ ID NO: 57 - protein, 272 aa, Pseudomonas syringae
MSTFKTKDGTEIYFKDWGTGKPVLFSHGWPLDADMWEYQMEYLSSRGYRTIAFDRRGFGRSDQPWTGYDYDTFADDIAALIQHLDLRDVTLVGFSMGGGDVTRYIARHGTERVAKLALLGAVTPFFMKSDDHPQGVDKSVFDGIKAGLLKDRAQFIADFATPFYGTNQGQTVSDGVMTQTLNIALLASLKGTVDCVTAFSETDFRPDMAKIDVPTLVIHGDGDQIVPFEATGKLAAEMIKGAELKVYSGAPHGFAVTHGQQLNEDLLAFLAE

SEQ ID NO: 58 - protein, 273 aa, Rahnella aquatilis
MSTFNTQDGTQIYFKDWGKGKPVLFSHGWPLDADMWDSQMNFLADRGYRVIAFDRRGFGRSEQPWEGYNYDTFASDINDLIEHLDLHDVTLVGFSMGGGDVTRYISRYGSERVTSLVLLGAVTPLFIKTDDHPQGVDKSVFDGIRAGLLKDRAQFISDFATPFYGTNQGMTVSDGVMTQTLNIALMASLKGTVDCVTAFSETDFRPDMAKITVPTLVIHGSADQIVPFEATGKLAAEMIKNAELKIYEGAPHGFAITHQDQLNDDLLAFLSGK

SEQ ID NO: 59 - protein, 332 aa, Ralstonia sp.
MSTQDTSLPAPHDLSRRKLLVSGASTMAAAAAGALVAGTASSAQAANPKTSHPYSGDHNMSFVTTKDGTRIFYKDWGSGRPVVFSHGWPLSADAWDSQMLFLLQHGYRVIAHDRRGHGRSDQPSKGNNMDTYADDLATLLDTLDIKGATLVGHSTGGGEVAHYIGRHGTKRVAKAVLIGAVPPIMAKTAAYPNGLPIEVFDGIRKGVAENRSQFYKDLTTPFFGFNRPGAKISQGTIDAFWAQGMAGGIHGQYQCIKEFSEVDYTEDLKKIDVPTLILHGDDDQIVPIDNSAKLSAKLVKHATMKIYPGASHGMCVINADQVNADLLAFLSA

SEQ ID NO: 60 - protein, 264 aa, Rhodococcus rhodochrous
MGYIAVGTENSTPIELYYEDHGSGQPVVLIHGYPLSGTSWEKQTAALLEAGYRVITYDRRGFGRSSKPTIGYDYDTFAADLHTVLSTLDLRDVVLVGFSMGTGEVGRYLGTHGSERVAKAAFLGSLEPFLLRTDDNPTGVPREVFDGLRAAVEADRYAFFTDFFRNFFNTDDYLGTRLSEEALRASWQVAAGASPYASVAAQPTWLTDFREDIPKIDVPALIVHGTADNILPIDATAREFAKRLPAADYVEIEGAPHGLLWTHG

SEQ ID NO: 61 - protein, 279 aa, Rhodococcus ruber
MTMPRIPDTRRSYLRVRDVDLYCEDTGGEGEPILFLHGFFFDGRMYEAQIAALRDRYRCLTLDFRGQGRSGHARTGFQLEQLTADVLTVIRRLDLAPAHLVGLSMGGFVGMRIGAREPDLLRSLTLLNTGATPHPPVRYPLHLALTAVARLLGPSPALVLRGIEKELYGEPFRTDPGREAEREVWRRRWATADRSSLANTMLGMMVRPDIRDELPAVSVPTLIVAGQLDVEMPPRLAREMHALIPGSRLVELDGVGHSSTLEDPARVTGILESFLAEIG

SEQ ID NO: 62 - protein, 264 aa, Rhodococcus sp.
MRDVDLYCEDTGGAGEPILFLHGFFFDGRMYEAQIAALRDRYRCLTLDFRGQGRSGHARGGYQLEQLTADVLTVIRRLDLAPAHLVGLSMGGFVGMRIGAREPDQVRSLTLLNTGATPHPPAKYPLHLALTAVARLLGPSPALVLRGIEKELYGAPFRTDPTREPERQAWRRRWAQADRSSLANTMLGMMVRPDIRDELPAVSVPTLIVAGQLDVEMPPHLSREMHALIPGSRLVELDGVGHSSTLEDPARVTGILERFLAEVG

SEQ ID NO: 63 - protein, 276 aa, Serratia marcescens
MGYVTTSDGVEIFYKDWGPKNGKVIYFHHGWPLSSDDWDAQMLFFVNKGFRVVAHDRRGHGRSSQVWEGHDMDHYADDVAAVVKHLGVQGAMHVGHSTGGGEVVRYIVRHGEDKVSKAVLISAVPPLMVKTDSNPQGTPKSVFDDFQAQLAANRAQFYYDVPAGPFYGYNRPGAKPSEAIIWNWWRQGMMGGAKAHYDGIVAFSQTDFTEDLKKIAIPVLVIHGDNDQVVPYQDSGALSAKLVQNGTLHTYKGAPHGIPTTHADQVNADLLAFVNS

SEQ ID NO: 64 - protein, 271 aa, Staphylococcus sp.
MKHRIQVSDEVALNVDDFGSGQPLVFLHGWPANNTMFEYQADFVVENGYRYIGIDHRGFGLSDRAADGYDYDTIADDIQKVVDELNLKDIIVVGFSVGGALALKYGVKHNSSNLKKMVLLGPAGPSFVQRDGYPYGLKPEDVTALIEQINDDQPKAIGDFGEDSFFNSDIEVSDEYKQYFNAMTLTSSLIGTVKLAESLRDEDLREEIKSLDLPVYGIHGTADHVCPIEFSEYLEKEAPDYTLSKVEGAGHALFFEKKDEVNRLLLEALKK

SEQ ID NO: 65 - protein, 270 aa, Stenotrophomonas maltophilia
MTASRVRLHVEDSGGTGRPVVLIHGWPLSAESWKAQVPVLRDAGYRVIAYDRRGFGRSDKPADGYEYDSLAADLAGVIEDLDLKDVSLVGFSMGGGEVARYIANHGQDRLHSVVFAAAVPPYLLKTSDNPDGPLTKEAADEMAGALAKDREAFFDDFTRKFFSANGKLKVTEAQRLEAVALAHQSDQTAALGCQKAFGTTDFREDLKKVTVPTLVLHGDSDGTVPFEGSGKRTHEAIAGSEVVVLKDAPHGCNVSHADDFNLALLNFLKK

SEQ ID NO: 66 - protein, 274 aa, Stenotrophomonas panacihumi
MSHFVTVKDGAQIFYKDWGTGQPVVFAHGWPLSADAWDPQMLFMGQHGYRVVAHDRRSHGRSSQTWDGNNMDTYADDLAAVIEKLDLKDIILVGHSTGGGEIAHYVGRHGTKRVAKMVLVGAVPPLMLKTPANPNGTPLSVFDGIRAGTAGDRSQFFQDLATPFFGANRAGNKVTQGMRDSFWLQGMAGGVKGQYDCIHEFSEVDYTPDLKKIDVPALVVHGDDDQIVPIDASARLSSKLIKGAQLKIYPGAPHGLTVTHAGQFNQDLLAFAKA

SEQ ID NO: 67 - protein, 274 aa, Stenotrophomonas sp.
MSSYHTVKDGTRIFYKDWGHGQPVVFAHGWPLSSDAWDPQMLFMGQHGYRVIAHDRRSHGRSTQTWDGNSMDTYADDLASLIEALDLKDAILVGHSTGGGEIAHYIGRHGSKRVAKAVLVGAVPPQMVVSATNPGGLPMSVFDGIRAGVAADRSQFYQDLTTPFFGANRDGHKVSQGMRDAFWLQGMQGGHKGQYDCIREFSEVDYTQDLKKIDVPTLVVHGDDDQIVPIDASGRLSAKIVANATLKVYPGAPHGLTVTHADQFNQDLLAFAKA

SEQ ID NO: 68 - protein, 279 aa, Streptomyces griseus
MGYIAVGSENSTPIELYYEDQGAGQPVVLIHGYPLNGHSWERQTRDLLDAGYRVITYDRRGFGRSSKAGTGYDYDTFSADLNSLLESLDLRDVVLVGFSMGTGELARYVARYGHERVAKLAFLAALEPFLVARDDNPEGVPQEVFDGIAAAAKGDRYAWYTQFFADFYNLDDNLGSRISQEAVTASWNVATSSAPVAAYAVVSSWIEDFRADVQAVRDSGKPALILHGTADRILPIDATARRFRRKLPEAQYVEIEGAPHGLLWTHAEEVNAALRTFLG

SEQ ID NO: 69 - protein, 275 aa, Streptomyces sp.
MPVCTTRDGVEIFYKDWGRGRPVVFIHGWPLNGDAWQDQLKAVADAGYRGIAHDRRGHGRSTPVYDGYDFDTFADDLNDLLTHLDLQDVTLVAHSMGGGELARYIGRHGTGRVRSAVLLSAVTPLMLQGPDNPEGVPQSVLDDIKAGILKERSQFWKDTAETFFSADRPDSKVTQGNKDAFWYMGMAQTIEGAVACVDAFGTTPFHDDLRKFDVPTLVVHGDDDRVVPIDATARKTVQIVPDAELKVYEGASHGLAMVDGDKQRFNQDLLDFLSR

SEQ ID NO: 70 - protein, 279 aa, Bacillus circulans
MPKLTVDTVNNEPVKIYYEDHGSGKPIVLIHGWPLSSRSWEAQVPALIDAGYRVITYDRRGFGQSSQPYDGYDYDILAKDLHTLIEHLELKEVTLVGFSMGGGEVVRYISIYGEERIEKAVLAAAVPPYMYKSADHPEGALDDATIAEFENGVKADRIAFLEEFTHNFFTAGDNKDLVSEPLRLYNRDIAAFASPKGTLDCIDAFARTDFRNDLASITVPTLVIHGDADGVVPLEASGKLAHKMLANSELVVVEGAPHGFNATHPDKFNEELIQFLAKY

SEQ ID NO: 71 - protein, 276 aa, Aspergillus acidus
MPHLKLRDGAELFYKDWGNPRGPIVTFSHGWPLSSDNWEQQMMFLGEHGYRVIAHDRRGHGRSTQTWDGNNMDTFFDDLEELFAHLKVQDAVMVGHSHGGGEVAHYLGKHGTKRFKKAVLVGAVPPLMVKSSGNPEGTDKSVFDSFRQAMRQDRAQFFLDVPSGPFFGFNRPGAKKSEGQIRSWWQQGMNTSFKSAYDSIKDFSETDFSEDLKKINIPVLVLHGDDDQVVPIEAAGLKSAKLLPQGKLKVYKGGSHAIHNINVDEVNQDLLDFIKS

SEQ ID NO: 72 - protein, 276 aa, Aspergillus brasiliensis
MPHLKLRDGAELFYKDWGNPRGPIITFSHGWPLSSDNWENQMMYFGEQGYRVIAHDRRGHGRSTQTWDGNNMDTFVDDLEELFAHLNVHDAVMVGHSHGGGEVTHYLGKHGTRRFKKAVLVGAVPPLMVKSSANPEGTDKSVFDSFRQSMREDRAQFFLDVPSGPFFGFNRPHAKKSEGQIRSWWQQGMNTSFKSAYDSIKDFSETDFTEDLKKINIPVLVLHGDDDQVVPIEAAGLKAAKLLPQGKLKVYKGGSHAIHNINADEVNRDLLEFIKS

SEQ ID NO: 73 - protein, 264 aa, Aspergillus clavatus
MPYLEVHDRHICYADSHPQGAPKDGETFVFIHGLGSSQNYYFPILDHLTPSHRCITLDTYGSARSTYTGLPISIASIAADVVGVLDSLNIPKAVVVGHSMGGLTVNLLGAQYSDRIRGVVAIGPTHPSEAIATTMSKRSETVLEAGMEPMSNTIPNAATGSQTSPLAKSFIRELLLGQNPKGYAALCQAVANSPVVDYSTITVPFLLIAGEEDKSAPLDSCKYIFDRVSSEKKKMEVLPKVGHWHCIEAPDQVGKTIVDFVGSW

SEQ ID NO: 74 - protein, 276 aa, Aspergillus flavus
MPYLKLRDGAELFYKDWGNPDGEIVTFSHGWPLSSDNWENQMFFLAERGYRVIAHDRRGHGRSTQTWNGNNMDTFVDDLKELFEHLDIKDAMMVGHSHGGGEVTHFLGKHGTSRVKKAVLVGAVPPLMLKSAANPEGTDQSVFDSFRQAMRKDRAQFFLDVPSGPFFNFNRDGVQKSEGQIRSWWQQGMNTSFKTAYDAIKDFSETDFTEDLKKIDIPVLVLHGDDDQVVPIEASGHKSVKLLRHGKLKVYPGGSHAIHNINIEEVNKDLLDFLQS

SEQ ID NO: 75 - protein, 264 aa, Aspergillus fumigatus
MPCLEVCGHSIHYADSHPNGAPKDGATIVFIHGLGSSQNYYFPILHHLTLKHRCITLDTYGSARSPYTGQAITISTIASDVIAVLDALHIPKAVVVGHSMGGLVVTLLGAQYSDRITTVVAVGPTHPSESLASTMRQRSDTVLEVGMEAMANTVPNAATGSQASPLAKAFIRELLLGQNPNGYATLCQAIAKAPAIDYSRITVPFLLIAGEEDKSASLEGCKYIFDRVSSKTKKMEVLPKVGHWHCIEAPDLVGGMIAGFIERV

SEQ ID NO: 76 - protein, 263 aa, Aspergillus japonicus
MPTVTVNHHEIYYADTHPAGAPAHGSTLIFIHGLGSSQNYYFPILPHLEQHRCITLDTYGSARSPYTGQPVSIASIAADVVGVLDALQVAHAVVVGHSMGGLVVTLLGAQYADRVKAVVAIGPTHPSDQLASVMTQRSATVANSGMEPMANTIPSGATGPRCSPLAQALIRELILGQNPQGYAALCRAIATAPAIDYAAISAPFLLIAGAEDKSASLEGCQHIFDRVSSGHKKMEVLPQIGHWHCVEAPEEVGGLIREFAAAL

SEQ ID NO: 77 - protein, 276 aa, Aspergillus kawachii
MPHLKLRDGAELFYKDWGNPRGPIVTFSHGWPLSSDNWEQQMMYLGEHGYRVIAHDRRGHGRSTQTWDGNNMDTFVDDLEELFAHLKVQDAVMVGHSHGGGEVTHYLGKHGTKRFKKAVLIGAVPPLMVKSSGNPEGTDKSVFDSFRQAMRQDRAQFFLDVPSGPFFGFNRPGAKKSEGQIRSWWQQGMNTSFKSAYDSIKDFSETDFSEDLKRINIPVLVLHGDDDQVVPIEAAGLKSAKLLPQGKLKIYKGGSHAIHNINVEEVNQDLLDFIKS

SEQ ID NO: 78 - protein, 510 aa, Aspergillus nidulans
MADRPRVILEKSRTVRRRYQRSNKRFQFTASQIARIEREEEREIQAQKLREKEKRRIQLKKKKAEKEAKAREERIRRGIPDPNAPAVPASQPLLFNFIKRTPSVQPESESPEEQTRADPESHIETDTATESENDDLSFEDEEFDNILIALDGADHPTPVEKADGSGAKNNGNDEDEFSDCSAFYDEDLFKATEIVDQVSVATVLSTDSFEDDTAILLEKFVPDFDPGSSFDQELILTDIQPSPNKMPFVSINNHQLHYADSHPNGAPANGLTFFFIHGLGSSQNYYFPLLPHLTPQHRCITADTYGSGRSTYTGQSVSIASIADDVIGVLDALNIPQAVVVGHSMGGLVVTLLGSEHADRVKGIVAIGPTHPSETLTSVMRKRSATAAEGGMESLANSIPYQATGSAASPLASSFIRELVLGQNPKGYAALCQAIANAPTIDYSAINIPFLLIAGDEDKSASLEGCQYIFDRVSSANKKMEVLPQVGHWHCIEAPDTVGRAIAAFVKTSF

SEQ ID NO: 79 - protein, 276 aa, Aspergillus niger
MPHLKLRDGAELFYKDWGNPRGPIITFSHGWPLSSDNWENQMMFLGDHGYRVIAHDRRGHGRSTQTWDGNNMDTFVDDLEELFAHLKVQDAVMVGHSHGGGEVTHYLGKHGTGRFKKAVLIGAVPPLMLKGSGNPEGTDKSVFDSFRQAMRQDRAQFFLDVPSGPFFGFNRPHVQKSEGQIRSWWQQGMNTSFKSAYDSIRDFSETDFTEDLKKINIPVLVLHGDDDQVVPIEAAGLKSAKLLPQGKLKIYKGGSHAIHNINVDEVNQDLLDFIKS

SEQ ID NO: 80 - protein, 276 aa, Aspergillus oryzae
MPYLKLRDGAELFYKDWGNPDGEIVTFSHGWPLSSDNWENQMFFLAERGYRVIAHDRRGHGRSTQTWNGNNMDTFVDDLKELFEHLDIKGAMMVGHSHGGGEVTHFLGKHGTSRVKKAVLVGAVPPLMLKSAANPEGTDQSVFDSFRQAMRKDRAQFFLDVPSGPFFNFNRDGVQKSEGQIRSWWQQGMNTSFKTAYDAIKDFSETDFTEDLKKIDIPVLVLHGDDDQVVPIEASGHKSVKLLRHGKLKVYPGGSHAIHNINIEEVNKDLLDFLQS

SEQ ID NO: 81 - protein, 276 aa, Aspergillus sydowii
MPYLKLRDGAELFYKDWGNPKGPIVTFSHGWPLSSDNWEAQMFHLASNGYRVIGHDRRGHGRSTQTWDGNNMETFVDDLEELFAHLNVQDAVMVGHSHGGGEVTRYLGKHSTKRVKKAVLVGAVPPLMLKTSANPDGLDKSVFDSFRQAMENDRAQFFIDVPSGPFFGFNRDEDKKSQGQIWNWWRQGMNTSFKTAYDCVKDFSETDFTDDLKKIDIPVLVLHGDDDQVVPIQTTGARAAKLLPQGKLKVYKGGSHGVHNINVGEVNQDLLEFIKS

SEQ ID NO: 82 - protein, 266 aa, Aspergillus terreus
MPYLKLKDGADLFYKDWGNPTGEIVTFSHGWPLSSDNWENQMVFLADQGYRVIAHDRRGHGRSTQTWAGNNMDTTWDIQGATMVGHSHGGGEVTHFLGTYGTRRVKKAVLLGAVPPLMVKTEANPEGTDKAVFDSFRAAMHADRAQFFLDVPSGPFFGFNRPGAKKSEGQIRSWWQQGMNSSFKTTYDCIKDFSETDFTEDLKKIDIPVLVVHGDDDQVVPIQAAGEKSAKLLPQGKLKVYKGGSHAIHNISIDEFNKDLIDFIRS

SEQ ID NO: 83 - protein, 276 aa, Aspergillus tubingensis
MPHLKLRDGAELFYKDWGNPRGPIVTFSHGWPLSSDNWEQQMMYLGEHGYRVIAHDRRGHGRSTQTWDGNNMDTFVDDLEELFAHLKVQDAVMVGHSHGGGEVTHYLGKHGTKRFKKAVLIGAVPPLMVKSSGNPEGTDKSVFDSFRQAMRQDRAQFFLDVPSGPFFGFNRPGAKKSEGQIRSWWQQGMNTSFKSAYDSIKDFSETDFSEDLKKINIPVLVLHGDDDQVVPIEAAGLKSTKLLPQGKLKIYKGGSHAIHNINVDEVNQDLLDFIKS

SEQ ID NO: 84 - protein, 276 aa, Aspergillus wentii
MPYLKLRDGAELYYKDWGNRGGAMVTFSHGWPLSSDNWENQMMFLADHGYRVIAHDRRGHGRSTQTWHGNNMDTFVDDLEELFAHLKIKDAVMVGHSHGGGEVTHYLGKHGTSRFKKAVLVGAVPPLMVKTSANTEGTDKSVFDSFREAMRKDRAQFFLDVPSGPFFGFNRPDAQKSEGQIRSWWQQGMNTSFKSAYDSIKDFSETDFTEDLKKIDIPVLVLHGDDDQVVPIEASGLKSAKLLPQGTLKVYKGGSHAIHNINVDQVNKDLVDFIRS

SEQ ID NO: 85 - protein, 275 aa, Fusarium oxysporum
MSFFSTTKRIPLQDDVHLHVEQSFPSRQSQDHLTIIFLHFWGGSSKTWSPVNHLIAPTFPTVRIDFRGWGISTGPADETKYSILQLAKDIELVITRLDIQEYIIVGHSMGAKVAQAIAGRKLVSGLTDLVVLCPAPPTPLMLPEEMRDQQISAYNNTQNAEFVVLNVLTAKSLPGDTVKRTVEDMLKGCSSAKAAWPRYAMNEDILELAKRISVPSVVIAGGKDQVEPGARVKEEVSGNIPFAKFLVLEEVGHLAPLEAPEDVAEIISDFVSSSI

SEQ ID NO: 86 - protein, 267 aa, Penicillium digitatum
MPFVQANSHRLYYADSHPAGPPTPTGLTFIFIHGLGSSQNYYYPVIPHLTNLHRCITIDTYGAARSPYTNDTVTIPGIAEDVIGVLDSLHVPKAVVVGHSMGGLVVTELGARHPDRVQGVVAIGPTHPSDTLVTVMNKRSETVLEAGIEPMANTIPFGAVGSRSTPLHKAFIRELILGQDPKGYAALCRAIAAAKPADYAAVQAPFLLIAGEEDKSASMEGCQHIFDHVLSKQKSLEVLKGVGHWHCIEAADEVGELIATFANKVKA

SEQ ID NO: 87 - protein, 276 aa, Penicillium rubens
MPYLKLRDGAELYYKDWGNPEGAIVTFSHGWPLSSDNWENQMIYLADHGYRVIAHDRRGHGRSAQTWDGNNMDTFVDDLEELFAHLKVKDAVMVGHSHGGGEVTHYLGKHGIRRFKKAVLVGAVPPLMVKTSVNTEGTDKSVFDSFREAMRKDRAQFFLDVPSGPFFGFNRPGAQKSEGQIRSWWQQGMNTSFKSAYDSIKDFSETDFTEDLKKIDIPVLVLHGDDDQVVPIEASGLKSAKLLPQGRLRIYKGGSHAIHNINIDEVNKDLLGFIKE

SEQ ID NO: 88 - protein, 274 aa, Trichoderma harzianum
MPFLKLQDRDRHLYWTLNAPPTTTSSSDDSITNIVLIHGLGSSSSYYAPVIPKLVDAGYTCLALDSHGAALTPLTGQGGSLDTIIDDIGAAVSALNISPSRTVIVGHSMGGIVAPEATLRYKFAGTVLLGPVYPNETLTKVLQDRIAKVQQNGMDPLVDVIPIVGTASSSTPLQRAFIRTLLLAQTPEGYIANCKAIASASIPDYASISTPLLLINGAEDFVCPIELSQKVFDSWGSKDKNLQILDKTGHWYCIESPEQVGDAIVKFVSGIKTA

SEQ ID NO: 89 - protein, 273 aa, Trichoderma virens
MPFLKLQDRDRQLYYTWSAPPTAASSSDKITTVILIHGLGSSSSYYAPVIPKLVDAGYTCLALDSHGAALTPLTGEGGSLDTIIDDIGAAVSALDISPSRTVIVGHSMGGIVAPEAALRYQFAGTVLLGPVYPNAALTKVLQDRITKVQQNGMDSLADIIPIVGTASSSTPLQRAFIRTLLLSQTPEGYIANCKAIASASVPDYASIGTPLLLINGAEDFVCPIELSQKVYDSWGSKDKKLQILDRTGHWYCIESPEHVGTAIVQFVNGIKTA

SEQ ID NO: 90 - protein, 270 aa, Clostridium sp.
MGYYIRVEPNVKIYVEDLNPEGNKTILFIHGWPGSHKLFEYQFDQLPKMGYRCIGIDTRGFGNSDKPWKGYDFNRLSDDVRCVIDTLKLHDIILAGHSNGGAIAVRYMARHKGYGISKLVLIAAVAPSLIKRPGFPYGLDKETVVQLINGTYTDRPKMLEGFGDLFFFQHITQPFSNWFFQLGLQAAGWSTAAIANTWINEVLFSDLETINVPTLIIHGIHDKIVPFELGEIQNKSIKNSKLIPFNYSGHGVFYDEKDKFNEELVKFAEE

SEQ ID NO: 91 - DNA, 813 bp, Artificial Sequence
  Clostridium sp. alfa-beta hydrolase DCM59_17820 nucleotide sequence codon optimised to Escherichia coli
atgggttactatatccgtgtggagccgaacgttaaaatttacgtggaggacctgaacccggaaggcaacaagaccatcctgttcattcacggttggccgggcagccacaaactgtttgaataccagttcgaccaactgccgaagatgggttatcgttgcatcggcattgatacccgtggtttcggcaacagcgacaagccgtggaaaggttatgattttaaccgtctgagcgacgatgtgcgttgcgttatcgacaccctgaaactgcacgatatcattctggcgggtcacagcaacggtggcgcgattgcggttcgttacatggcgcgtcacaaaggttatggcatcagcaagctggttctgattgcggcggtggcgccgagcctgatcaaacgtccgggtttcccgtacggcctggataaggagaccgtggttcagctgattaacggtacctataccgaccgtccgaagatgctggaaggtttcggcgacctgttcttcttccagcacatcacccaaccgtttagcaactggtttttccaactgggtctgcaagcggcgggttggagcaccgcggcgattgcgaacacctggattaacgaggttctgtttagcgacctggaaaccatcaacgtgccgaccctgatcattcacggtatccacgataaaattgtgccgttcgagctgggcgaaatccaaaacaagagcattaaaaacagcaagctgattccgtttaactacagcggtcacggcgtgttctacgacgagaaggataaattcaacgaggaactggtgaagtttgcggaggaataa

SEQ ID NO: 92 - protein, 270 aa, Sporomusa sphaeroides
MGLYIDVESDVNVYVEDLNPEGDKTILFIHGWPANYQMFEYQFNQLPKMGYRCVGVDTRGFGNSDKPWRGYDYDTLADDIRCVVEALDLHDFTLAGHSTGGAIAIRYMARHRGFGVSKLALFAAAAPSLIQRPYFPYGLQREDVTRIIQGTYTDRPEMLRGFGNMFFFQHITQPLSDWFFNLGLQAASWSTAAVAAAWLDEELFSDLGKIHVPTLILHGIHDKVCLFPLALAQKKGIRNAKLVPFQYSGHGLFYDECDKFNKELTDFIDE

SEQ ID NO: 93 - DNA, 813 bp, Artificial Sequence
  Sporomusa sphaeroides arylesterase SPSPH_10950 nucleotide sequence codon optimised to Escherichia coli
atgggtctgtacatcgacgttgaaagcgatgtgaacgtttatgtggaggacctgaacccggaaggtgataagaccatcctgttcattcacggctggccggcgaactaccaaatgtttgagtatcagttcaaccaactgccgaagatgggttaccgttgcgttggcgtggacacccgtggtttcggcaacagcgataaaccgtggcgtggttacgactatgataccctggcggacgatattcgttgcgtggttgaagcgctggacctgcacgattttaccctggcgggtcacagcaccggtggcgcgatcgcgattcgttacatggcgcgtcaccgtggtttcggcgttagcaaactggcgctgtttgcggcggcggcgccgagcctgatccagcgtccgtacttcccgtatggtctgcaacgtgaagacgtgacccgtatcattcagggcacctataccgatcgtccggagatgctgcgtggttttggcaacatgttctttttccagcacattacccaaccgctgagcgactggtttttcaacctgggtctgcaggcggcgagctggagcaccgcggcggttgcggcggcgtggctggacgaggaactgttcagcgatctgggcaagatccacgttccgaccctgatcctgcacggcattcacgataaagtgtgcctgttcccgctggcgctggcgcaaaagaaaggcattcgtaacgcgaagctggtgccgtttcagtacagcggtcacggcctgttttatgacgagtgcgataagttcaacaaagaactgaccgactttatcgatgagtaa

SEQ ID NO: 94 - protein, 278 aa, Microbacterium sp.
MSEITAHHGLLTDIDLHVDDTGGTGRPVVLIHGWPLSGESWAHQVPALEAAGHRVVTYDRRGFGRSDKPRTGYDYDTFADDLEAVLTALDLRDVTLVGFSMGGGEVARYLTRHGSGRVHSVVFAAAVPPYLAKTEDNPEGPLDDETADGMKQGLKGDEKAFYHQFTTDFFSVDGALAVPAAEQVEAERLANQADHHAALKSMEAFATTDFREDLPNVTVPTLVIHGDSDATVPFEGSGKRTHEAIAGSELHVVAGAPHGVNVSHADEFNRVLIDFLQR

SEQ ID NO: 95 - DNA, 837 bp, Artificial Sequence
  Microbacterium sp. bromoperoxidase DBR36_08250 nucleotide sequence codon optimised to Escherichia coli
atgagcgagatcaccgcgcaccacggcctgctgaccgacattgatctgcacgttgacgataccggtggcaccggtcgtccggtggttctgatccatggttggccgctgagcggcgagagctgggcgcaccaagtgccggcgctggaagcggcgggtcaccgtgtggttacctacgaccgtcgtggcttcggtcgtagcgataaaccgcgtaccggttacgactatgatacctttgcggacgatctggaggcggttctgaccgcgctggacctgcgtgatgtgaccctggttggcttcagcatgggtggcggtgaagtggcgcgttacctgacccgtcatggtagcggtcgtgttcatagcgtggtttttgcggcggcggtgccgccgtatctggcgaagaccgaggacaacccggagggtccgctggacgatgagaccgcggacggtatgaagcagggcctgaaaggtgatgaaaaggcgttctatcaccaatttaccaccgacttctttagcgtggatggtgcgctggcggtgccggcggcggaacaggtggaggcggaacgtctggcgaaccaagcggaccaccatgcggcgctgaaaagcatggaggcgttcgcgaccaccgactttcgtgaagatctgccgaacgtgaccgttccgaccctggttatccacggcgacagcgatgcgaccgtgccgtttgagggtagcggcaagcgtacccacgaggcgattgcgggtagcgaactgcatgtggttgcgggtgcgccgcacggtgtgaacgttagccacgcggacgaattcaaccgtgttctgattgattttctgcaacgttaa

SEQ ID NO: 96 - protein, 260 aa, Alkalihalobacillus okhensis
MGYFVEVEPDVNLYVEDVNPEGRKTLVFIHGWPLSHEQFEYQFNILPSMGYRCVGIDWRGFGKSDRPWHGYTYNRLADDIRTVIDTLELDGVTLVGHSTGGAIAIRYVTRHNGHGVSQLVLVDAAAPVGFTEDTANSLLEQTLNDRPKMMQGVTDTFFFQSISKPFSDWFNQLGVRAASWSTAAVIHLLRDETLYEDMHNIHVPTLIIHGVHDKVIPFEQALELHQNIRNSQLVPFEFSGHGPFWEEREKFNQLLMEFSS

SEQ ID NO: 97 - DNA, 783 bp, Artificial Sequence
  Alkalihalobacillus okhensis esterase LQ50_02985 nucleotide sequence codon optimised to Escherichia coli
atgggttacttcgttgaggtggaaccggacgtgaacctgtatgtggaggatgttaacccggaaggtcgtaagaccctggtttttatccacggctggccgctgagccacgagcagtttgaataccaattcaacatcctgccgagcatgggttatcgttgcgtgggcattgactggcgtggtttcggcaaaagcgatcgtccgtggcacggttacacctataaccgtctggcggacgatatccgtaccgttattgacaccctggagctggatggtgttaccctggttggccacagcaccggtggcgcgatcgcgattcgttacgtgacccgtcacaacggtcacggcgttagccaactggtgctggttgacgcggcggcgccggtgggttttaccgaggataccgcgaacagcctgctggaacagaccctgaacgaccgtccgaagatgatgcaaggtgttaccgataccttctttttccagagcattagcaaaccgtttagcgactggttcaaccagctgggcgtgcgtgcggcgagctggagcaccgcggcggttatccacctgctgcgtgacgagaccctgtatgaagatatgcacaacattcacgtgccgaccctgatcattcacggcgtgcacgataaggttatcccgttcgagcaggcgctggaactgcaccaaaacattcgtaacagccagctggttccgtttgaattcagcggtcacggcccgttttgggaggaacgtgagaaattcaaccagctgctgatggaatttagcagctaa

SEQ ID NO: 98 - protein, 269 aa, Paenibacillus sp.
MGRYVQVEPNVKVYVEDIGEGTPVVFLHGWPVNYKMFEYQLNVLPNHGIRAIAIDFRGYGLSDKPSTGYDYDRMADDVRAVIDELELKDAVLAGFSMGGAIAVHYMARHKGHGVSKLALLSAAAPVFTQREGYPYGLTPEQLNEQIIEPIFADRPKLLETFGGMFFAKKHSQPFMDWFHALGMEASSYGTIRSAIALRDEDLRGELSSIQVPTAILHGKKDEICPFDFAEQMHKGIASSQLIAFEESGHGAFYDELEKFNSELIRFIQS

SEQ ID NO: 99 - DNA, 810 bp, Artificial Sequence
  Paenibacillus sp. hydrolase C161_02220 nucleotide sequence codon optimised to Escherichia coli
atgggtcgttacgtgcaggttgaaccgaacgtgaaggtttatgtggaggacatcggtgaaggcaccccggtggtttttctgcacggttggccggttaactacaaaatgttcgagtatcaactgaacgtgctgccgaaccacggcattcgtgcgatcgcgattgactttcgtggttacggcctgagcgataagccgagcaccggttacgactatgatcgtatggcggacgatgttcgtgcggtgatcgacgagctggaactgaaagatgcggttctggcgggctttagcatgggtggcgcgattgcggttcactatatggcgcgtcacaagggtcacggcgtgagcaaactggcgctgctgagcgcggcggcgccggtgttcacccagcgtgaaggttacccgtatggcctgaccccggagcagctgaacgaacaaatcattgagccgatctttgcggaccgtccgaagctgctggaaaccttcggtggcatgttctttgcgaagaaacacagccagccgttcatggattggtttcacgcgctgggtatggaggcgagcagctacggcaccatccgtagcgcgattgcgctgcgtgacgaagatctgcgtggtgagctgagcagcatccaagttccgaccgcgattctgcacggcaagaaagacgaaatctgcccgttcgattttgcggagcagatgcacaagggtatcgcgagcagccaactgattgcgttcgaggaaagcggtcacggcgcgttctatgatgagctggaaaaatttaacagcgagctgatccgtttcattcaaagctaa

SEQ ID NO: 100 - protein, 271 aa, Clostridium carnis
MGYYIKAESNVKIYVEDLNENGTETILFLHGWPGDHNLFEYQFDYFSKMGYRCIGLDQRGFGESDKPLNGYDYDTLSDDVRCVIDQLQLKNVTLLGHSTGGAIAIRYMARHDECGISKLVLCGAAAPSLIRRSYFHYGLKEEAVKEIINETYIDRPKMLANFSKMFFYKPISKPFSDWFLNLGLKAAGWSTAAIANTWLDEESLFLDMKKINAPTLIIHGIHDKVCLFPLAEQQKKLIKNSKLVKMQNSGHGSFYDEREKFNNEVKCFIES

SEQ ID NO: 101 - DNA, 816 bp, Artificial Sequence
  Clostridium carnis prolyl iminopeptidase NCTC10913_04642 nucleotide sequence codon optimised to Escherichia coli
atgggttactatatcaaagcggaaagcaacgtgaagatttacgttgaggacctgaacgaaaacggcaccgagaccatcctgtttctgcacggttggccgggcgaccacaacctgttcgaataccagttcgattattttagcaaaatgggttatcgttgcattggcctggaccaacgtggttttggcgagagcgataagccgctgaacggttacgactatgataccctgagcgacgatgtgcgttgcgttatcgaccagctgcaactgaaaaacgttaccctgctgggtcacagcaccggtggcgcgatcgcgattcgttacatggcgcgtcacgatgagtgcggtatcagcaagctggtgctgtgcggtgcggcggcgccgagcctgattcgtcgtagctactttcactatggtctgaaagaggaagcggttaaggaaatcattaacgagacctacatcgaccgtccgaaaatgctggcgaacttcagcaagatgttcttttataaaccgattagcaagccgttcagcgattggtttctgaacctgggtctgaaagcggcgggttggagcaccgcggcgattgcgaacacctggctggacgaggaaagcctgtttctggatatgaagaaaatcaacgcgccgaccctgatcattcacggcattcacgacaaggtgtgcctgttcccgctggcggaacagcaaaagaaactgatcaagaacagcaaactggttaagatgcagaacagcggtcacggcagcttctatgatgagcgtgaaaagttcaacaacgaggttaagtgcttcattgagagctaa

SEQ ID NO: 102 - protein, 202 aa, Bacillus licheniformis
MAYELPELPYAYDALEPHIDKETMNIHHTKHHNTYVTKLNEAVAGKQDLESKSVEELVANLDAVPENIRTAVRNNGGGHANHSLFWKLLSPNGGGAPTGELAEAINSKFGSFDQFKEDFAAAAAGRFGSGWAWLVVNNGELEITSTPNQDSPLSEGKTPILGLDVWEHAYYLNYQNRRPDYIKAFWNVVNWDEVARLYSEAK

SEQ ID NO: 103 - DNA, 609 bp, Bacillus licheniformis
atggcttacgaacttccagaattaccttatgcgtacgatgctttagaaccgcatatcgacaaggaaactatgactattcaccatacgaaacaccacaacacatacgtgacaaatttaaacaaagcggttgaaggaaacactgctcttgccaacaaatcagttgaagagcttgttgcagatcttgattctgttcctgaaaacatccgcactgcagtccgcaacaacggcggcggacacgcgaaccacaaattattctggacacttttatctccaaacggcggaggcgaaccgactggcgcgcttgctgaagagatcaacagtgtatttggaagctttgacaaattcaaagagcaattcgcagcagcagcagctggacgcttcggttctggctgggcatggcttgttgtgaacaacggtaaacttgaaatcacaagcacgccaaaccaagattctccgctttcagaaggcaaaacgcctatcttaggtcttgacgtttgggagcatgcttactaccttaactatcaaaaccgccgtcctgattacatttcagctttctggaatgttgtaaactgggacgaagtggctcgtctatacagcgaagcaaaataa

SEQ ID NO: 104 - DNA, 73 bp, Artificial Sequence
  oPlastBug-242 oligonucleotide
tagaaataattttgtttaactttaagaaggagatatatccatggcttacaaacttccagaattaccttatgct

SEQ ID NO: 105 - DNA, 67 bp, Artificial Sequence
  oPlastBug-243 oligonucleotide
caagctgggatttaggtgacactatagaatactcaagcttttattttgcttcgctgtaaaggcgtgc

(19) United States
(12) Patent Application Publication
(10) Pub. No.: US 2024/0218146 A1
(43) Pub. Date: Jul. 4, 2024

(54) ENZYMES, MICRO-ORGANISMS AND USES THEREOF, AND A METHOD OF DEGRADING A POLYOLEFIN

(71) Applicant: TEKNOLOGIAN TUTKIMUSKESKUS VTT OY, Espoo (FI)

(72) Inventors: Kari KOIVURANTA, Espoo (FI); Irina TSITKO, Espoo (FI); Sandra CASTILLO, Espoo (FI); Heli NYGREN, Espoo (FI)

(21) Appl. No.: 18/286,324

(22) PCT Filed: Apr. 12, 2022
(86) PCT No.: PCT/FI2022/050238

§ 371 Date: Oct. 10, 2023

(30) Foreign Application Priority Data
Apr. 13, 2021 (FI) 20215439

(51) Int. Cl. C08J 11/10; C12N 9/02; C12N 9/08; C12N 9/18; C12N 9/48; C12N 9/64; C12N 9/88; C12P 5/00; C12P 5/02; C12P 7/26; C12P 7/6409

(52) U.S. Cl. CPC C08J 11/105; C12N 9/0065; C12N 9/0069; C12N 9/18; C12N 9/485; C12N 9/6408; C12N 9/88; C12P 5/002; C12P 5/026; C12P 7/26; C12P 7/6409; C12Y 111/01007; C12Y 111/0101; C12Y 111/01018; C12Y 111/02001; C12Y 113/11; C12Y 301/01001; C12Y 301/01002; C12Y 301/01003; C12Y 301/01024; C12Y 303/02003; C12Y 304/11005; C12Y 308/01005; C12Y 401/02

(57) ABSTRACT

The present invention relates to the fields of life sciences, micro-organisms and degradation of polyolefin polymers. Specifically, the invention relates to an isolated specific enzyme or a fragment thereof, wherein said enzyme or fragment is capable of degrading a polyolefin, and to a micro-organism or a host cell comprising the enzyme or a fragment thereof. Also, the present invention relates to a polynucleotide encoding the enzyme or fragment thereof, and to an expression vector or plasmid comprising the polynucleotide of the present invention. And still, the present invention relates to use of the enzyme, fragment, micro-organism, host cell, polynucleotide, expression vector or plasmid of the present invention for degrading a polyolefin: to a method of degrading a polyolefin with the specific enzyme or a fragment thereof: and to a method of producing the enzyme or fragment thereof of the present invention.

### FIELD OF THE INVENTION

The present invention relates to the fields of life sciences, micro-organisms and degradation of polyolefin polymers. Specifically, the invention relates to an isolated specific enzyme or a fragment thereof, wherein said enzyme or fragment is capable of degrading a polyolefin, and to a micro-organism or a host cell comprising the enzyme or a fragment thereof. Also, the present invention relates to a polynucleotide encoding the enzyme or fragment thereof, and to an expression vector or plasmid comprising the polynucleotide of the present invention. And still, the present invention relates to use of the enzyme, fragment, micro-organism, host cell, polynucleotide, expression vector or plasmid of the present invention for degrading a polyolefin; to a method of degrading a polyolefin with the specific enzyme or a fragment thereof; and to a method of producing the enzyme or fragment thereof of the present invention.

### BACKGROUND OF THE INVENTION

With the existing plastic recycling systems (mechanical and chemical) not all plastic waste can be recycled. This is partly due to the quality of plastic wastes (mixed plastic, dirty plastics). Additionally, the existing recycling methods need much energy. Biotechnical recycling could be utilized for improving the range of recycling methods and for enabling cost effective and more efficient recycling of plastics.

Removal of highly stable and durable polyolefin polymers including but not limited to plastics comprising polyolefins from the environment by using microbes or microbial enzymes is of high interest. In general, biotechnical plastic degradation is not common yet. Only few micro-organisms or enzymes capable of degrading polyolefins have been discovered and said micro-organisms or enzymes are not effective. For example, Santo M. et al. (2013, International Biodeterioration & Biodegradation 84, 204-210) describe degradation of polyethylene (PE) with an extracellular fraction comprising several different enzymes obtained from a Rhodococcus ruber cell culture. However, for PE or other polyolefins, recycling systems utilizing specific enzymes including but not limited to isolated and/or purified enzymes, or micro-organisms comprising said specific enzymes are under development. Indeed, it is very difficult to degrade polyolefins with enzymes or micro-organisms.

Micro-organisms or enzymes are needed for rapid and simple degradation and recycling of polyolefins. There remains a significant unmet need for specific micro-organisms and enzymes for effective degradation of polyolefins.

### BRIEF DESCRIPTION OF THE INVENTION

By biotechnical degradation and tools of the present invention it is possible to degrade and therefore recycle hydrocarbon chains such as plastics or synthetic polymers and more specifically polyolefins. Furthermore, the tools of the present invention can be used e.g. for upcycling hydrocarbon chains or polyolefins i.e. for modifying a non-biodegradable plastic or polyolefin (e.g. PE) to a biodegradable plastic (such as polyhydroxyalkanoate (PHA)) or fatty acid derived products (such as PHA and/or diacids) by micro-organisms and enzymes.

The objects of the invention, namely methods and tools for degrading hydrocarbon chains or polyolefins are achieved by utilizing a specific enzyme or enzymes, or a specific micro-organism or micro-organisms (e.g. a bacterium/bacteria and/or fungus/fungi) comprising said enzyme(s).

The present invention provides methods and tools which enable biotechnical degradation of polyolefins. Said methods and tools provide surprising degradation effects on polyolefins or on a combination of specific plastics or polymers comprising polyolefins. Also, the present invention can overcome the problems of the prior art including but not limited to ineffective or slow biotechnical degradation of polyolefin polymers. Furthermore, the specific enzyme or micro-organism of the present invention enable degradation methods at low temperatures, e.g. at a temperature below 100° C., indicating low energy need and costs.

Also, the inventors of the present disclosure surprisingly found out that unique or specific degradation products can be obtained with the present invention.

Novel biotechnical plastic recycling systems can be generated based on the enzyme, micro-organism or method of the present invention.

Specifically, the present invention relates to a method of degrading a polyolefin, said method comprising
providing a material comprising a polyolefin and an enzyme or a fragment thereof capable of degrading the polyolefin, and
allowing said enzyme or fragment thereof to degrade the polyolefin, wherein the enzyme or fragment thereof comprises a catalytic triad Ser-Asp-His corresponding to amino acids Ser97, Asp221 and His249 presented in SEQ ID NO: 2.

Also, the present invention relates to a method of degrading a polyolefin, said method comprising
providing a material comprising a polyolefin and an enzyme or a fragment thereof capable of degrading the polyolefin, and
allowing said enzyme or fragment thereof to degrade the polyolefin, wherein the enzyme or a fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, or 8.

Also, the present invention relates to an isolated enzyme or a fragment thereof comprising a catalytic triad Ser-Asp-His corresponding to amino acids Ser97, Asp221 and His249 presented in SEQ ID NO: 2, wherein said enzyme or fragment is capable of degrading a polyolefin.

Also, the present invention relates to an isolated enzyme or a fragment thereof having at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, or 8, wherein said enzyme or fragment is capable of degrading a polyolefin.

Furthermore, the present invention relates to a micro-organism or a host cell comprising an enzyme or a fragment thereof comprising a catalytic triad Ser-Asp-His corresponding to amino acids Ser97, Asp221 and His249 presented in SEQ ID NO: 2, wherein said enzyme or fragment is capable of degrading a polyolefin.

Furthermore, the present invention relates to a micro-organism or a host cell comprising an enzyme or a fragment thereof having at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, or 8, wherein said enzyme or fragment is capable of degrading a polyolefin.

Still, the present invention relates to a polynucleotide encoding the enzyme or fragment thereof of the present invention.

Still, the present invention relates to an expression vector or plasmid comprising the polynucleotide of the present invention.

And still, the present invention relates to use of the enzyme, fragment, micro-organism, host cell, polynucleotide, expression vector or plasmid of the present invention or any combination thereof for degrading a polyolefin.

Still furthermore, the present invention relates to a method of producing the enzyme or fragment thereof of the present invention, wherein a recombinant micro-organism or host cell comprising the polynucleotide encoding the enzyme or fragment thereof of the present invention is allowed to express said enzyme or fragment thereof.

Other objects, details and advantages of the present invention will become apparent from the following drawings, detailed description and examples.

The objects of the invention are achieved by compounds, uses and methods characterized by what is stated in the independent claims. The preferred embodiments of the invention are disclosed in the dependent claims.

### BRIEF DESCRIPTION OF THE DRAWINGS

FIG. 1 shows results from the GC-MS run. With Bacillus licheniformis, Rhodococcus ruber and Bacillus cereus enzyme samples (cell extracts) several peaks appeared which were missing from control sample (in control an empty plasmid) with UV treated polyethylene powder.

FIG. 2 shows results from the GC-MS run. With Bacillus licheniformis, Rhodococcus ruber and Bacillus flexus enzyme samples (cell extracts) several peaks appeared which were missing from control sample (in control an empty plasmid) with polypropylene powder.

FIG. 3 shows results from the GC-MS run. With Rhodococcus ruber enzyme sample (supernatant sample) several peaks appeared which were lower or missing in control sample (in control an empty plasmid) with polyethylene powder.

FIG. 4 shows results from the GC-MS run. With Bacillus flexus and Bacillus licheniformis enzyme sample (supernatant samples without hydrogen peroxide) several peaks appeared which were lower or missing in control sample (in control an empty plasmid) with polyethylene powder.

FIG. 5 shows results from the GC-MS run. With Bacillus cereus enzyme sample (supernatant sample) several alkane like compounds appeared or was higher than in control sample (in controls an empty plasmid) with LDPE film.

FIG. 6 shows results from the GC-MS run. With Bacillus flexus enzyme sample (supernatant sample) several alkane like compounds appeared which were missing from control sample (in controls an empty plasmid) with HDPE film.

FIG. 7 shows a plasmid map of plasmid pPB072.

FIG. 8 shows results from the GC-MS run. With cell extract sample of Yarrowia lipolytica expressing Bacillus cereus chloroperoxidase enzyme several peaks appeared which were missing from control sample (in control wild type Yarrowia lipolytica) with polypropylene powder.

FIG. 9 shows results from the GC-MS run. With cell extract sample of Yarrowia lipolytica expressing Bacillus cereus chloroperoxidase enzyme several peaks appeared which were lower or missing from control sample (in control wild type Yarrowia lipolytica) with polyethylene powder.

FIG. 10 shows results from the GC-MS run. With supernatant sample of Yarrowia lipolytica expressing Bacillus cereus chloroperoxidase enzyme without hydrogen peroxide addition several peaks appeared which were lower or missing from control sample (in control wild type Yarrowia lipolytica) with polyethylene powder.

FIG. 11 shows an alignment of several consensus amino acids of micro-organisms based on Bacillus cereus chloroperoxidase amino acid positions His29, Gly30, Gln41, Arg51, Asp56, Arg58, Gly61, Ser63, Gly69, Asp78, Leu86, Ser97, Gly99, Pro135, Gly218, Asp221 and His249.

FIG. 12 shows a pairwise alignment of Streptomyces griseus (SEQ ID NO: 68) and B. cereus (SEQ ID NO: 2) chloroperoxidases. Detected consensus amino acids have been marked with bold.

FIG. 13 shows an alignment of several consensus amino acids of filamentous fungi based on Bacillus cereus chloroperoxidase amino acid positions His29, Asp56, Gly59, Val94, Gly95, Ser97, Gly99, Pro126, Pro212, Gly218, Asp221, His249 and Phe266. Amino acid sequence of Aspergillus nidulans polypeptide is partial.

FIG. 14 shows a pairwise alignment of Aspergillus niger (SEQ ID NO: 79) and Bacillus cereus (SEQ ID NO: 2) chloroperoxidases. Detected consensus amino acids have been marked with bold.

FIG. 15 shows two-dimensional structure (alfa helixes and beta sheets) of Bacillus cereus chloroperoxidase (SEQ ID NO: 2) and localisation of the important amino acids for enzyme activity. Alfa helixes are underlined and numbered with Arabic numbers. Beta sheets are in Italics and numbered with Roman numbers. Important amino acids (Ser97, Gly99, Pro126, Gly218, Asp221 and His249) are in bold.

FIG. 16 shows results from the GC-MS run. With Clostridium sp, Sporomusa sphaeroides, Microbacterium sp., Alkalihalobacillus okhensis, Paenibacillus sp. and Clostridium carnis enzyme samples (supernatant samples) peaks appeared which were lower or missing in control sample (in control an empty plasmid) with polyethylene powder.

FIG. 17 shows results from the GC-MS run. With Sporomusa sphaeroides enzyme sample (supernatant sample) peaks appeared which were lower or missing in control sample (in control an empty plasmid) with polypropylene powder.

FIG. 18 shows results from the GC-MS run. With Paenibacillus sp. hydrolase and Bacillus licheniformis superoxide dismutase enzyme samples (supernatant samples) peaks appeared which were higher than in the enzyme samples having Paenibacillus sp. or Bacillus licheniformis superoxide dismutase enzyme samples alone. These peaks were lower or missing in control sample (in control an empty plasmid) with polyethylene powder.

### SEQUENCE LISTING

SEQ ID NO: 1: Bacillus cereus chloroperoxidase nucleotide sequence;

SEQ ID NO: 2: Bacillus cereus chloroperoxidase amino acid sequence;

SEQ ID NO: 3: Bacillus flexus chloroperoxidase nucleotide sequence;

SEQ ID NO: 4: Bacillus flexus chloroperoxidase amino acid sequence;

SEQ ID NO: 5: Bacillus licheniformis chloroperoxidase nucleotide sequence;

SEQ ID NO: 6: Bacillus licheniformis chloroperoxidase amino acid sequence;

SEQ ID NO: 7: Rhodococcus ruber chloroperoxidase nucleotide sequence;

SEQ ID NO: 8: Rhodococcus ruber chloroperoxidase amino acid sequence;

SEQ ID NO: 9: oPlastBug-120 oligonucleotide;

SEQ ID NO: 10: oPlastBug-121 oligonucleotide;

SEQ ID NO: 11: oPlastBug-232 oligonucleotide;

SEQ ID NO: 12: oPlastBug-233 oligonucleotide;

SEQ ID NO: 13: oPlastBug-234 oligonucleotide;

SEQ ID NO: 14: oPlastBug-235 oligonucleotide;

SEQ ID NO: 15: oPlastBug-236 oligonucleotide;

SEQ ID NO: 16: oPlastBug-237 oligonucleotide;

SEQ ID NO: 17: Bacillus cereus chloroperoxidase nucleotide sequence codon optimised to Yarrowia lipolytica;

SEQ ID NO: 18: Achromobacter xylosoxidans chloroperoxidase amino acid sequence;

SEQ ID NO: 19: Acinetobacter baumannii chloroperoxidase amino acid sequence;

SEQ ID NO: 20: Acinetobacter pittii chloroperoxidase amino acid sequence;

SEQ ID NO: 21: Arthrobacter sp. chloroperoxidase amino acid sequence;

SEQ ID NO: 22: Acidovorax sp. chloroperoxidase amino acid sequence;

SEQ ID NO: 23: Bacillus aryabhattai chloroperoxidase amino acid sequence;

SEQ ID NO: 24: Bacillus mycoides chloroperoxidase amino acid sequence;

SEQ ID NO: 25: Bacillus sp. chloroperoxidase amino acid sequence;

SEQ ID NO: 26: Bacillus subtilis chloroperoxidase amino acid sequence;

SEQ ID NO: 27: Bacillus thuringiensis chloroperoxidase amino acid sequence;

SEQ ID NO: 28: Bacillus vallismortis chloroperoxidase amino acid sequence;

SEQ ID NO: 29: Brevibacillus borstelensis chloroperoxidase amino acid sequence;

SEQ ID NO: 30: Brevibacillus brevis chloroperoxidase amino acid sequence;

SEQ ID NO: 31: Brevibacillus sp. chloroperoxidase amino acid sequence;

SEQ ID NO: 32: Citrobacter amalonaticus chloroperoxidase amino acid sequence;

SEQ ID NO: 33: Comamonas sp. chloroperoxidase amino acid sequence;

SEQ ID NO: 34: Cupriavidus necator chloroperoxidase amino acid sequence;

SEQ ID NO: 35: Enterobacter asburiae chloroperoxidase amino acid sequence;

SEQ ID NO: 36: Enterobacter sp. chloroperoxidase amino acid sequence;

SEQ ID NO: 37: Flavobacterium sp. chloroperoxidase amino acid sequence;

SEQ ID NO: 38: Klebsiella pneumoniae chloroperoxidase amino acid sequence;

SEQ ID NO: 39: Leucobacter sp. chloroperoxidase amino acid sequence;

SEQ ID NO: 40: Lysinibacillus xylanilyticus chloroperoxidase amino acid sequence;

SEQ ID NO: 41: Meyerozyma guilliermondii chloroperoxidase amino acid sequence;

SEQ ID NO: 42: Microbacterium paraoxydans chloroperoxidase amino acid sequence;

SEQ ID NO: 43: Micrococcus luteus chloroperoxidase amino acid sequence;

SEQ ID NO: 44 Micrococcus sp. chloroperoxidase amino acid sequence;

SEQ ID NO: 45: Ochrobactrum intermedium chloroperoxidase amino acid sequence;

SEQ ID NO: 46: Paenibacillus sp. chloroperoxidase amino acid sequence;

SEQ ID NO: 47: Pantoea sp. chloroperoxidase amino acid sequence;

SEQ ID NO: 48: Pseudomonas aeruginosa chloroperoxidase amino acid sequence;

SEQ ID NO: 49: Pseudomonas azotoformans chloroperoxidase amino acid sequence;

SEQ ID NO: 50: Pseudomonas chlororaphis chloroperoxidase amino acid sequence;

SEQ ID NO: 51: Pseudomonas fluorencens chloroperoxidase amino acid sequence;

SEQ ID NO: 52: Pseudomonas monteilii chloroperoxidase amino acid sequence;

SEQ ID NO: 53: Pseudomonas protegens chloroperoxidase amino acid sequence;

SEQ ID NO: 54: Pseudomonas putida chloroperoxidase amino acid sequence;

SEQ ID NO: 55: Pseudomonas sp. chloroperoxidase amino acid sequence;

SEQ ID NO: 56: Pseudomonas stutzeri chloroperoxidase amino acid sequence;

SEQ ID NO: 57: Pseudomonas syringae chloroperoxidase amino acid sequence;

SEQ ID NO: 58: Rahnella aquatilis chloroperoxidase amino acid sequence;

SEQ ID NO: 59: Ralstonia sp. chloroperoxidase amino acid sequence;

SEQ ID NO: 60: Rhodococcus rhodochrous chloroperoxidase amino acid sequence;

SEQ ID NO: 61: Rhodococcus ruber chloroperoxidase amino acid sequence;

SEQ ID NO: 62: Rhodococcus sp. chloroperoxidase amino acid sequence;

SEQ ID NO: 63: Serratia marcescens chloroperoxidase amino acid sequence;

SEQ ID NO: 64: Staphylococcus sp. chloroperoxidase amino acid sequence;

SEQ ID NO: 65: Stenotrophomonas maltophilia chloroperoxidase amino acid sequence;

SEQ ID NO: 66: Stenotrophomonas panacihumi chloroperoxidase amino acid sequence;

SEQ ID NO: 67: Stenotrophomonas sp. chloroperoxidase amino acid sequence;

SEQ ID NO: 68: Streptomyces griseus chloroperoxidase amino acid sequence;

SEQ ID NO: 69: Streptomyces sp. chloroperoxidase amino acid sequence;

SEQ ID NO: 70: Bacillus circulans chloroperoxidase amino acid sequence;

SEQ ID NO: 71: Aspergillus acidus chloroperoxidase amino acid sequence;

SEQ ID NO: 72: Aspergillus brasiliensis chloroperoxidase amino acid sequence;

SEQ ID NO: 73: Aspergillus clavatus chloroperoxidase amino acid sequence;

SEQ ID NO: 74: Aspergillus flavus chloroperoxidase amino acid sequence;

SEQ ID NO: 75: Aspergillus fumigatus chloroperoxidase amino acid sequence;

SEQ ID NO: 76: Aspergillus japonicus chloroperoxidase amino acid sequence;

SEQ ID NO: 77: Aspergillus kawachii chloroperoxidase amino acid sequence;

SEQ ID NO: 78: Aspergillus nidulans chloroperoxidase amino acid sequence;

SEQ ID NO: 79: Aspergillus niger chloroperoxidase amino acid sequence;

SEQ ID NO: 80: Aspergillus oryzae chloroperoxidase amino acid sequence;

SEQ ID NO: 81: Aspergillus sydowii chloroperoxidase amino acid sequence;

SEQ ID NO: 82: Aspergillus terreus chloroperoxidase amino acid sequence;

SEQ ID NO: 83: Aspergillus tubingensis chloroperoxidase amino acid sequence;

SEQ ID NO: 84: Aspergillus wentii chloroperoxidase amino acid sequence;

SEQ ID NO: 85: Fusarium oxysporum chloroperoxidase amino acid sequence;

SEQ ID NO: 86: Penicillium digitatum chloroperoxidase amino acid sequence;

SEQ ID NO: 87: Penicillium rubens chloroperoxidase amino acid sequence;

SEQ ID NO: 88: Trichoderma harzianum chloroperoxidase amino acid sequence;

SEQ ID NO: 89: Trichoderma virens chloroperoxidase amino acid sequence;

SEQ ID NO: 90: Clostridium sp. alfa-beta hydrolase DCM59_17820 amino acid sequence;

SEQ ID NO: 91: Clostridium sp. alfa-beta hydrolase DCM59_17820 nucleotide sequence codon optimised to Escherichia coli;

SEQ ID NO: 92: Sporomusa sphaeroides arylesterase SPSPH_10950 amino acid sequence;

SEQ ID NO: 93: Sporomusa sphaeroides arylesterase SPSPH_10950 nucleotide sequence codon optimised to Escherichia coli;

SEQ ID NO: 94: Microbacterium sp. bromoperoxidase DBR36_08250 amino acid sequence;

SEQ ID NO: 95: Microbacterium sp. bromoperoxidase DBR36_08250 nucleotide sequence codon optimised to Escherichia coli;

SEQ ID NO: 96: Alkalihalobacillus okhensis esterase LQ50_02985 amino acid sequence;

SEQ ID NO: 97: Alkalihalobacillus okhensis esterase LQ50_02985 nucleotide sequence codon optimised to Escherichia coli;

SEQ ID NO: 98: Paenibacillus sp. hydrolase C161_02220 amino acid sequence;

SEQ ID NO: 99: Paenibacillus sp. hydrolase C161_02220 nucleotide sequence codon optimised to Escherichia coli;

SEQ ID NO: 100: Clostridium carnis prolyl iminopeptidase NCTC10913_04642 amino acid sequence;

SEQ ID NO: 101: Clostridium carnis prolyl iminopeptidase NCTC10913_04642 nucleotide sequence codon optimised to Escherichia coli;

SEQ ID NO: 102: Bacillus licheniformis superoxide dismutase amino acid sequence;

SEQ ID NO: 103: Bacillus licheniformis superoxide dismutase nucleotide sequence;

SEQ ID NO: 104: oPlastBug-242 oligonucleotide;

SEQ ID NO: 105: oPlastBug-243 oligonucleotide.

### DETAILED DESCRIPTION OF THE INVENTION

The present invention concerns a method of degrading a polyolefin, wherein a specific enzyme or micro-organism of the present invention is used for degrading said polyolefin. In one embodiment of the present invention a polyolefin or a material comprising one or more polyolefins or types of polyolefin is allowed to contact with an enzyme or micro-organism capable of degrading the polyolefin(s). A material comprising one or more polyolefins or types of polyolefins can be any material including but not limited to plastics or polymers of fossil origin, bio-based polymers or plastic material, polymer composites, copolymers, packaging material, textile, waste material comprising plastics or synthetic polymers (e.g. oil-based and/or biobased), and multilayer materials or mixtures of materials comprising synthetic polymers or plastics and furthermore one or more materials such as paper and/or cardboard. In one embodiment of the invention the material comprising one or more polyolefins or types of polyolefins is a recycled material or from a recycled material.

As used herein, “a plastic” refers to a material comprising or consisting of synthetic and/or semi-synthetic organic compounds and having the capability of being molded or shaped. As used herein “a synthetic polymer” refers to a human-made polymer. Synthetic polymers can be classified into four main categories: thermo-plastics, thermosets, elastomers, and synthetic fibers.

As used herein polyolefin refers to a type of polymer produced from a simple olefin (e.g. called an alkene with the general formula CₙH₂ₙ) as a monomer. For example, polyethylene and polypropylene are common polyolefins. Depending on a polymerization method utilized for producing a polyolefin, the polyolefin hydrocarbon chain can sometimes comprise a specific group or groups such as a ketone group e.g. at the end of the chain. Polyolefins can be non-toxic, non-contaminating and lighter than water. In one embodiment the polyolefin is polyethylene (PE), cross-linked polyethylene (PEX or XLPE), ultra-high molecular weight polyethylene (UHMWPE), high-density polyethylene (HDPE), medium-density polyethylene (MDPE), linear low-density polyethylene (LLDPE), low density polyethylene (LDPE), very low-density polyethylene (VLDPE), polypropylene (PP), polymethylpentene (PMP), polybutene-1 (PB-1), polyisobutylene (PIB), or any combination thereof. In one embodiment the polyolefin is a polyethylene, polypropylene or a combination thereof.

Polyethylene (PE) (formula (C₂H₄)ₙ) consists of long chain polymers of ethylene and it can be produced as high-density (HDPE), medium-density (MDPE), or low-density polyethylene (LDPE). PE can be chemically synthesized by polymerization of ethane and it is highly variable since side chains can be obtained depending on the manufacturing process. LDPE has more branching than HDPE (i.e. has a high degree of short- and long-chain branching), and therefore it's intermolecular forces are weaker, its tensile strength is lower, and its resilience is higher. Also, because its molecules are less tightly packed and less crystalline due to the side branches, its density is lower. In one embodiment LDPE is defined by a density range of about 910-930 kg/m³, MDPE is defined by a density range of about 926 to 0.940 kg/m³, and/or the density range of HDPE is about 930 to 970 kg/m³.

Cross-linked polyethylene (PEX or XLPE) is a form of polyethylene with cross-linked bonds in the polymer structure, changing the thermoplastic to a thermoset. Indeed, crosslinking enhances the temperature properties of the base polymer and furthermore e.g. tensile strength, scratch resistance, and resistance to brittle fracture.

Ultra-high molecular weight polyethylene (UHMWPE) is a thermoplastic, and it is made up of extremely long chains of PE, which all align in the same direction. The extremely long chain can usually have a molecular mass between 3.5 and 7.5 mil-lion amu.

Linear low-density polyethylene (LLDPE) is a substantially linear PE with significant numbers of short branches. LLDPE differs structurally from conventional LDPE because of the absence of long chain branching.

Very low density polyethylene (VLDPE) is a type of LLDPE with higher levels of short-chain branches than standard LLDPE. VLDPE can be defined e.g. by a density range of 0.880-0.910 g/cm³.

Polypropylene (PP) (formula (C₃H₆)ₙ) is a thermoplastic, which can be produced e.g. via chain-growth polymerization from the monomer propylene. PP is partially crystalline and non-polar. Its properties are very similar to PE, but it is e.g. slightly harder and more heat resistant.

Polymethylpentene (PMP) (i.e. poly(4-methyl-1-pentene), formula (C₆H₁₂)ₙ) is a thermoplastic polymer of 4-methyl-1-pentene. It is a high-molecular weight hydrocarbon and an extremely low density olefinic commodity thermoplastic. PMP's chemical resistance is close to that of PP. Compared to PP it is more easily softened by unsaturated and aromatic hydrocarbons, and chlorinated solvents, and slightly more susceptible to attack by oxidizing agents.

Polybutene-1 (PB-1) (formula (C₄H₈)ₙ) is a high molecular weight, linear, isotactic, and semi-crystalline polymer. Polybutylene can be produced by polymerization of 1-butene using supported Ziegler-Netta catalysts.

Polyisobutylene (PIB) (formula (C₄H₈)ₙ) can be prepared by polymerization of isobutene. The molecular weight of the PIB can determine the application. For example, low MW PIBs can be used as plasticizers, and medium and high MW PIBs in adhesives.

In one embodiment of the invention the enzyme capable of degrading a polyolefin or a polyolefin containing material is from a bacterium (gram-positive or gram-negative) or fungus, and/or the micro-organism capable of degrading a polyolefin or a polyolefin containing material is a bacterium (gram-positive or gram-negative) or fungus. As used herein “fungus”, “fungi” and “fungal” refer to yeast and filamentous fungi (i.e. moulds). In one embodiment of the invention the fungus is a yeast or filamentous fungus.

As used herein, “degradation” of a polyolefin, plastic, synthetic or non-synthetic polymer refers to either partial or complete degradation of a polyolefin, plastic, synthetic or non-synthetic polymer to a shorter hydrocarbon chain, oligomers and/or monomers. Said degradation can also include lowering of the molecular weight of a polyolefin, hydrocarbon chain or polymer, lowering of the average molecular weight, lowering of the molar mass in the peak of maximum and/or increase in polydispersity of a polyolefin, hydrocarbon chain or polymer. Indeed, any loss in the chain length of a polyolefin, hydrocarbon chain or polymer can e.g. lower tensile strength. “Enzymatic or microbial degradation” refers to a degradation caused by an enzyme or micro-organism, respectively. According to some hypothesis, in the microbial degradation the larger polymers are initially degraded by secreted exoenzymes or by outer membrane bound enzymes into smaller subunits (different length oligomers) that can be incorporated into the cells of micro-organisms and further degraded through the classical degradation pathways to yield energy and/or suit as building blocks for catabolism or metabolism.

Many plastics or other materials are mixtures comprising synthetic or semi-synthetic polymers and furthermore solubilizers and optionally other chemical agents for altering the mechanical and physical properties of said plastics or materials. The solubilizers and other chemical compounds may also be targets of enzymatic or microbial biodegradation.

In one embodiment of the invention the enzyme (or a fragment thereof), micro-organism or host cell comprises PE, PEX, UHMWPE, HDPE, MDPE, LLDPE, LDPE, VLDPE, PP, PMP, PB-1, or PIB degrading activity, or any combination thereof; or is capable of degrading a polyethylene and/or a polypropylene. In one embodiment the enzymes, fragments, micro-organisms or host cells of the present invention can be capable of utilizing any polyolefins, including but not limited to short, medium-sized and/or long hydrocarbon chain polyolefins.

Degradation of a polyolefin or a material comprising a polyolefin can result in at least one or more degradation products. In one embodiment of the invention, at least one or more degradation products selected from the group consisting of an alkane, alkene, alkyne, cycloalkane, alkadiene, dione, ketone, fatty acid, alcohol, aldehyde, epoxy, diacid, 2.9-decanedione, 2.11-dodecanedione, 2-decanone, 2-dodecanone, 2-tetradecanone, 2-hexadecanone, 2-heptadecanone, 2-dotriacontanone, alkane like compounds and oxygen containing hydrocarbons are obtained or obtainable by the degradation of the polyolefin. For example, PE can be degraded to an alkane, alkene, alkyne, cycloalkane, alkadiene, ketone (e.g. ketone C2-C32), fatty acid, alcohol, aldehyde, diacid, dione, 2.9-decanedione, 2.11-dodecanedione, 2-decanone, 2-dodecanone, 2-tetradecanone, 2-hexadecanone, 2-heptadecanone and/or 2-dotriacontanone. And for example, PP can be degraded to an alkane, alkene, alkyne, cycloalkane, alkadiene, ketone (e.g. ketone C2-C32), fatty acid, alcohol, aldehyde, and/or diacid.

In one embodiment of the invention only the enzyme(s) or micro-organism(s), or a combination thereof is(are) needed for a biotechnical or enzymatic degradation of a polyolefin or a combination of different types of polyolefins. In other words, no other degradation methods such as UV light, mechanical disruption or chemical degradation are needed in said embodiment. In other embodiments, biotechnical, enzymatic or microbial degradation can be combined with one or more other degradation methods (e.g. non-enzymatic degradation methods) including but not limited to UV light, gamma irradiation, microwave treatment, mechanical disruption and/or chemical degradation. In one embodiment of the invention the method of degrading a polyolefin is a biotechnical method, or the method comprises degradation of the polyolefin polymer by non-enzymatic methods or means. One or more non-enzymatic, non-microbial or non-biotechnical degradation methods or steps including pretreatments can be carried out sequentially (e.g. before or after) or simultaneously with the biotechnical, microbial or enzymatic degradation. For example, solvents can be used for separating polymer chains from each other before enzymatic degradation of a polyolefin. One or more (pre)treatments with solvents enable micro-organisms, enzymes or fragments thereof to access and degrade polyolefin in the inner parts of the plastic material to be degraded. Suitable solvents for plastics or polyolefins include but are not limited to toluene, xylene, benzene, trichlorobenzene, trichloroethylene, and/or tetralin.

In one embodiment, the method of degrading a polyolefin comprises obtaining, recovering, removing, recycling and/or re-utilizing at least one of the degradation products.

In one embodiment the present invention concerns an isolated enzyme or a fragment thereof comprising a catalytic triad (such as Ser-Asp-His) e.g. corresponding to amino acids at positions 97, 221 and 249 presented for example in SEQ ID NO: 2, wherein said enzyme or fragment is capable of degrading a polyolefin. Also, in one embodiment the present invention concerns a micro-organism or a host cell comprising an enzyme or a fragment thereof comprising a catalytic triad (such as Ser-Asp-His) e.g. corresponding to amino acids 97, 221 and 249 presented for example in SEQ ID NO: 2, wherein said enzyme or fragment is capable of degrading a polyolefin. In one embodiment of the invention the catalytic triad is Ser-Asp-His. In one embodiment the catalytic triad (such as Ser-Asp-His) is in amino acids corresponding to the amino acids 97 (such as Ser97), 221 (such as Asp221) and 249 (such as His249) corresponding to amino acids as presented in SEQ ID NO: 2.

In one embodiment, in addition to the catalytic triad amino acids Ser97, Asp221 and His249 corresponding to amino acids as presented in SEQ ID NO: 2, amino acids GLy99, Pro126 and Gly218 corresponding to amino acids as presented in SEQ ID NO: 2 were identified to have effect on the enzyme activity.

In one embodiment the present invention concerns an isolated enzyme or a fragment thereof comprising a catalytic triad (such as Ser-Asp-His) e.g., corresponding to amino acids at positions 97, 221 and 249 presented for example in SEQ ID NO: 2, and one or more amino acids selected from the group comprising or consisting of Gly99, Pro126 and Gly218 corresponding to the amino acid positions presented in SEQ ID NO:2, wherein said enzyme or fragment is capable of degrading a polyolefin. Also, in one embodiment the present invention concerns a micro-organism or a host cell comprising an enzyme or a fragment thereof comprising a catalytic triad (such as Ser-Asp-His) e.g., corresponding to amino acids 97, 221 and 249 presented for example in SEQ ID NO: 2, and one or more amino acids selected from the group comprising or consisting of Gly99, Pro126 and Gly218 corresponding to the amino acid positions presented in SEQ ID NO:2, wherein said enzyme or fragment is capable of degrading a polyolefin. In one embodiment of the invention the catalytic triad is Ser-Asp-His. In one embodiment the catalytic triad (such as Ser-Asp-His) is in amino acids corresponding to the amino acids 97 (such as Ser97), 221 (such as Asp221) and 249 (such as His249) corresponding to amino acids as presented in SEQ ID NO: 2.

Crystal structures of several enzymes e.g. microbial esterases have been described. According to structural studies, microbial esterases can have a canonical a/B-hydrolase fold comprising a central β-sheet surrounded by α-helices. The active site contains a catalytic triad formed by Ser-Asp-His residues, which is also found in other enzymes such as serine proteases (Lee C W et al. 2017, PLOS ONE 12 (1): e0169540; Nardini M, Dijkstra B W. 1999, Curr Opin Struct Biol. 9: 732-737).

Indeed, the catalytic residues of enzymes can constitute a highly conserved triad comprising a nucleophile (serine, cysteine or aspartic acid), an acidic residue and a histidine residue, optionally in any order or in the mentioned order nucleophile-acid-histidine. Depending on the enzyme in question the catalytic residues of the catalytic triad can be located far away from each other in the amino acid sequence of the enzyme. However, in the tertiary structure said catalytic residues can be very close to each other.

The nucleophile can be located e.g. in the ‘nucleophile elbow’, which can be easily approached by the substrate or the hydrolytic water molecule. In one embodiment the nucleophile is located after a β strand (such as (5). In one embodiment the nucleophile elbow comprises Sm—X-Nu-X—Sm (Sm=small residue, X=any residue, Nu=nucleophile). In one embodiment the nucleophile or a geometry of the nucleophile elbow stabilizes the negatively charged transition state that occurs during hydrolysis.

The acidic member of the catalytic triad can be located e.g. in a reverse turn often following a β strand (such as β6 or β7). In one embodiment the acidic residue al-lows the stabilization of the catalytic histidine during hydrolysis.

The histidine residue of the catalytic triad can be a single histidine residue or can be comprised of two or more histidine residues (e.g. after each other in the amino acid sequence). In some embodiments the histidine residue of the catalytic triad belongs to the histidine-containing loop, wherein optionally the shape and length of the loop can differ considerably among various enzymes. In one embodiment the histidine residue is located after a β strand (such as the last β strand of an enzyme).

In one embodiment the nucleophile (serine, cysteine or aspartic acid) is positioned after strand β5; an acidic residue (such as aspartic acid) is positioned after strand β6 or β7; and/or a conserved histidine residue is located after the last β strand of the enzyme.

As used herein “a catalytic triad” refers to three consensus or conserved amino acid residues of an enzyme, wherein said residues are in the active site of an enzyme and they all or at least some of them can act together with other residues to achieve nucleophilic catalysis. The triad residues can act together to make the nucleophile member of the triad highly reactive, optionally generating a covalent intermediate with a substrate that is then resolved to complete catalysis. It is also possible that the triad residues affect for example the reaction of hydrogen peroxide and a substrate without generating a covalent intermediate.

Catalysis of a substrate by catalytic triads can be performed for example in the following two stages. First, the activated nucleophile attacks the carbonyl carbon and forces the carbonyl oxygen to accept an electron, leading to a tetrahedral intermediate. The build-up of negative charge on this intermediate is typically stabilized by an oxyanion hole within the active site. The intermediate then collapses back to a carbonyl, ejecting the first half of the substrate, but leaving the second half still co-valently bound to the enzyme as an acyl-enzyme intermediate. The ejection of this first leaving group can be aided by donation of a proton by the base. The second stage of catalysis is the resolution of the acyl-enzyme intermediate by the attack of a second substrate. If this second substrate is a water molecule then the result is hydrolysis; if it is an organic molecule then the result is transfer of that molecule onto the first substrate. Attack by this second substrate forms a new tetrahedral intermediate, which resolves by ejecting the enzyme's nucleophile, releasing the second product and regenerating the free enzyme.

Alternatively or in addition to the catalysis of a substrate described in the above paragraph, one or more amino acid residues of a catalytic triad can affect the reaction of hydrogen peroxide with a substrate e.g. with an organic acid to form a peracid. For example, the greater separation of the active site serine and histidine residues of the catalytic triad might allow the binding of diatomic molecules, rather than water, and would allow a change of mechanism, whereby the histidine residue could act as a base.

In one embodiment an enzyme comprising a catalytic triad has different enzymatic activities at different pH, e.g., at high (such as 7.5-8.5, e.g. 8.0) and low pH (such as 5.0-6.0, e.g. 5.5). For example, pH can have an effect on whether the enzyme is an esterase or a peroxidase.

In one embodiment of the invention the enzyme or a fragment thereof comprises one or more relevant (e.g. consensus, conserved or catalytic) amino acids in specific positions selected from the group comprising or consisting of amino acid positions 29 (such as His29), 30 (such as Gly30), 41 (such as Gln41), 51 (such as Arg51), 56 (such as Asp56), 58 (such as Arg58), 61 (such as Gly61), 63 (such as Ser63), 69 (such as Gly69), 78 (such as Asp78), 86 (such as Leu86), 97 (such as Ser97), 99 (such as Gly99), 126 (such as Pro126), 135 (such as Pro135), 218 (such as Gly218), 221 (such as Asp221) and 249 (such as His249), and any combination thereof, e.g. as presented in SEQ ID NO: 2. In one embodiment the enzyme or a fragment thereof comprises one or more relevant amino acids in specific positions selected from the group comprising or consisting of amino acid positions 97 (e.g. Ser97), 221 (e.g. Asp221), 249 (e.g. His249), 29 (e.g. His29), 56 (e.g. Asp56), 99 (e.g. Gly99), 126 (e.g. Pro126) and 218 (e.g. Gly218), e.g. as presented in SEQ ID NO: 2. In a specific embodiment the enzyme or a fragment thereof comprises amino acids in specific positions selected from the group comprising or consisting of positions 97 (e.g. Ser97), 221 (e.g. Asp221) and 249 (e.g. His249), and optionally furthermore one or more of the following positions 29 (e.g. His29), 56 (e.g. Asp56), 99 (e.g. Gly99), 126 (e.g. Pro126) and 218 (e.g. Gly218) (such as at least 29 and 56; at least 29 and 99; at least 29 and 126; at least 29 and 218; at least 56 and 99; at least 56 and 218; at least 56 and 126; at least 99 and 218; at least 126 and 218; at least 29, 56 and 99; at least 29, 56 and 99; at least 29, 56 and 218; at least 56, 99 and 218; at least 29, 99 and 218; at least 99, 126 and 218), e.g. as presented in SEQ ID NO: 2. Said relevant or specific amino acids can be e.g. consensus or conserved amino acids. As used herein “a consensus amino acid” refers to an amino acid which is the one occurring most frequently at that amino acid site in the different sequences e.g., across species. As used herein “conserved amino acids” refers to identical or similar amino acids in polypeptides or proteins across species. Conservation indicates that an amino acid has been maintained by natural selection.

In one embodiment the enzyme or a fragment thereof comprises one or more relevant (e.g. consensus, conserved or catalytic) amino acids in specific positions selected from the group comprising or consisting of amino acid positions 29 (such as His29), 56 (such as Asp56), 59 (such as Gly59), 94 (such as Val94), 95 (such as Gly95), 97 (such as Ser97), 99 (such as Gly99), 126 (such as Pro126), 212 (such as Pro212), 218 (such as Gly218), 221 (such as Asp221), 249 (such as His249) and 266 (such as Phe266), and any combination thereof, e.g. as presented in SEQ ID NO: 2. In one embodiment the enzyme or a fragment thereof comprises one or more relevant amino acids in specific positions selected from the group comprising or consisting of amino acid positions 29 (e.g. His29), 56 (e.g. Asp56), 99 (e.g. Gly99), 126 (e.g. Pro126) and 218 (e.g. Gly218), e.g. as presented in SEQ ID NO: 2.

The enzyme of the present invention refers to not only fungal or bacterial but also any other enzyme homologue from any micro-organism, organism or mammal. Also, all isozymes, isoforms and variants are included with the scope of said enzyme. In one embodiment of the method, enzyme, fragment, micro-organism or host cell of the present invention, the enzyme originates from or is an enzyme of a bacterium or fungus selected from the group comprising or consisting of Bacillus, Paenibacillus, Achromobacter, Acinetobacter, Acidovorax, Alcanivorax, Aneurini-bacillus, Arthrobacter, Aspergillus, Brevibacillus, Chaetomium, Chitinophaga, Citrobacter, Cladosporium, Cupriavidus, Comamonas, Cordyceps, Cupriavidus, Delftia, Engyodontium, Enterobacter, Flavobacterium, Fusarium, Hyphomicrobium, Hypocrea, Klebsiella, Kocuria, Leucobacter, Lulwoana, Lysinibacillus, Macrococcus, Methylobacterium, Methylocella, Meyerozyma, Microbacterium, Micrococcus, Moraxella, Mortierella, Mucor, Nesiotobacter, Nocardia, Ochrobactrum, Oscillatoria, Pantoea, Paracoccus, Penicillium, Phanerochaete, Phormidium, Pleurotus, Pseudomonas, Rahnella, Ralstonia, Rhizobium, Rhodococcus, Sarocladium, Serratia, Staphylococcus, Stenotrophomonas, Streptococcus, Streptomyces, Trichoderma, Vibrio, Virgibacillus and Xanthobacter; or the enzyme is an enzyme of a bacterium or fungus selected from the group comprising or consisting of Achromobacter xylosoxidans, Acidovorax sp., Acinetobacter sp., Acinetobacter baumannii, Acinetobacter pittii, Alcanivorax borkumensis, Aneurinibacillus aneurinilyticus, Arthrobacter sp, Aspergillus acidus, Aspergillus awamori, Aspergillus brasiliensis, Aspergillus clavatus, Aspergillus flavus, Aspergillus foetidus, Aspergillus fumigatus, Aspergillus glaucus, Aspergillus japonicus, Aspergillus kawachii, Aspergillus nidulans, Aspergillus niger, Aspergillus nomius, Aspergillus oryzae, Aspergillus sp. Aspergillus sydowii, Aspergillus terreus, Aspergillus tubingensis, Aspergillus wentii, Bacillus amyloliquefaciens, Bacillus aryabhattai, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus gottheilii, Bacillus vallismortis, Bacillus vietnamensis, Brevibacillus sp., Brevibacillus brevis, Brevibacillus borstelensis, Brevibacillus agri, Brevibacillus parabrevis, Chaetomium sp., Chitinophaga sp., Citrobacter amalonaticus, Cladosporium cladosporioides, Comamonas sp., Cordyceps confragosa, Cupriavidus necator, Delftia sp., Delftia tsuruhatensis, Engyodontium album, Enterobacter sp., Enterobacter asburiare, Flavobacterium sp., Flavobacterium petrolei, Flavobacterium pectinovorum, Flavobacterium aquicola, Fusarium redolens, Fusarium solani, Fusarium sp., Fusarium oxysporum, Hyphomicrobium sp., Hypocrea sp., Klebsiella pneumoniae, Kocuria palustris, Leucobacter sp., Lulwoana uniseptata, Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Meyerozyma guilliermondii, Microbacterium sp., Microbacterium paraoxydans, Micrococcus sp., Micrococcus luteus, Micrococcus lylae, Moraxella sp., Mortierella alpina, Mucor circinelloides, Mucor sp., Nesiotobacter exalbescens, Nocardia asteroides, Ochrobactrum intermedium, Ochrobactrum oryzae, Oscillatoria subbrevis, Paenibacillus sp., Paenibacillus odorifer, Paenibacillus macerans, Pantoea sp., Penicillium digitatum, Penicillium chrysogenum, Penicillium glabrum, Penicillium oxalicum, Penisillium rubens, Penicillium simplicissimum, Penicillium sp., Paracoccus yeei, Phanerochaete chrysosporium, Phormidium lucidum, Pleurotus ostreatus, Pseudomonas aeruginosa, Pseudomonas azotoformans, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus gingshengii, Rhodococcus erythropolis, Rhodococcus rhodochrous, Rhodococcus ruber, Rhodococcus sp., Sarocladium kiliense, Serratia marcescens, Sphingobacterium multivorum, Staphylococcus sp., Staphylococcus epidermidis, Staphylococcus cohnii, Staphylococcus xylosus, Stenotrophomonas humi, Stenotrophomonas maltophilia, Stenotrophomonas panacihumi, Stenotrophomonas sp., Streptococcus sp., Streptomyces albogriseolus, Streptomyces badius, Streptomyces griseus, Streptomyces sp., Streptomyces viridosporus, Trichoderma harzianum, Trichoderma virens, Trichoderma viride, Vibrio alginolyticus, Vibrio parahaemolyticus, Virgibacillus halodenitrificans, Xanthobacter autotrophicus, and Xanthobacter tagetidis.

In one embodiment “an enzyme of a bacterium or fungus” refers to a situation, wherein the amino acid sequence of the enzyme has the same amino acid sequence as a wild type enzyme of a bacterium or fungus (e.g. any of the above listed bacteria or fungus) or the amino acid sequence of the enzyme has a high sequence identity (e.g. 50% or more, 60% or more, 70% or more, 80% or more, 85% or more, 90% or more, or 95% or more) to an amino acid sequence of a wild type bacterial or fungal enzyme (e.g. of any of the above listed bacteria or fungus). In other words, the amino acid sequence of the enzyme used in the present invention can be modified (e.g. genetically modified).

In one embodiment, the enzyme, fragment, micro-organism or host cell is a genetically modified enzyme, fragment, micro-organism or host cell. In a specific embodiment the enzyme, fragment, micro-organism or host cell has an increased ability to degrade a polyolefin compared to the corresponding unmodified enzyme, fragment, micro-organism or host cell, respectively. In one embodiment the enzyme, micro-organism or host cell comprises a genetic modification increasing an enzyme activity or the amount of a specific enzyme in a micro-organism or host cell. Genetic modifications (e.g. resulting in increased enzyme activity, increased expression of an enzyme, or increased or faster degradation of a polyolefin) include but are not limited to genetic insertions, deletions, disruptions or substitutions of one or more genes or a fragment(s) thereof or insertions, deletions, disruptions or substitutions of one or more nucleotides (e.g. insertion of a polynucleotide encoding an enzyme), or addition of plasmids. For example, one or several polynucleotides encoding an enzyme of interest can be integrated to the genome of a micro-organism or host cell. As used herein “disruption” refers to insertion of one or several nucleotides into a gene or polynucleotide sequence resulting in a lack of the corresponding polypeptide or enzyme or presence of non-functional polypeptide or enzyme with lowered activity. Methods for making genetic modifications or modifying micro-organisms or host cells (e.g. by adaptive evolution strategy) are generally well known by a person skilled in the art and are described in various practical manuals describing laboratory molecular techniques.

In one embodiment the enzyme or a fragment thereof has one or more genetic modifications (e.g. a targeted mutation or a modification by an adaptive evolution) after one or more amino acids corresponding to the amino acids selected from the group comprising or consisting of Ser97, Asp221, His249, His29, Asp56, Gly99, Pro126 and Gly218 presented in SEQ ID NO: 2. As used herein “after one or more amino acids” refers to immediately after said amino acid(s) e.g. a modification at least in the next amino acid or later after said amino acid (e.g. 1-50 amino acids, 1-30 amino acids, 1-20 amino acids, 1-10 amino acids or 1-5 amino acids after the specific amino acid mentioned above in the list of this paragraph).

As used herein “increased degradation (activity/ability/capability) of a polyolefin” or “faster degradation (activity/ability/capability) of a polyolefin” of an enzyme or micro-organism refers to the presence of higher activity or more activity of an enzyme or micro-organism, when compared to another enzyme or micro-organism, e.g. a genetically unmodified (wild type) enzyme or micro-organism. “Increased or faster degradation” may result e.g. from the presence of a specific enzyme in a micro-organism or an up-regulated gene or polypeptide expression in a micro-organism or an increased secretion of an enzyme by a micro-organism. Also, “increased or faster degradation” may result e.g. from the presence of (enhancing) mutations of a specific enzyme having degradation capability.

As used herein “up-regulation of the gene or polypeptide expression” refers to excessive expression of a gene or polypeptide by producing more products (e.g. mRNA or polypeptide, respectively) than an unmodified micro-organism. For example, one or more copies of a gene or genes may be transformed to a cell (e.g. to be integrated to the genome of the cell) for upregulated gene expression. The term also encompasses embodiments, where a regulating region such as a promoter or promoter region has been modified or changed or a regulating region (e.g. a promoter) not naturally present in the micro-organism has been inserted to allow the over-expression of a gene. Also, epigenetic modifications such as reducing DNA methylation or histone modifications as well as classical mutagenesis are included in “genetic modifications”, which can result in an upregulated expression of a gene or polypeptide. As used herein “increased or up-regulated expression” refers to an increased expression of the gene or polypeptide of interest compared to a wild type micro-organism without the genetic modification. Expression or increased expression can be proved for example by western, northern or southern blotting or quantitative PCR or any other suitable method known to a person skilled in the art. As used herein “increased secretion of an enzyme by a micro-organism” refers to a secretion of an enzyme outside of a cell, which produces said enzyme. Increased secretion may be caused e.g. by an increased or up-regulated expression of the gene or polypeptide of interest or by improved secretion pathway of the cell or molecules participating in the secretion of said enzyme. In one embodiment secretion of an enzyme can be increased by adding one or more glycosylation sites to the enzyme or by altering or deleting one or more glycosylation sites.

In one embodiment the genetically modified enzyme, micro-organism, host cell or polynucleotide is a recombinant enzyme, micro-organism, host cell or polynucleotide. As used herein, “a recombinant enzyme, micro-organism, host cell or polynucleotide” refers to any enzyme, micro-organism, host cell or polynucleotide that has been genetically modified to contain different genetic material compared to the enzyme, micro-organism, host cell or polynucleotide before modification (e.g. comprise a deletion, substitution, disruption or insertion of one or more nucleic acids or amino acids e.g. including an entire gene(s) or parts thereof). The recombinant micro-organism or host cell may also contain other genetic modifications than those specifically mentioned or described in the present disclosure. Indeed, the micro-organism or host cell may be genetically modified to produce, not to produce, increase production or decrease production of e.g. other polynucleotides, polypeptides, enzymes or compounds than those specifically mentioned in the present disclosure. In certain embodiments, the genetically modified micro-organism or host cell includes a heterologous polynucleotide or enzyme. The micro-organism or host cell can be genetically modified by transforming it with a heterologous polynucleotide sequence that encodes a heterologous polypeptide. For example, a cell may be transformed with a heterologous polynucleotide encoding an enzyme of the present invention either without a signal sequence or with a signal sequence. Alternatively, for example heterologous promoters or other regulating sequences can be utilized in the micro-organisms, host cells or polynucleotides of the invention. As used herein “a heterologous polynucleotide or enzyme” refers to a polynucleotide or enzyme, which does not naturally occur in a cell or micro-organism. In one embodiment of the present invention, the enzyme or fragment thereof is encoded by a heterologous polynucleotide sequence and optionally expressed by a micro-organism or host cell.

Genetic modifications may be carried out using conventional molecular biological methods. Genetic modification (e.g. of an enzyme or micro-organism) can be accomplished in one or more steps via the design and construction of appropriate vectors and transformation of the micro-organism cell with those vectors. For example, electroporation, protoplast-PEG and/or chemical (such as calcium chloride or lithium acetate based) transformation methods can be used. Also, any commercial transformation methods are appropriate. Suitable transformation methods are well known to a person skilled in the art.

The term “vector” refers to a nucleic acid compound and/or composition that transduces, transforms, or infects a micro-organism or a host cell, thereby causing the cell to express polynucleotides and/or proteins other than those native to the cell, or in a manner not native to the cell. An “expression vector” contains a sequence of nucleic acids to be expressed by the modified micro-organism. Optionally, the expression vector also comprises materials to aid in achieving entry of the nucleic acids into the micro-organism, such as a virus, liposome, protein coating, or the like. The expression vectors contemplated for use in the present invention include those into which a nucleic acid sequence (i.e. polynucleotide) can be inserted, along with any preferred or required operational elements. Further, the expression vector must be one that can be transferred into a micro-organism or host cell and replicated therein. Vectors can be circularized or linearized and may contain restriction sites of various types for linearization or fragmentation. In specific embodiments expression vectors are plasmids, particularly those with restriction sites that have been well documented and that contain the operational elements preferred or required for transcription of the nucleic acid sequence. Such plasmids, as well as other expression vectors, are well known to those of ordinary skill in the art. Useful vectors may for example be conveniently obtained from commercially available micro-organism, yeast or bacterial vectors. Successful transformants can be found or selected using the attributes contributed by the marker or selection gene. Screening can be performed e.g. by PCR or Southern analysis to confirm that the desired genetic modifications (e.g. deletions, substitutions or insertions) have taken place, to confirm copy number or to identify the point of integration of nucleic acids (i.e. polynucleotides) or genes into the micro-organism cell's genome. SDS page could be used for confirming that the polypeptide of interest has been produced.

Indeed, the present invention also relates to a polynucleotide encoding the enzyme of the present invention or a fragment thereof, and an expression vector or plasmid comprising said polynucleotide of the present invention.

In a specific embodiment the enzyme or an enzymatically active fragment or variant thereof comprises or has a sequence having at least 20%, 25%, 30%, 35%, 40%, 45%, 50%, 55%, 60%, 65%, 70%, 71%, 72%, 73%, 74%, 75%, 76%, 77%, 78%, 79%, 80%, 80.5%, 81%, 81.5%, 82%, 82.5%, 83%, 83.5%, 84%, 84.5%, 85%, 85.5%, 86%, 86.5%, 87%, 87.5%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99% (e.g. 99.1%, 99.2%, 99.3%, 99.4%, 99.5%, 99.6%, 99.7%, 99.8% or 99.9%) or 100% sequence identity to SEQ ID NO: 2, 4, 6, or 8. Said enzyme can be genetically modified (i.e. differs from the wild type enzyme) or unmodified. In a specific embodiment an enzyme is an isolated enzyme.

In one embodiment of the invention the enzyme has at least 20, 25, 26, 27, 28, 29, 30, 35, 40, 45, 50, 55, 60, 65, 70, 71, 72, 73, 74, 75, 76, 77, 78, 79, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99 (e.g. 99.1, 99.2, 99.3, 99.4, 99.5, 99.6, 99.7, 99.8 or 99.9%), or 100% sequence identity to SEQ ID NO: 2 (SEQ ID NO: 2 is a Bacillus cereus chloroperoxidase amino acid sequence).

In one embodiment, the enzyme or fragment comprises a signal sequence, e.g. a heterologous signal sequence or a signal sequence of an exogenous host cell producing said enzyme of a fragment thereof. The signal sequence can be located e.g. after or before the amino acid sequence of the enzyme e.g. for secreting said enzyme outside of the cell. The signal sequence can be any signal sequence i.e. a short polypeptide present at the N-terminus of synthesized polypeptides that are destined towards the secretory pathway, said polypeptides including but not limited to those polypeptides that are targeted inside specific organelles, secreted from the cell, or inserted into cellular membranes. In one embodiment the enzyme or fragment thereof comprises a signal sequence, does not comprise a detectable signal sequence, is secreted out of the cell which produces it, and/or is not secreted out of the cell which produces it. In one embodiment the enzyme or fragment thereof does not comprise a detectable signal sequence and is secreted out of the cell which produces it. In one embodiment the enzyme or fragment thereof comprises one or more glycosylation sites wherein glycosylation in said sites affects (e.g. increases or decreases) secretion of the enzyme out of the cell.

A polynucleotide of the present invention encodes the enzyme of the present invention or a fragment thereof. In a specific embodiment the polynucleotide comprises a sequence having a sequence identity of at least 15%, 20%, 25%, 30%, 35%, 40%, 45%, 50%, 55%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% to SEQ ID NO: 1, 3, 5, 7, or 17, or a variant thereof. Said polynucleotide can be genetically modified (i.e. differs from the wild type polynucleotide) or unmodified. In a specific embodiment the polynucleotide is an isolated polynucleotide.

Identity of any sequence or fragments thereof compared to the sequence of this disclosure refers to the identity of any sequence compared to the entire sequence of the present invention. As used herein, the % identity between the two sequences is a function of the number of identical positions shared by the sequences (i.e. % identity=# of identical positions/total # of positions×100), taking into account the number of gaps, and the length of each gap, which need to be introduced for optimal alignment of the two sequences. The comparison of sequences and determination of identity percentage between two sequences can be accomplished using mathematical algorithms available in the art. This applies to both amino acid and nucleic acid sequences. As an example, sequence identity may be determined by using BLAST (Basic Local Alignment Search Tools) or FASTA (FAST-All). In the searches, setting parameters “gap penalties” and “matrix” are typically selected as default. In one embodiment the sequence identity is determined against the full length sequence of the present disclosure.

Nucleic acid and amino acid databases (e.g., GenBank) can be used for identifying a polypeptide having an enzymatic activity or a polynucleotide sequence encoding said polypeptide. Sequence alignment software such as BLASTP (polypeptide), BLASTN (nucleotide) or FASTA can be used to compare various sequences. Briefly, any amino acid sequence having some homology to a polypeptide having enzymatic activity, or any nucleic acid sequence having some homology to a sequence encoding a polypeptide having enzymatic activity can be used as a query to search e.g. GenBank. Percent identity of sequences can conveniently be computed using BLAST software with default parameters. Sequences having an identities score and a positive score of a given percentage, using the BLAST algorithm with default parameters, are considered to be that percent identical or homologous.

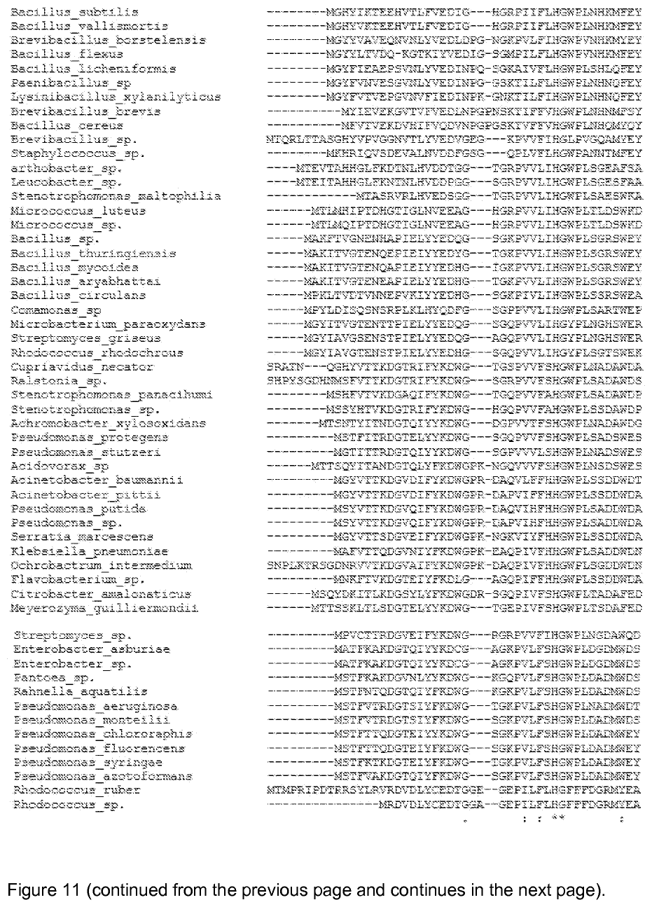

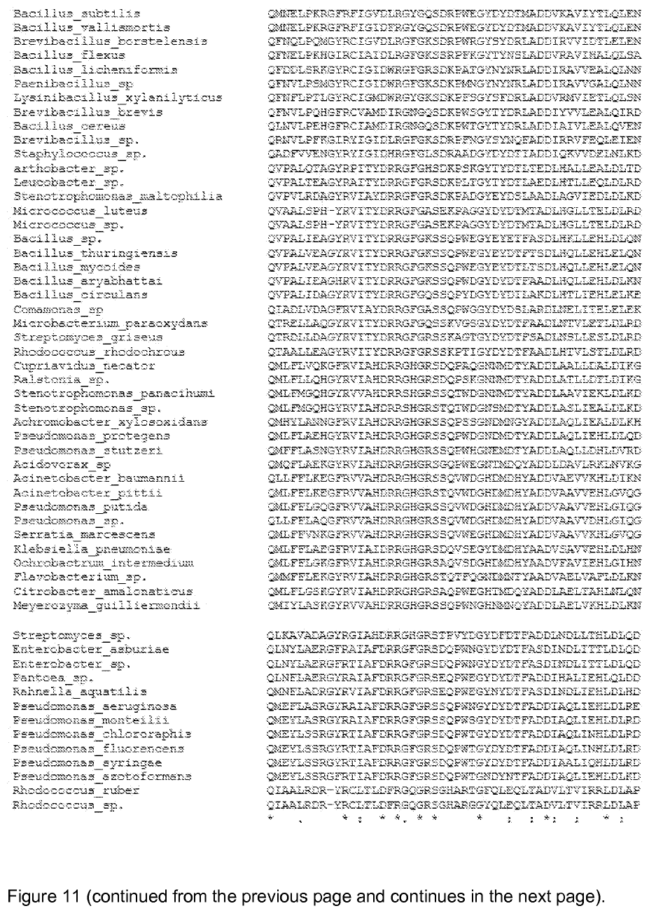

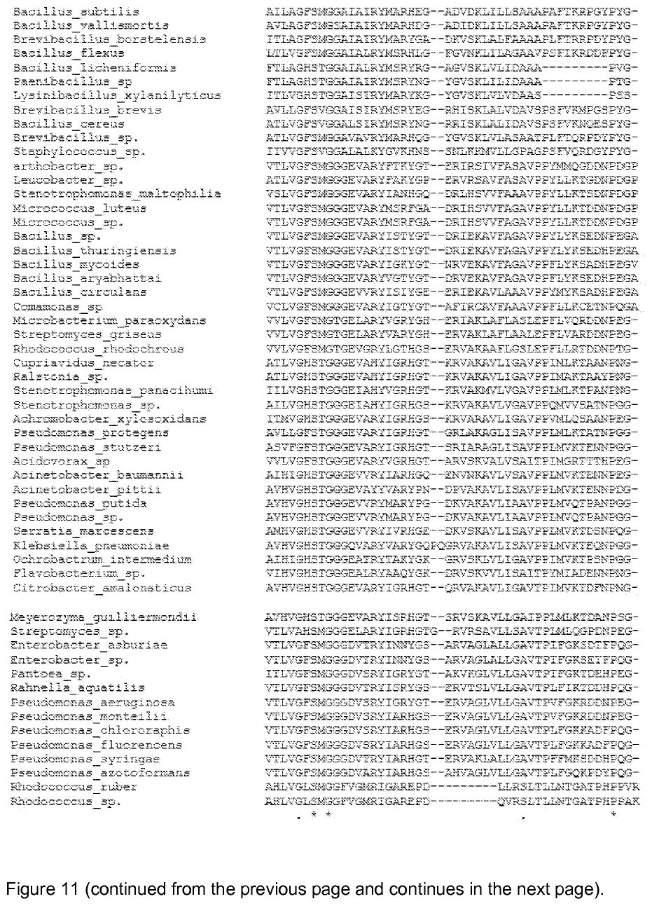

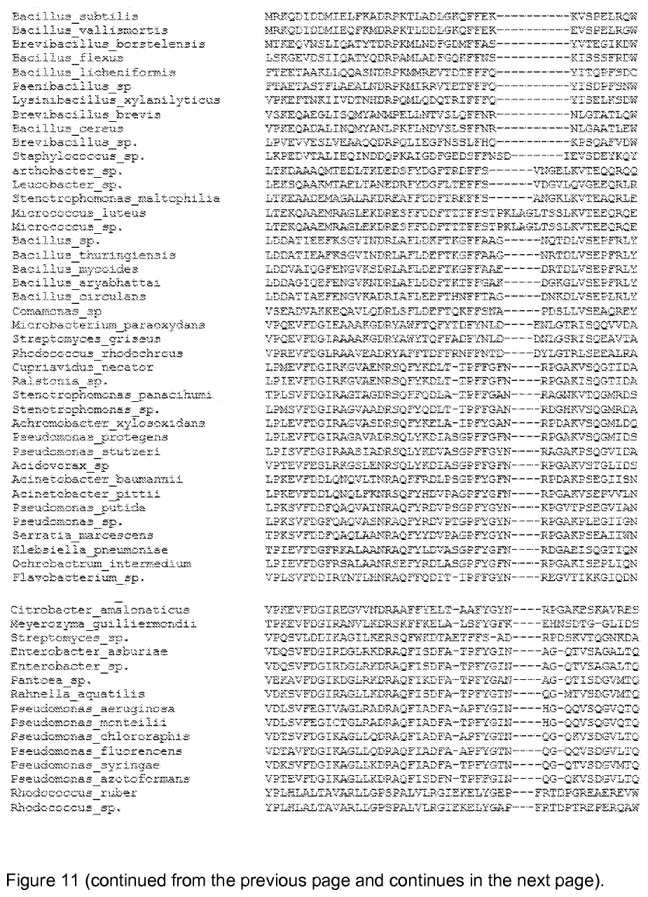

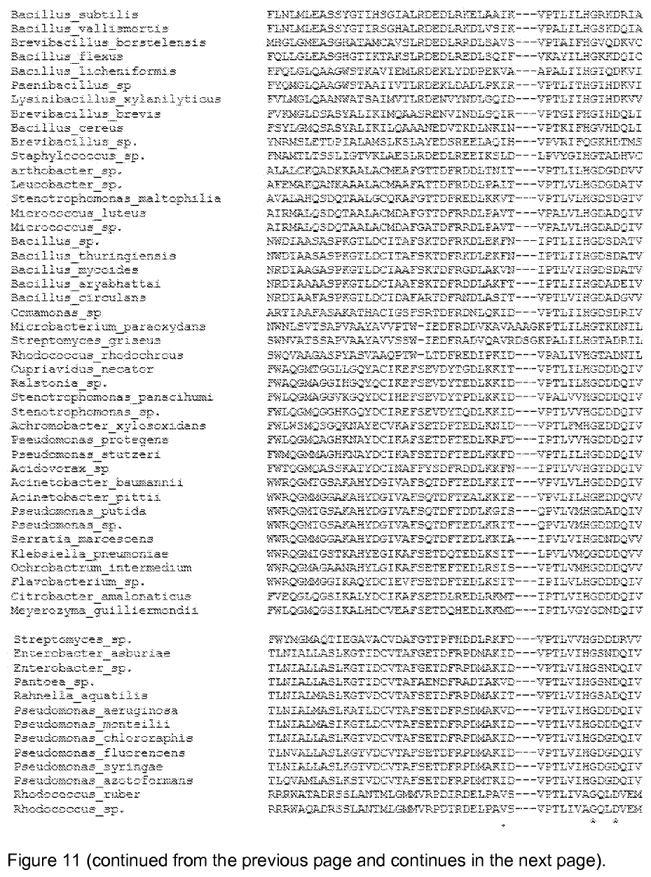

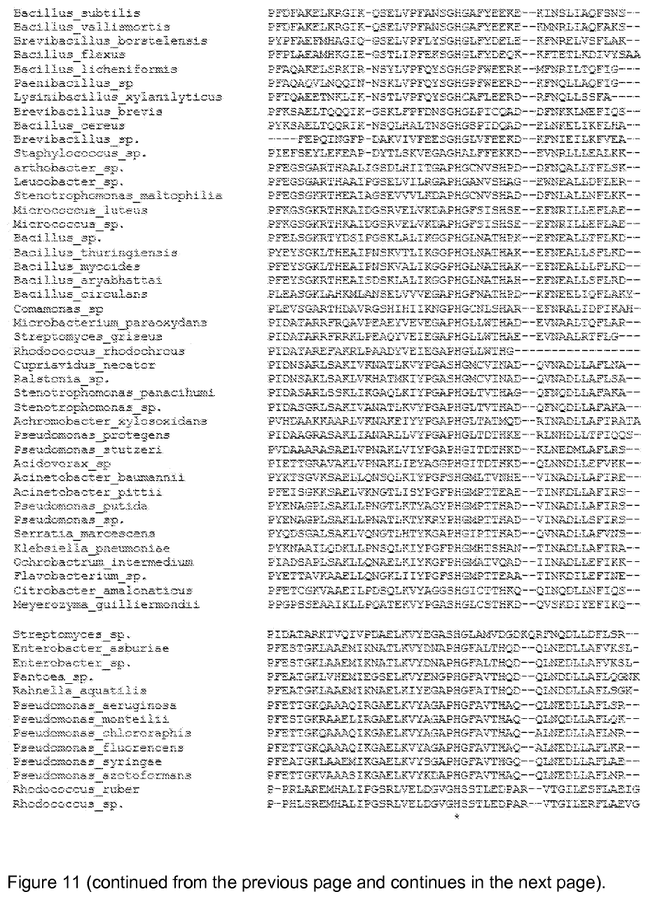

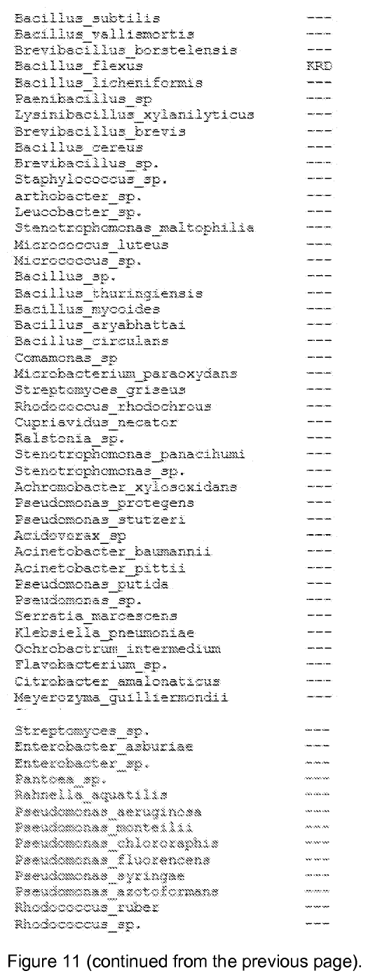

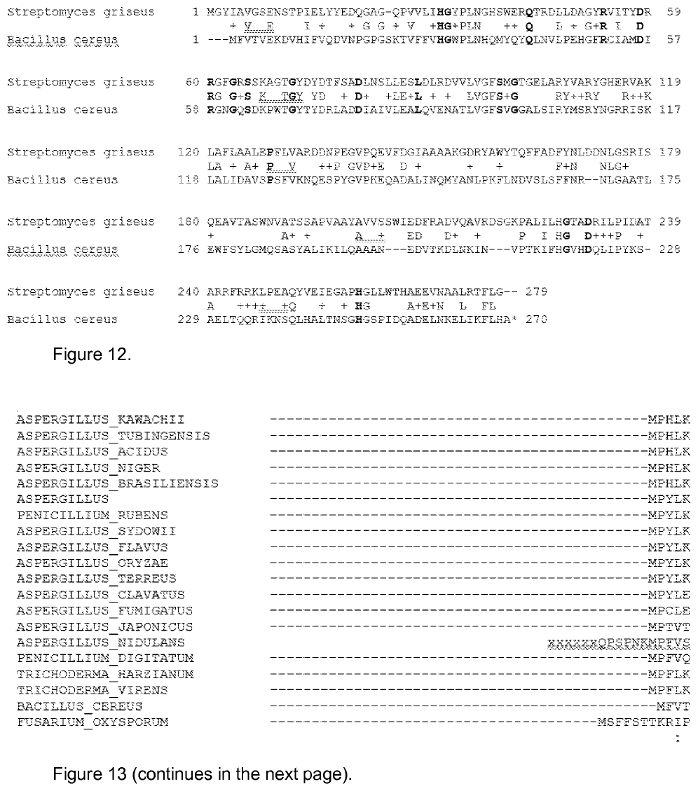

For example, an enzyme comprising a polyolefin degrading activity and e.g. comprising a catalytic triad Ser-Asp-His corresponding to amino acids Ser97, Asp221 and His249 presented in SEQ ID NO: 2, can be found as described in example 5. First, sequences containing similar kind of motifs can be searched e.g. with HMMER. HMMER is used for searching sequence databases for sequence homo-logs, and for making sequence alignments. It implements methods using probabilistic models called profile hidden Markov models (profile HMMs) (Robert D. Finn, Jody Clements, Sean R. Eddy (2011) HMMER web server: interactive sequence similarity searching. Nucleic Acids Research, Volume 39, Issue suppl_2, 1 Jul. 2011, Pages W29-W37, https://doi.org/10.1093/nar/gkr367). With the detected amino acid sequences or part of them or amino acid sequence(s) of previously known enzyme(s) sequence similarity searches against SEQ ID NO: 2 can be carried out e.g. by sequence alignment with ClustalW programme (https://www.genome.jp/tools-bin/clustalw) to detect corresponding consensus amino acids and their positions in amino acid sequence of interest. (See e.g. FIG. 11 or 12.)

In one embodiment, one of more of the consensus amino acids GLy99, Pro126 and Gly218 corresponding to amino acids as presented in SEQ ID NO: 2 were identified to have effect on the enzyme activity in addition to the amino acids Ser97, Asp221 and His249. According to the 3D and 2D structures these amino acids were predicted to be located in the loop area (Ser97, Pro126, Gly218, Asp221 and His249) and in alfa helix 3 (Gly99). The 3D structure of the enzyme and positions of beta sheets and alfa helixes (2D structure) can be predicted e.g. with Phyre2 protein homology/analogy engine recognition V 2.0 (www.sbq.bio.ic.ac.uk/phyre2/html/page.cgi?id=index). The 3D and 2D structures of proteins showing the alfa helixes and beta sheets can used in predicting and finding the amino acids important for the activity of the protein. The position of critical amino acids can be localised from the predicted 2D and 3D structures as described in Example 7.

In one embodiment one or more of the amino acids Ser97, Asp221 and His249, and optionally one or more of the amino acids His29, Asp56, Gly99, Pro126 and Gly218 (corresponding to the amino acid positions presented in SEQ ID NO: 2) are critical for the activity of the enzyme, e.g. degradation of a substrate (see FIG. 15. The enzyme can comprise one or more specific amino acids or amino acid motifs for example affecting a polyolefin degrading activity (e.g. enabling different substrates, binding of metal ions, and/or binding of hydrogen peroxide). In one embodiment one or more of the consensus amino acids affect the degrading activity (e.g. by increasing the degrading activity) of a polyolefin (e.g. Gly99, Pro126), increase possible interaction with substrates (e.g. Gly218), or affect binding of hydrogen peroxide (e.g. His29).

In one embodiment of the method, enzyme, fragment, micro-organism or host cell of the present invention, the enzyme is selected from the group comprising or consisting of a hydrolase, perhydrolase, esterase, carboxylesterase, aryl esterase, enol-lactonase, aminopeptidase, serine protease, lipase, epoxide hydrolase, dehalogenase, C—C hydrolase, lyase, dioxygenase, peroxidase, chloroperoxidase, bromoperoxidase, haloperoxidase and non-heme chloroperoxidase; and/or the enzyme comprises hydrolase, perhydrolase, esterase, carboxylesterase, aryl esterase, enol-lactonase, aminopeptidase, serine protease, lipase, epoxide hydrolase, dehalogenase, C—C hydrolase, lyase, dioxygenase, peroxidase, chloroperoxidase, bromoperoxidase, haloperoxidase or non-heme chloroperoxidase activity, or any combination thereof. In one embodiment of the method, enzyme, fragment, micro-organism or host cell of the present invention, the enzyme is selected from the group comprising or consisting of a hydrolase, esterase, aryl esterase, aminopeptidase, chloroperoxidase and bromoperoxidase; and/or the enzyme comprises hydrolase, esterase, aryl esterase, aminopeptidase, chloroperoxidase or bromoperoxidase activity, or any combination thereof. In one embodiment of the method, enzyme, fragment, micro-organism or cell of the present invention, the enzyme is selected from the group comprising or consisting of amino acid sequences identified in SEQ ID NO:90, SEQ ID NO:92, SEQ ID NO:94, SEQ ID NO:96, SEQ ID NO:98 or SEQ ID NO: 100. In one embodiment the enzyme or a fragment thereof comprises at least two different activities such as esterase and peroxidase (e.g. chloroperoxidase, bromoperoxidase, haloperoxidase or non-heme chloroperoxidase) activities.

As used herein “a hydrolase” refers to an enzyme which is capable of catalyzing the hydrolysis of a chemical bond optionally resulting in a degradation of a larger molecule into smaller molecules. Examples of hydrolases include but are not limited to esterases, lipases, phosphatases, glycosidases and peptidases. “An esterase” is a hydrolase enzyme that splits esters into an acid and an alcohol in a chemical reaction with water (i.e. hydrolysis). “Carboxylesterase” is responsible for the hydrolysis of carboxylesters into the corresponding alcohol and carboxylic acid. “Aryl esterase” catalyzes a chemical reaction wherein two substrates of the enzyme are phenyl acetate and water and two products from the chemical reaction are phenol and acetate. “Enol-lactonase” catalyzes the chemical reaction of enol-lactone and water. “Perhydrolase” refers to an enzyme that is capable of catalyzing a reaction with hydrogen peroxide and said reaction results in the formation of peracid. In one embodiment, the perhydrolase produces high perhydrolysis to hydrolysis ratios. “Perhydrolysis to hydrolysis ratio” is the ratio of the amount of enzymatically produced peracid to that of enzymatically produced acid by the perhydrolase, under defined conditions and within a defined time. “Aminopeptidase” catalyzes the cleavage of amino acids from the amino terminus (N-terminus) of proteins or peptides (exopeptidases). “Serine protease” cleaves peptide bonds in proteins, in which serine serves as the nucleophilic amino acid at the enzyme's active site. “Lipase” catalyzes the hydrolysis of lipids. “Epoxide hydrolase” metabolizes compounds that comprise an epoxide residue. Epoxide residue is converted to two hydroxyl residues through a dihydroxylation reaction to form diol products. “Dehalogenase” catalyzes the removal of a halogen atom from a substrate. “C—C hydrolase” cleaves a C—C bond. “Lyase” catalyzes the breaking of various chemical bonds by means other than hydrolysis and oxidation, often forming a new double bond or a new ring structure. “Dioxygenase” catalyzes reactions in which both atoms of molecular oxygen are incorporated into substrates.

“Peroxidases” are enzymes that catalyze oxidation-reduction reaction and break down hydrogen peroxide. “Haloperoxidases” mediate the oxidation of halides by hydrogen peroxide. “Chloroperoxidases” mediate oxidation of halides Cl⁻, Br⁻, and I⁻, and “bromoperoxidases” mediate oxidation of halides Br⁻ and I⁻. Chloroperoxidases can contain a heme or do not contain a heme (non-heme chloroperoxidases).

The enzyme(s) of fragments thereof involved in the degradation of polyolefins can be selected e.g. from one or several of the following: a hydrolase (EC 2.X, EC 3.X, EC 4.X), a metal dependent hydrolase (e.g. EC 3.1, EC 3.4 or EC 3.5), perhydrolase, esterase (EC 3.1.1.1), aryl esterase (EC 3.1.1.2), enol-lactonase (EC 3.1.1.24), aminopeptidase (EC 3.4.11.5), serine protease (EC 3.4.21.X), lipase (EC 3.1.1.3), epoxide hydrolase (EC 3.3.2.3), C—C hydrolase, dehalogenase (EC 3.8.1.5), lyase (EC 4.1.2.X), dioxygenase (EC 1.13.11.X), peroxidase (EC 1.11.1.7), chloroperoxidase (EC 1.11.1.10), bromoperoxidase (EC 1.11.1.18), haloperoxidase (EC 1.11.2.1), non-heme chloroperoxidase (EC 1.11.1.10), and any combination thereof.

In one embodiment the enzyme, fragment, micro-organism or host cell is capable of degrading the polyolefin in the presence or absence of hydrogen peroxide, such as added hydrogen peroxide. In one embodiment the enzyme, fragment, micro-organism or host cell of the present invention has produced hydrogen peroxide by itself. In another embodiment hydrogen peroxide has been produced by another enzyme, fragment, micro-organism or host cell, such as a glyoxal oxidase or a fragment thereof, or a micro-organism or host cell comprising said glyoxal oxidase. In one embodiment, hydrogen peroxide has been produced by a superoxide dismutase or a fragment thereof, or a micro-organism or host cell comprising said superoxide dismutase. Accordingly, in one embodiment, the enzyme, fragment, micro-organism or host cell of the present invention degrades polyolefin in the presence of hydrogen peroxide producing enzyme or a fragment thereof, or a micro-organism or host cell comprising said enzyme, such as glyoxal oxidase or superoxide dismutase.

In one embodiment, the enzyme, fragment, micro-organism or host cell capable of degrading polyolefin according to the present invention can be used together with micro-organisms that naturally degrade polyolefins slowly. The overexpression of an enzyme of the present invention seems to accelerate the degradation rate of polyolefins of such a micro-organism. In one embodiment, this also leads to an increased production of polyolefin degradation products, such as PHA, by the micro-organism naturally degrading polyolefins.

In one embodiment the enzyme and/or micro-organism have been genetically modified and optionally have an increased ability to degrade a polyolefin compared to the corresponding unmodified enzyme and/or micro-organism, respectively.

The presence, absence or amount of specific enzyme activities can be detected by any suitable method known in the art. Specific examples of studying enzyme activities of interest are well known to a person skilled in the art. Non-limiting examples of suitable detection methods include commercial kits on market, enzymatic assays, immunological detection methods (e.g., antibodies specific for said proteins), PCR based assays (e.g., qPCR, RT-PCR), and any combination thereof.

In one embodiment, the enzymes of the present invention have high turnover rates when degrading one or more polyolefins, e.g. when compared to prior art enzymes. In specific embodiments the activity of an enzyme to degrade a polyolefin is determined by an enzyme assay wherein said enzyme is allowed to contact with polyolefins (e.g. as described in example 2 or 4). In some embodiments the activity of an enzyme to degrade polyolefins can be determined e.g. by detecting or measuring the degradation products of polyolefins (e.g. as shown in example 2 or 3) or by analyzing the remaining starting material containing polyolefins after contacting the starting material with the enzymes.

Degradation of polyolefins can be measured by any suitable method known in the field. In one embodiment polyolefins or a material comprising polyolefins are weighed before and/or after said polyolefins or material have been contacted with an enzyme, micro-organism or host cell (or any combination thereof). The presence, absence or level of degradation products of a polyolefin, e.g. degraded by an enzyme, micro-organism or host cell, can be detected or measured by any suitable method known in the art. Non-limiting examples of suitable detection and/or measuring methods include liquid chromatography, gas chromatography, mass spectrometry or any combination thereof (e.g. ESI-MS/MS, MaldiTof, RP-HPLC, GC-MS or LC-TOF-MS) of samples, optionally after cultivating a micro-organism or host cell e.g. 1-11 hours, 11-100 hours, or 100 hours-12 months (e.g. one, two, three, four, five, six, seven, eight, nine, ten or 11 months) or even longer in the presence of polyolefins or after allowing a micro-organism, polypeptide or enzyme to contact with polyolefins. Other examples of suitable detection and/or measuring methods (including methods of fractionating, isolating or purifying degradation products) include but are not limited to filtration, solvent extraction, centrifugation, affinity chromatography, ion exchange chromatography, electrophoresis, hydrophobic interaction chromatography, gel filtration chromatography, reverse phase chromatography, chromatofocusing, differential solubilization, preparative disc-gel electrophoresis, isoelectric focusing, HPLC, gel permeation chromatography (GPC), fourier-transform infrared spectroscopy (FT-IR), NMR and/or re-versed-phase HPLC.

For degradation, polyolefins or a material comprising polyolefins can be contacted with an enzyme, micro-organism or host cell (or any combination thereof) e.g. at a ratio, concentration and/or temperature for a time sufficient for the degradation of interest. Suitable time for allowing the enzyme, micro-organism, or host cell to degrade a hydrocarbon chain or hydrocarbon chains can be selected e.g. from the group consisting of 1, 2, 3, 4, 5, 6, 7, 8, 9, 10, 11, 12, 13, 14, 15, 16, 17, 18, 19, 20, 21, 22, 23, and 24 hours, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and 31 days, 1, 2, 3, 4, 5, 6, 7, 8, 9, 10, 11, and 12 weeks, and 1, 2, 3, 4, 5, 6, 7, 8, 9, 10, 11 and 12 months. The degradation may take place in liquid, semi-solid, moist or dry conditions. The degradation is conveniently conducted aerobically, microaerobically and/or anaerobically. If desired, specific oxygen uptake rate can be used as a process control. The degradation can be conducted continuously, batch-wise, feed batch-wise or as any combination thereof.

In one embodiment the enzyme(s), micro-organism(s) or host cell(s) can be utilized for degrading polyolefins e.g. at a temperature below 100° C. such as 15-95° C., 30-95° C. or 40-80° C. (e.g. 50° C.) In one embodiment the enzyme, fragment, micro-organism or host cell is capable of degrading a polyolefin at a temperature of at least 20° C., at least 25° C., at least 30° C., or at least 37° C. This indicates low energy need and therefore also moderate costs of the method.

In some embodiments of the invention an enzyme and/or enzymes (e.g. a combination of different enzymes) can produce material (e.g. degradation products (such as alkane) or modified material) for other enzymes or enzymes of other type(s) or micro-organisms to further degrade or modify said material (e.g. to fatty acids, PHA or diacids). On the other hand, in some embodiments of the invention a micro-organism, host cell, micro-organisms (e.g. a combination of different micro-organisms) or host cells can produce material (e.g. degradation products (such as alkane) or modified material) for micro-organisms of other type(s) or enzymes to further degrade or modify said material (e.g. to fatty acids, PHA or diacids). In some embodiments of the present invention the micro-organisms or host cells are cultured under conditions (e.g. suitable conditions) in which the cultured micro-organism or host cell produces polypeptides, enzymes or compounds or interest (e.g. enzymes for degrading polyolefins). The micro-organisms or host cells can be cultivated in a medium containing appropriate carbon sources together with other optional ingredients selected from the group consisting of nitrogen or a source of nitrogen (such as amino acids, proteins, inorganic nitrogen sources such as nitrate, ammonia, urea or ammonium salts), yeast extract, peptone, minerals and vitamins, such as KH₂PO₄, Na₂HPO, MgSO, CaCl₂), FeCl₂, ZnSO, citric acid, MnSO, COCl₂, CuSO, Na₂MoO₄, FeSO₄, HsBO₄, D-biotin, Ca-Pantothenate, nicotinic acid, myoinositol, thiamine, pyridoxine, p-amino benzoic acid. Suitable cultivation conditions, such as temperature, cell density, selection of nutrients, and the like are within the knowledge of a skilled person and can be selected to provide an economical process with the micro-organism in question. Temperatures may range from above the freezing temperature of the medium to about 50° C. or even higher, although the optimal temperature will depend somewhat on the particular micro-organism. In a specific embodiment the temperature is from about 25 to 35° C. The pH of the cultivation process may or may not be controlled to remain at a constant pH, but is usually between 3 and 9, depending on the production organism. Optimally the pH can be controlled e.g. to a constant pH of 7-8 (e.g. in the case of Escherichia coli) or to a constant pH of 5-6 (e.g. in the case of Yarrowia lipolytica). Suitable buffering agents include, for example, calcium hydroxide, calcium carbonate, sodium hydroxide, potassium hydroxide, potassium carbonate, hydrogen chloride, sodium carbonate, ammonium carbonate, ammonia, ammonium hydroxide and/or the like. In general, those buffering agents that have been used in conventional cultivation methods are also suitable here.

The micro-organisms or host cells can be normally separated from the culture medium after cultivation, before or after contacting with a polyolefin. The separated micro-organisms, host cells or a liquid (e.g. culture medium) comprising micro-organisms or host cells can be used for contacting polyolefins.

Polypeptides or enzymes can be secreted outside of the cells or they can stay in the cells. Therefore, the polypeptides or enzymes can be recovered from the cells or directly from the culture medium. In some embodiments both intracellular and extracellular polypeptides or enzymes are recovered. Prior to recovering, cells can be disrupted. Isolation and/or purification of polypeptides or enzymes can include one or more of the following: size exclusion, desalting, anion and cation exchange, based on affinity, removal of chemicals using solvents, extraction of the soluble proteinaceous material e.g. by using an alkaline medium (e.g. NaOH, Borate-based buffers or water is commonly used), isoelectric point-based or salt-based precipitation of proteins, centrifugation, and ultrafiltration. In one embodiment of the method, polypeptide or enzyme of the present invention, said polypeptide or enzyme is a purified or partly purified polypeptide or enzyme. If the polypeptide or enzyme is secreted outside of the cell it does not necessarily need to be purified.

In one embodiment, the enzyme or fragment thereof is immobilized. Immobilization can be carried out by any method known to a person skilled in the art such as immobilization by crosslinking e.g. with glutaraldehyde.

Polyolefin(s) degrading enzymes can be expressed in any suitable host (cell). Examples of suitable host cells include but are not limited to cells of micro-organisms such as bacteria, yeast, fungi and filamentous fungi, as well as cells of plants and animals (such as mammals). In one embodiment the host cell is selected from the group consisting of Escherichia, Yarrowia, Pichia, Saccharomyces, Trichoderma, Aspergillus, Bacillus, Myceliophthora, Escherichia coli, Yarrowia lipolytica, Pichia pastoris, Saccharomyces cerevisiae, Trichoderma reesei, Aspergillus nidulans, Aspergillus niger, Aspergillus oryzae, Bacillus licheniformis, Bacillus subtilis, and Myceliophthora thermophila.

In one embodiment of the invention the micro-organism(s) or host cell(s) is(are) a bacterium, bacteria, fungus or fungi selected from the group comprising or consisting of Bacillus, Paenibacillus, Achromobacter, Acinetobacter, Acidovorax, Alcanivorax, Aneurinibacillus, Arthrobacter, Aspergillus, Brevibacillus, Chaetomium, Chitinophaga, Citrobacter, Cladosporium, Cupriavidus, Comamonas, Cordyceps, Cupriavidus, Delftia, Engyodontium, Enterobacter, Flavobacterium, Fusarium, Hyphomicrobium, Hypocrea, Klebsiella, Kocuria, Leucobacter, Lulwoana, Lysinibacillus, Macrococcus, Methylobacterium, Methylocella, Meyerozyma, Microbacterium, Micrococcus, Moraxella, Mortierella, Mucor, Nesiotobacter, Nocardia, Ochrobactrum, Oscillatoria, Pantoea, Paracoccus, Penicillium, Phanerochaete, Phormidium, Pleurotus, Pseudomonas, Rahnella, Ralstonia, Rhizobium, Rhodococcus, Sarocladium, Serratia, Staphylococcus, Stenotrophomonas, Streptococcus, Streptomyces, Trichoderma, Vibrio, Virgibacillus and Xanthobacter, and any combination thereof; or the micro-organism(s) or host cell(s) is(are) a bacterium, bacteria, fungus or fungi selected from the group comprising or consisting of Achromobacter xylosoxidans, Acidovorax sp., Acinetobacter sp., Acinetobacter baumannii, Acinetobacter pittii, Alcanivorax borkumensis, Aneurinibacillus aneurinilyticus, Arthrobacter sp, Aspergillus acidus, Aspergillus awamori, Aspergillus brasiliensis, Aspergillus clavatus, Aspergillus flavus, Aspergillus foetidus, Aspergillus fumigatus, Aspergillus glaucus, Aspergillus japonicus, Aspergillus kawachii, Aspergillus nidulans, Aspergillus niger, Aspergillus nomius, Aspergillus oryzae, Aspergillus sp. Aspergillus sydowii, Aspergillus terreus, Aspergillus tubingensis, Aspergillus wentii, Bacillus amyloliquefaciens, Bacillus aryabhattai, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus gottheilii, Bacillus vallismortis, Bacillus vietnamensis, Brevibacillus sp., Brevibacillus brevis, Brevibacillus borstelensis, Brevibacillus agri, Brevibacillus parabrevis, Chaetomium sp., Chitinophaga sp., Citrobacter amalonaticus, Cladosporium cladosporioides, Comamonas sp., Cordyceps confragosa, Cupriavidus necator, Delftia sp., Delftia tsuruhatensis, Engyodontium album, Enterobacter sp., Enterobacter asburiare, Flavobacterium sp., Flavobacterium petrolei, Flavobacterium pectinovorum, Flavobacterium aquicola, Fusarium oxysporum, Fusarium redolens, Fusarium solani, Fusarium sp., Hyphomicrobium sp., Hypocrea sp., Klebsiella pneumoniae, Kocuria palustris, Leucobacter sp., Lulwoana uniseptata, Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Meyerozyma guilliermondii, Microbacterium sp., Microbacterium paraoxydans, Micrococcus sp., Micrococcus luteus, Micrococcus lylae, Moraxella sp., Mortierella alpina, Mucor circinelloides, Mucor sp., Nesiotobacter exalbescens, Nocardia asteroides, Ochrobactrum intermedium, Ochrobactrum oryzae, Oscillatoria subbrevis, Paenibacillus sp., Paenibacillus odorifer, Paenibacillus macerans, Pantoea sp., Penicillium digitatum, Penicillium chrysogenum, Penicillium glabrum, Penicillium oxalicum, Penicillium rubens, Penicillium simplicissimum, Penicillium sp., Paracoccus yeei, Phanerochaete chrysosporium, Phormidium lucidum, Pleurotus ostreatus, Pseudomonas aeruginosa, Pseudomonas azotoformans, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus gingshengii, Rhodococcus erythropolis, Rhodococcus rhodochrous, Rhodococcus ruber, Rhodococcus sp., Sarocladium kiliense, Serratia marcescens, Sphingobacterium multivorum, Staphylococcus sp., Staphylococcus epidermidis, Staphylococcus cohnii, Staphylococcus xylosus, Stenotrophomonas humi, Stenotrophomonas maltophilia, Stenotrophomonas panacihumi, Stenotrophomonas sp., Streptococcus sp., Streptomyces albogriseolus, Streptomyces badius, Streptomyces griseus, Streptomyces sp., Streptomyces viridosporus, Trichoderma harzianum, Trichoderma virens, Trichoderma viride, Vibrio alginolyticus, Vibrio parahaemolyticus, Virgibacillus halodenitrificans, Xanthobacter autotrophicus, and Xanthobacter tagetidis, and any combination thereof.

Also, the micro-organism or host cell of the present invention can be used in a combination with any other micro-organism (simultaneously or consecutively), e.g. micro-organisms can be a population of different micro-organisms degrading different polyolefins or micro-organisms can be a combination of at least one bacterium and at least one fungus (to be used simultaneously or consecutively).

The inventors of the present disclosure have been able to isolate enzymes capable of degrading polyolefins from micro-organisms, and use said enzymes or micro-organisms for degrading polyolefins and/or producing degradation products of interest.

The present invention further relates to use of the enzyme, micro-organism, host cell, polynucleotide, expression vector or plasmid of the present invention or any combination thereof for degrading a polyolefin or polyolefins of different types.

Also, the present invention concerns a method of producing the enzyme or a fragment thereof of the present invention, wherein a recombinant micro-organism or host cell comprising the polynucleotide encoding the enzyme or fragment thereof of the present invention expresses or is allowed to express said enzyme or fragment thereof. For example, a vector or plasmid comprising the polynucleotide of interest can be transfected to a host cell, and the host cell can be used for expressing the enzyme of the present invention. In one embodiment the polynucleotide of interest is integrated into the genome of the host cell or the polynucleotide of interest is expressed from a vector or plasmid which is not integrated into the genome of the host cell. In one embodiment said expression of the enzyme can be controlled for example through inducible elements of promoters, vectors or plasmids.

As used in the present disclosure, the terms “polypeptide” and “protein” are used interchangeably to refer to polymers of amino acids of any length. As used herein “an enzyme” refers to a protein or polypeptide which is able to accelerate or catalyze (bio)chemical reactions.

As used herein “polynucleotide” refers to any polynucleotide, such as single or double-stranded DNA (genomic DNA or cDNA or synthetic DNA) or RNA (e.g. mRNA or synthetic RNA), comprising a nucleic acid sequence encoding a polypeptide in question or a conservative sequence variant thereof. Conservative nucleotide sequence variants (i.e. nucleotide sequence modifications, which do not significantly alter biological properties of the encoded polypeptide) include variants arising from the degeneration of the genetic code and from silent mutations.

As used herein “isolated” enzymes, polypeptides or polynucleotides refer to enzymes, polypeptides or polynucleotides purified to a state beyond that in which they exist in cells. Isolated polypeptides, proteins or polynucleotides include e.g. substantially purified (e.g. purified to at least 10%, 20%, 30%, 40%, 50%, 60%, 70%, 80%, 90% purity) or pure enzymes, polypeptides or polynucleotides.

It is well known that a deletion, addition or substitution of one or a few amino acids of an amino acid sequence of an enzyme does not necessarily change the catalytic properties of said enzyme. Therefore, the invention also encompasses variants and fragments of the enzymes of the present invention or given amino acid sequences having the stipulated enzyme activity. The term “variant” as used herein refers to a sequence having minor changes in the amino acid sequence as compared to a given sequence. Such a variant may occur naturally e.g. as an allelic variant within the same strain, species or genus, or it may be generated by mutagenesis or other gene modification. It may comprise amino acid substitutions, deletions or insertions, but it still functions in substantially the same manner as the given enzymes, in particular it retains its catalytic function as an enzyme (e.g. capability to degrade a hydrocarbon chain). In one embodiment of the invention a fragment of the enzyme is an enzymatically active fragment or variant thereof.

A “fragment” of a given enzyme or polypeptide sequence means part of that sequence, e.g. a sequence that has been truncated at the N- and/or C-terminal end. It may for example be the mature part of an enzyme or polypeptide comprising a signal sequence, or it may be only an enzymatically active fragment of the mature enzyme or polypeptide.

It will be obvious to a person skilled in the art that, as the technology advances, the inventive concept can be implemented in various ways. The invention and its embodiments are not limited to the examples described below but may vary within the scope of the claims.

### EXAMPLES

### Example 1. Expression of Bacillus cereus, Bacillus Flexus, Bacillus Licheniformis and Rhodococcus ruber Chloroperoxidases in Escherichia coli

The gene encoding Bacillus cereus chloroperoxidase (SEQ ID NO: 2) amino acid was cloned from genomic Bacillus cereus DNA by PCR by using oligonucleotides oPlastBug-120 (ACAATTCCTCTAGAAATAATTTTGTTTAACTTTAAGAAGGAGATATATCCATGG CTTACGAATTACCACAATTACCTTATGCA, SEQ ID NO: 9) and oPlastBug-121 (TAGCAGCCGGATCAAGCTGGGATTTAGGTGACACTATAGAATACTCTTATGC ATGTAAAAATTTTATCAGTTCTTTATTTAATTCATCCG, SEQ ID NO: 10). The resulting DNA fragment containing coding region of the gene (SEQ ID NO: 1) was cloned into NcoI and HindIII digested Escherichia coli expression vector pBAT4 with Gibson assembly resulting in plasmid pPB036-7 and expressed in E. coli strain Shuffle T7 Express (New England Biolabs).

The gene encoding Bacillus flexus chloroperoxidase (SEQ ID NO: 4) amino acid was cloned from genomic Bacillus flexus DNA by PCR by using oligonucleotides oPlastBug-232 (TAGAAATAATTTTGTTTAACTTTAAGAAGGAGATATATCCATGGGATATTACCT TACCGTTGATCAAAAGGG, SEQ ID NO: 11) and oPlastBug-233 (CAAGCTGGGATTTAGGTGACACTATAGAATACTCAAGCTTTCAATCTCTTTTC GCAGCAGAATAAACAATGTC, SEQ ID NO: 12). The resulting DNA fragment containing coding region of the gene (SEQ ID NO: 3) was cloned into NcoI and HindIII digested Escherichia coli expression vector pBAT4 with Gibson assembly resulting in plasmid pPB088-4 and expressed in E. coli strain Shuffle T7 Express (New England Biolabs)

The gene encoding Bacillus licheniformis chloroperoxidase (SEQ ID NO: 6) amino acid was cloned from genomic Bacillus licheniformis DNA by PCR by using oligonucleotides oPlastBug-234 (TAGAAATAATTTTGTTTAACTTTAAGAAGGAGATATATCCATGGGATATTTTAT CGAGGCAGAACCAAGC, SEQ ID NO: 13) and oPlastBug-235 (CAAGCTGGGATTTAGGTGACACTATAGAATACTCAAGCTTTCAACCGATAAAT TGCGTTAAAATCCGATTGAAC, SEQ ID NO: 14). The resulting DNA fragment containing coding region of the gene (SEQ ID NO: 5) was cloned into NcoI and HindIII digested Escherichia coli expression vector pBAT4 with Gibson assembly resulting in plasmid pPB089-6 and expressed in E. coli strain Shuffle T7 Express (New England Biolabs).

The gene encoding Rhodococcus ruber chloroperoxidase (SEQ ID NO: 8) amino acid was cloned from genomic Rhodococcus ruber DNA by PCR by using oligonucleotides oPlastBug-236 (TAGAAATAATTTTGTTTAACTTTAAGAAGGAGATATATCCATGCGGGACGTCG ATCTGTACTG, SEQ ID NO: 15) and oPlastBug-237 (CAAGCTGGGATTTAGGTGACACTATAGAATACTCAAGCTTTCAGCCGATCTC GGCGAGGA, SEQ ID NO: 16). The resulting DNA fragment containing coding region of the gene (SEQ ID NO: 7) was cloned into NcoI and HindIII digested Escherichia coli expression vector pBAT4 with Gibson assembly resulting in plasmid pPB090-5 and expressed in E. coli strain Shuffle T7 Express (New England Biolabs).

Plasmid pPB036-7, pPB088-4, pPB089-6 and pPB090-5 were expressed in E. coli Shuffle T7 Express grown at +37° C. in SB (30 g tryptone, 20 g yeast extract, 10 g MOPS (3-[N-morpholino]-propanesulfonic acid) per liter) media containing 100 μg/ml ampicillin. Protein expression was induced by the addition of 1 mM B-D-1-thiogalactopyranoside (IPTG), and induced cultures were further incubated at +30° ° C. for 24 hours. Cells were harvested by centrifugation (3184 g, 10 min RT), and supernatant was collected and stored at −80° C. or was used directly in enzyme assays. The pellet was suspended in 100 mM Na-phosphate pH 7.0 and homogenised with glass beads with with Precellys homogenizator. After homogenisation samples were centrifuged (20817 g, 27 min at +4 C) and cell extracts were collected and stored at −80° C. or were used directly in enzyme assays.

### Example 2. Chloroperoxidase Activity with Bacillus cereus, Bacillus Flexus, Bacillus Licheniformis and Rhodococcus ruber Enzymes

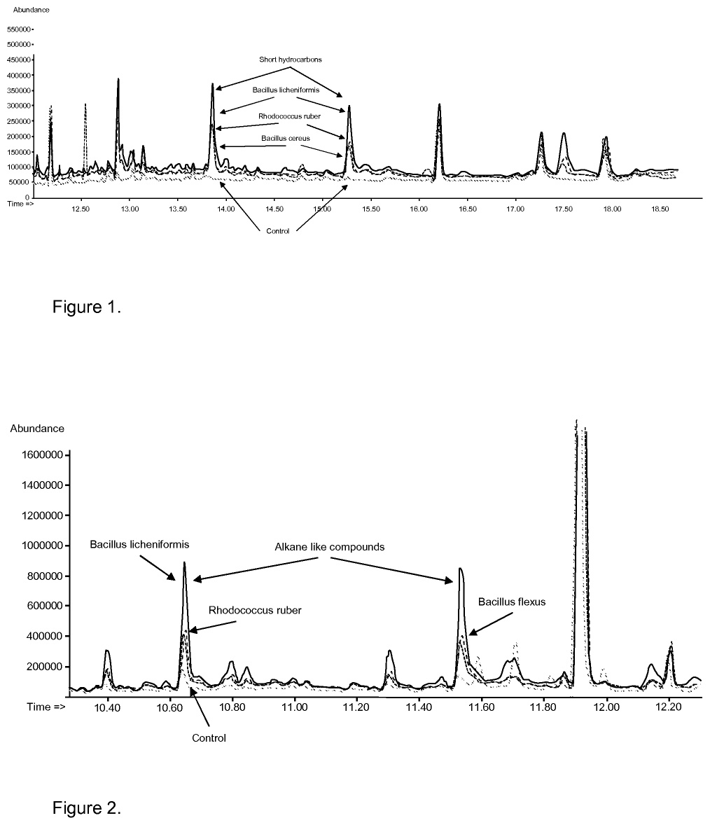

Enzyme assay with polyethylene with Bacillus cereus, Bacillus licheniformis and Rhodococcus ruber enzymes were carried out with cell extracts as follows: 1.2 ml of 50 mM HEPES pH 8.0 and 110 mM H₂O₂ with UV treated polyethylene powder (average MW˜4000 dalton, Sigma-Aldrich) was incubated with 50 μl of E. coli cell extracts from Example 1 at +30° C. for 118 hours. Polyethylene powder was UV treated with UV Stratalinker 2400 by using 9999×100 μJoule (254 nm). As a control cell extract from the culture with E. coli strain having empty plasmid (pBAT4) was used. After incubation GC-MS run was carried out with liquid fraction as described in Example 3. Results from the GC-MS run are shown in FIG. 1. With enzyme samples several peaks appeared which were missing from control sample. These peaks presented alkane like compounds or oxygen containing hydrocarbons.

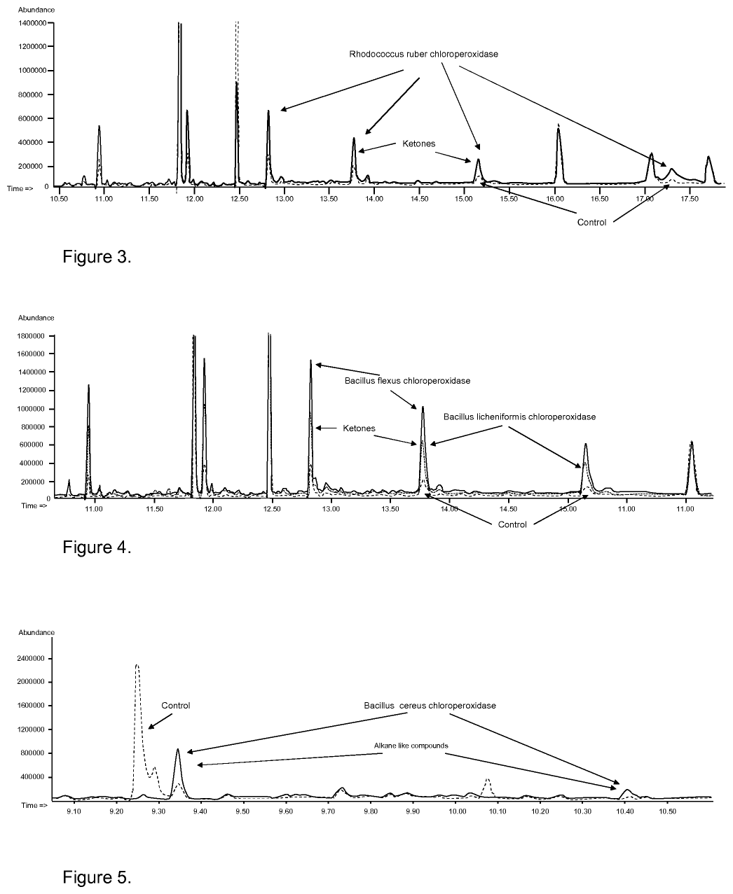

Enzyme assay with polypropylene with Bacillus flexus, Bacillus licheniformis and Rhodococcus ruber enzymes were carried out with cell extracts as follows: 1.2 ml of 150 mM Mcllvaine buffer pH 3.0 and 17 mM H₂O₂ with polypropylene powder (Licocene PP 6102 Fine grain, Clariant) was incubated with 50 μl of E. coli cell extracts from Example 1 at +30° C. for 118 hours. As a control cell extract from the culture with E. coli strain having empty plasmid (pBAT4) was used. After incubation GC-MS run was carried out with liquid fraction as described in Example 3. Results from the GC-MS run are shown in FIG. 2. With enzyme samples several peaks appeared which were missing from control sample. These peaks presented alkane like compounds.

Enzyme assay with polyethylene with Rhodococcus ruber enzyme was carried out with supernatant sample as follows: 1.2 ml of 150 mM Mcllvaine pH 6.0 and 17 mM H₂O₂ with polyethylene powder (average MW˜4000 dalton, Sigma-Aldrich) was incubated with 100 μl of E. coli supernatant sample from Example 1 at +28° C. for 112 hours. As a control supernatant from the culture with E. coli strain having empty plasmid (pBAT4) was used. After incubation GC-MS run was carried out with liquid fraction as described in Example 3. Results from the GC-MS run are shown in FIG. 3. With enzyme sample several peaks presenting ketones were higher in enzyme sample than in control sample. Additionally, peaks presenting longer alcohols were detected only with enzyme sample.

Enzyme assay without hydrogen peroxide with polyethylene with Bacillus flexus and Bacillus licheniformis enzymes were carried out with supernatant samples as follows: 1.2 ml of 150 mM Mcllvaine pH 6.0 with polyethylene powder (average MW˜4000 dalton, Sigma-Aldrich) was incubated with 100 μl of E. coli supernatant sample from Example 1 at +28° C. for 112 hours. As a control supernatant from the culture with E. coli strain having empty plasmid (pBAT4) was used. After incubation GC-MS run was carried out with liquid fraction as described in Example 3. Results from the GC-MS run are shown in FIG. 4. With enzyme samples several peaks presenting ketones were higher in enzyme samples than in control sample. Additionally, peaks presenting alkane like compounds were detected only with enzyme samples.

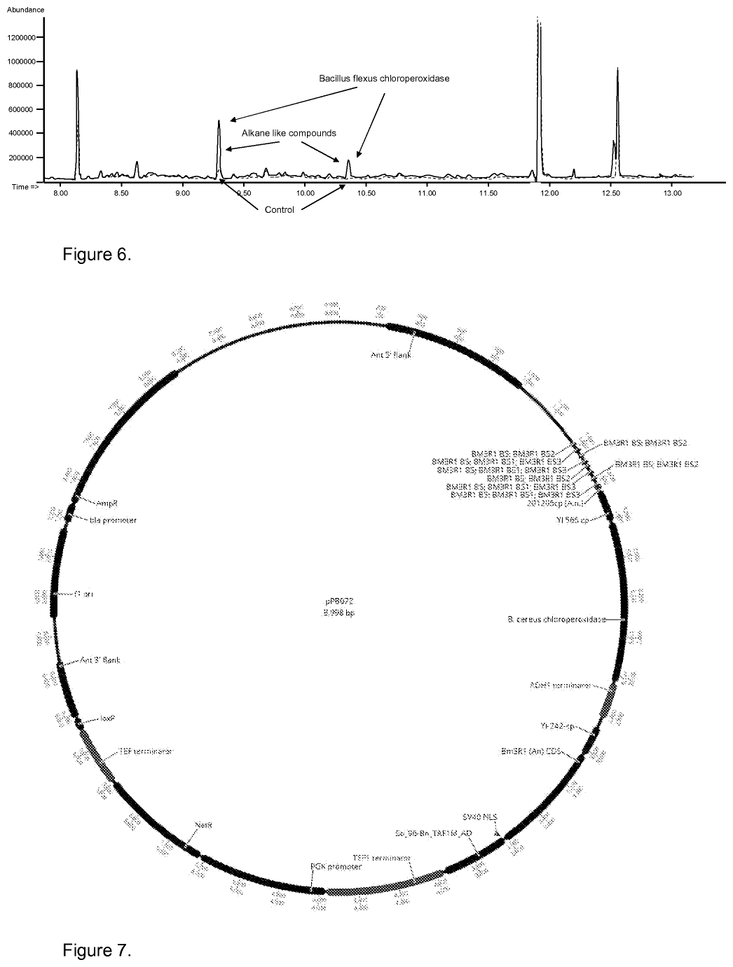

Enzyme assay with Bacillus cereus and Bacillus flexus enzymes with LDPE or HDPE film, respectively, were carried out as follows: 3.35 ml of 50 mM HEPES pH 8.0 and 17 mM H₂O₂ and LDPE film (Thickness 0.23 mm, additive free polymer, biaxially oriented, Goodfellow) or HDPE (Thickness 2 mm, Goodfellow) were incubated with 150 μl of E. coli supernatant samples from Example 1 at +28° C. with 800 rpm shaking for 145 hours. As a control supernatant from the culture with E. coli strain having empty plasmid (pBAT4) was used. After incubation SPME GC-MS run was carried out with liquid fraction as described in Example 3. Results from the GC-MS run are shown in FIGS. 5 and 6. With enzyme samples several peaks appeared which were missing from control samples. These peaks presented alkanes like compounds.

### Example 3. Gas Chromatography—Mass Spectrometry (GC-MS) Analysis of Volatile Degradation Products of Polyethylene with Chloroperoxidases

Aliquots (300 μl) of the samples from examples 2 and 4 were transferred to new tubes and an internal standard (methyl heptadecanoate) was added. The samples were extracted with dichloromethane (200 μl) by agitating in a shaker for 15 min. After extraction the samples were allowed settle (15 minutes at room temperature), centrifuged (5 min, 10 000 rpm) and finally, the dichloromethane phase was transferred to GC-MS vials. The runs were performed on Agilent GC-MS equipped with an HP-FFAP (25 m×200 μm×0.3 μm) column and helium was used as a carrier gas. The injector temperature was 250° C., and a splitless injection mode was used. The oven temperature was 40° C. for 3 min, increased to 240° C. at 20° C./min and kept at 240° C. for 14 min. The detected mass range was 35-600 m/z and compounds were identified based on NIST08 MS library. Results from GC-MS analysis are described in Examples 2 and 4.

### Example 4. Expression of Bacillus cereus Chloroperoxidase in Yarrowia Lipolytica

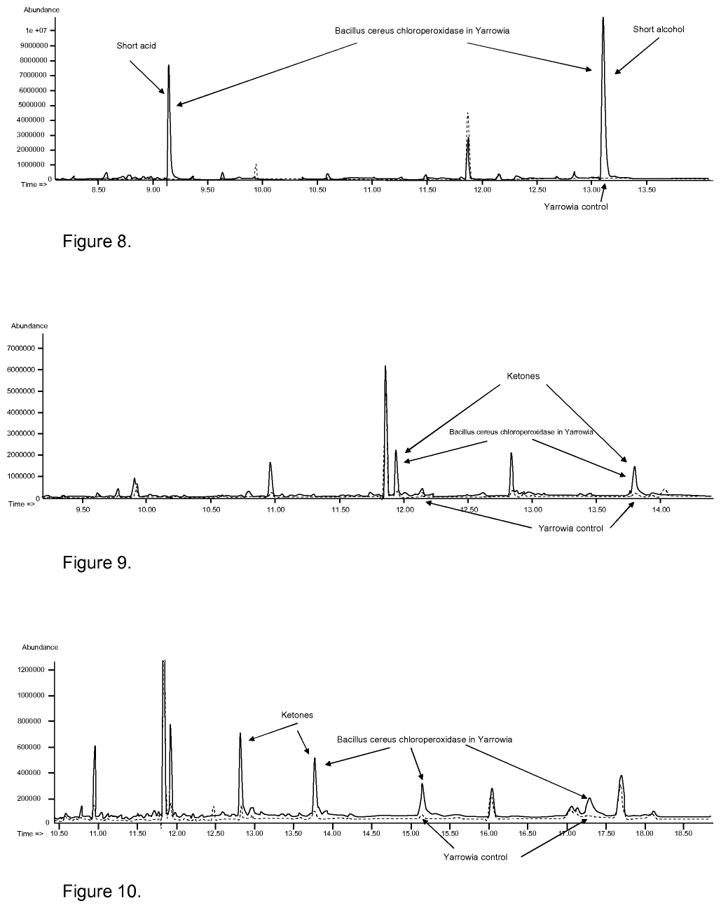

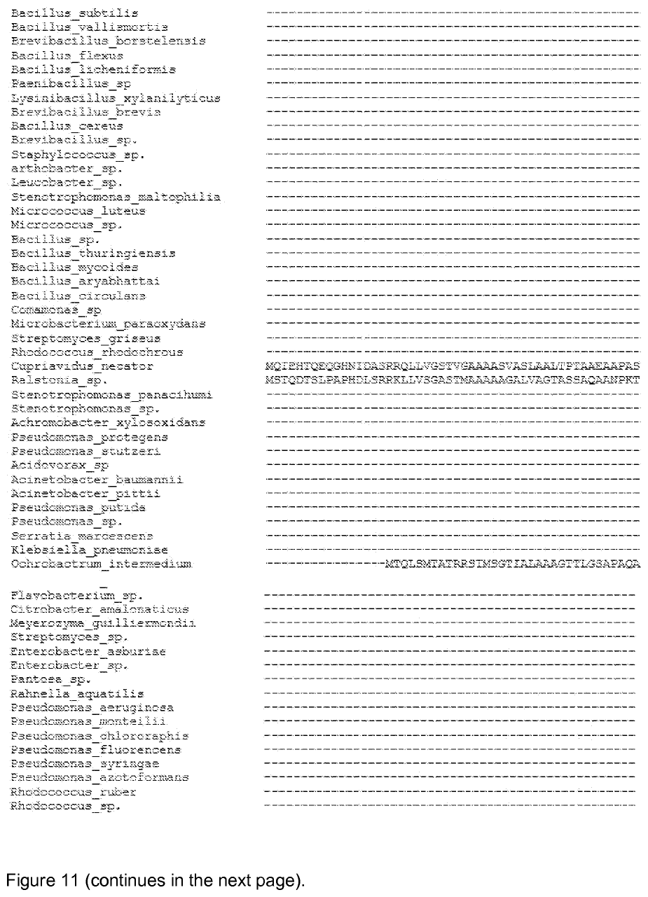

The gene encoding Bacillus cereus chloroperoxidase (SEQ ID NO: 2) amino acid was commercially (Genscript) synthetized with codon optimization for expression in Yarrowia lipolytica cells (SEQ ID NO: 17). PacI and BglII restriction sites were included at 5′ and 3′ ends of construct for restriction digestion cloning. The constructs were cloned into Yarrowia lipolytica integration cassette plasmid B11157 digested with PacI and BclI. B11157 plasmid contains flanks to ANT1 gene and SES promoter (SES promoter described in Rantasalo et al 2018. Nucleic Acids Research, Volume 46, Issue 12 18, October 2018, Page e111, https://doi.org/10.1093/nar/gky558). The resulting plasmid was named as pPB072-1 (FIG. 7). NotI digested integration fragment was transformed into VTT-C-00365 Yarrowia lipolytica strain (VTTCC) with Frozen-EZ yeast transformation kit. After transformation single colonies were cultivated in 20 ml of YPD medium (20 g Bacto peptone, 10 g yeast extract, 20 g glucose per litre) in 100 ml shaken flasks. Wild type Yarrowia lipolytica VTT-C-00365 were cultivated as a control. After 2 days incubation at +30° C. with 200 rpm shaking cultures were centrifuged (3184 g, 10 min RT) and supernatant and pellet samples were separated. Pellet samples were homogenies as follows: Pellet was suspended into 0.5 ml of 100 mM sodium phosphate buffer, pH 7.0 and homogenised with glass beads with Precellys homogenizator. After homogenisation samples were centrifuged (20817 g, 27 min at +4° C.) and cell extracts were collected.

Enzyme activity measurement with cell extract was carried out as follows: 50 μl of cell extract was incubated in 1 ml of 150 mM Mcllvaine pH 3.0 (polyethylene) or pH 6.0 (polypropylene) with 0.2 ml of 17 mM H₂O₂ and PE powder (˜4000 Da, Sigma-Aldrich) or polypropylene powder (Licocene PP 6102 Fine grain, Clariant) at +30° C. for 123 hours. After enzyme reaction GCMS was run from liquid phase as described in Example 3. In GCMS analysis several peaks could be detected with Y. lipolytica strains expressing B. cereus chloroperoxidase with polypropylene (FIG. 8) which could not be detected with control samples (wild type C-00365 Yarrowia lipolytica). With polyethylene several ketone peaks could be detected which were hardly detectable with control sample (FIG. 9). These detected peaks were alkane like compounds like seen in Example 2 (FIG. 1) with B. cereus chloroperoxidase expressed in E. coli. This result indicates that B. cereus chloroperoxidase can be expressed functionally in Y. lipolytica.

Enzyme activity measurement without hydrogen peroxide with supernatant sample was carried out as follows: 100 μl of supernatant sample was incubated in 1 ml of 150 mM Mcllvaine pH 6.0 with PE powder (˜4000 Da, Sigma-Aldrich) at +28° C. for 112 hours. After enzyme reaction GCMS was run from liquid phase as described in Example 3. In GCMS analysis several ketone peaks and alkane like compound peaks could be detected with Y. lipolytica strains expressing B. cereus chloroperoxidase with polyethylene (FIG. 10) which could be hardly detected with control sample (wild type C-00365 Yarrowia lipolytica).

### Example 5. Characterisation of Amino Acid Sequence Motifs of Polyethylene Degrading Chloroperoxidases

A sequence search based on HMMER (Robert D. Finn, Jody Clements, Sean R. Eddy (2011) HMMER web server: interactive sequence similarity searching. Nucleic Acids Research, Volume 39, Issue suppl_2, 1 Jul. 2011, Pages W29-W37, https://doi.org/10.1093/nar/gkr367) was done using the amino acid sequences of SEQ ID NOs: 2, 4, 6 and 8. The HMMER search was carried out against the Uni-ProtKB/TrEMBL. The results were filtered based on the e-value. Several hundreds of sequences were identified. Among these sequences amino acid sequences originating from species which have been shown to degrade polyethylene were collected (SEQ ID Nos: 2, 4, 6, 18-70) and used in multiple sequence alignment carried out with CLUSTAW (https://www.genome.jp/tools-bin/clustalw) with default parameters.

In the alignment several consensus amino acids could be detected (based on Bacillus cereus amino acid position): His29, Gly30, Gln41, Arg51, Asp56, Arg58, Gly61, Ser63, Gly69, Asp78, Leu86, Ser97, Gly99, Pro135, Gly218, Asp221, His249 (see FIG. 11).

To confirm the existence and position of consensus amino acids in a specific enzyme corresponding amino acid sequence was compared to B. cereus chloroperoxidase (SEQ ID NO: 2) by carrying out pairwise alignment with ClustalW default parameters by using Geneious 10.2.6 programme. In FIG. 12 is an example of pairwise alignment between Streptomyces griseus chloroperoxidase (SEQ ID NO: 68) and B. cereus chloroperoxidase (SEQ ID NO: 2). Even these amino acid sequences have only 28.9% identity between each other abovementioned consensus amino acids could be detected and their position in Streptomyces griseus amino acid sequence identified (mark in bold).

### Example 6. Identification of Filamentous Fungi Polyolefin Degrading Chloroperoxidases

Polyolefin degrading chloroperoxidases from filamentous fungi were searched by carrying out Blast search in different genome databases containing specific filamentous fungi genomes. Blast search with default parameters against Bacillus cereus chloroperoxidase amino acid sequence (SEQ ID NO: 2) were carried out in Aspergillus genome database (www.aspqd.org), JGI Mycocosm database (https://mycosom.jgi.doe.gov) or NCBI genome database (https://www.ncbi.nlm.nih.gov/genome/) by selecting specific filamentous fungi species. Amino acid sequences found in Blast searches (SEQ ID Nos: 71-89) were compared to B. cereus chloroperoxidase sequence (SEQ ID NO: 2) with multiple sequence alignment out with CLUSTAW carried (https://www.genome.jp/tools-bin/clustalw) with default parameters.

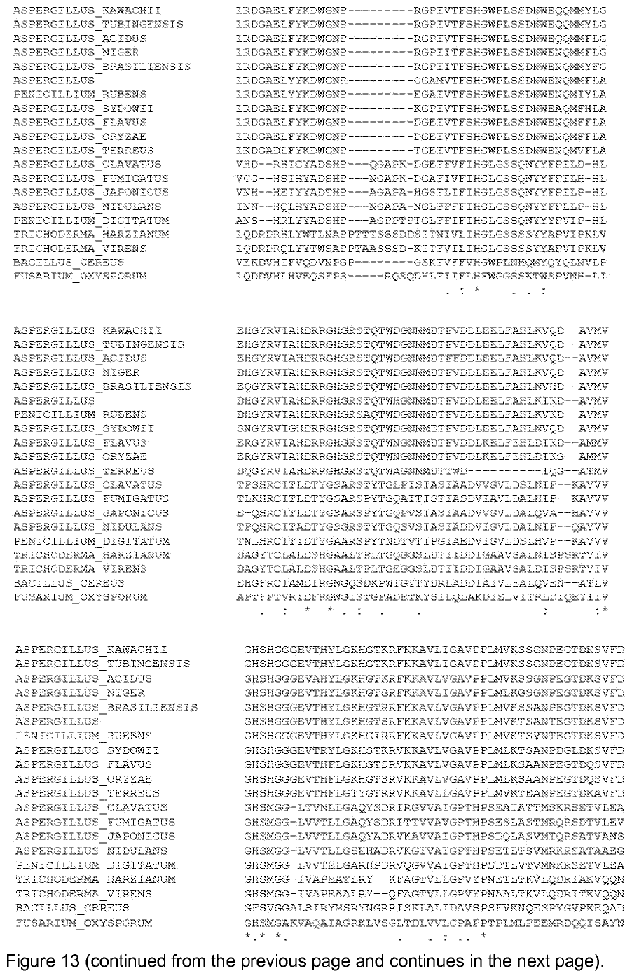

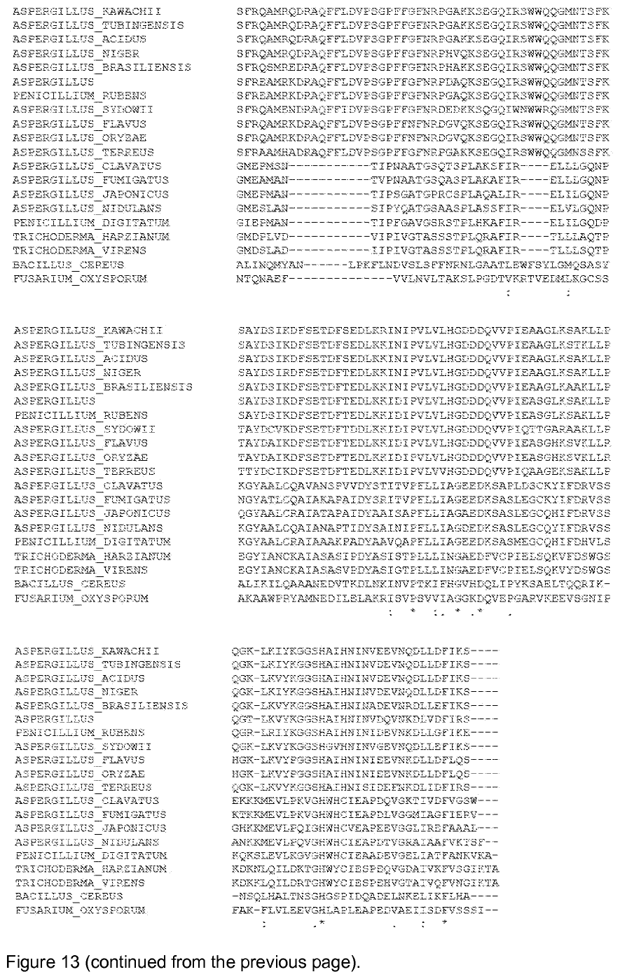

In the alignment several consensus amino acids could be detected (based on Bacillus cereus amino acid position): His29, Asp56, Gly59, Val94, Gly95, Ser97, Gly99, Pro126, Pro212, Gly218, Asp221, His249, Phe266 (see FIG. 13).

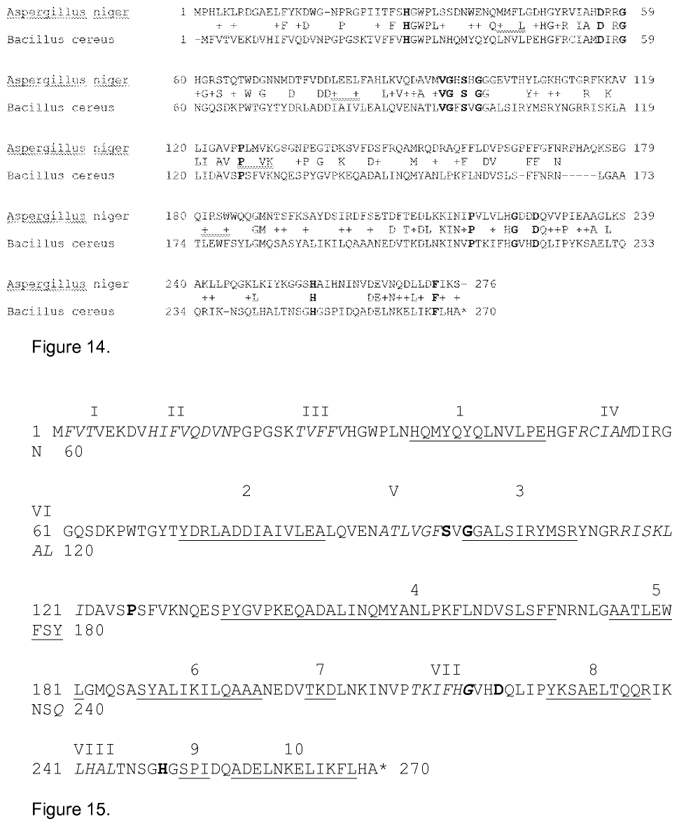

To confirm the existence and position of consensus amino acids in a specific enzyme corresponding amino acid sequence was compared to B. cereus chloroperoxidase (SEQ ID NO: 2) by carrying out pairwise alignment with ClustalW default parameters by using Geneious 10.2.6 programme. In FIG. 14 is an example of pairwise alignment between Aspergillus niger chloroperoxidase (SEQ ID NO: 79) and B. cereus chloroperoxidase (SEQ ID NO: 2). Even these amino acid sequences have only 28% identity between each other abovementioned consensus amino acids could be detected and their position in Aspergillus niger amino acid sequence identified (mark in bold).

### Example 7. Localising Consensus Amino Acids into Enzymes 2D and 3D Structure

Two-dimensional and 3D structures of Bacillus cereus chloroperoxidase (SEQ ID N:O 2) were constructed with Phyre2 protein homology/analogy recognition engine V 2.0 (http://www.sbq.bio.ic.ac.uk/phyre2/html/page.cgi?id=index) with default parameters. The predicted alfa helixes and beta sheets were localised together with identified critical amino acids for enzyme activity as shown in FIG. 15. The critical amino acids for enzyme activity were identified in predicted 3D structure amongst consensus amino acids from Example 5 and 6. These amino acids were in loop area (Ser97, Pro126, Gly218, Asp221 and His249) and in alfa helix 3 (Gly99).

### Example 8. Clostridium sp. Alfa-Beta Hydrolase, Sporomusa Sphaeroides Arylesterase, Microbacterium sp. Bromoperoxidase, Alkalihalobacillus Okhensis Esterase, Paenibacillus sp. Hydrolase and Clostridium carnis Prolyl Iminopeptidase Degrade Polyethylene

The gene encoding Clostridium sp. alfa-beta hydrolase DCM59_17820 (SEQ ID NO: 90) amino acid sequence was commercially (GenScript, US) synthetized with codon optimization for expression in E. coli cells (SEQ ID NO: 91). The insert bearing the coding gene was cleaved from pUC57 vector with FastDigest™ XbaI and HindIII (Thermo Fisher Scientific, US) enzymes. The fragment was ligated with T4 DNA ligase (NEB, US) into pBAT4 vector previously digested with the same enzymes and transformed into E. coli TOP10 competent cells (Thermo Fisher Scientific, US). The resulting plasmid pPB168-2 was then transformed and expressed in E. coli SHuffle® T7 Express cells (NEB, US) as described in Example 1.

The gene encoding Sporomusa sphaeroides arylesterase SPSPH_10950 (SEQ ID NO: 92) amino acid sequence was commercially (GenScript, US) synthetized with codon optimization for expression in E. coli cells (SEQ ID NO: 93). The insert bearing the coding gene was cleaved from pUC57 vector with FastDigest™ XbaI and HindIII (Thermo Fisher Scientific, US) enzymes. The fragment was ligated with T4 DNA ligase (NEB, US) into pBAT4 vector previously digested with the same enzymes and transformed into E. coli TOP10 competent cells (Thermo Fisher Scientific, US). The resulting plasmid pPB169-2 was then transformed and expressed in E. coli SHuffle® T7 Express cells (NEB, US) as described in Example 1.

The gene encoding Microbacterium sp. bromoperoxidase DBR36_08250 (SEQ ID NO: 94) amino acid sequence was commercially (GenScript, US) synthetized with codon optimization for expression in E. coli cells (SEQ ID NO: 95). The insert bearing the coding gene was cleaved from pUC57 vector with FastDigest™ XbaI and HindIII (Thermo Fisher Scientific, US) enzymes. The fragment was ligated with T4 DNA ligase (NEB, US) into pBAT4 vector previously digested with the same enzymes and transformed into E. coli TOP10 competent cells (Thermo Fisher Scientific, US). The resulting plasmid pPB170-2 was then transformed and expressed in E. coli SHuffle® T7 Express cells (NEB, US) as described in Example 1.

The gene encoding Alkalihalobacillus okhensis esterase LQ50_02985 (SEQ ID NO: 96) amino acid sequence was commercially (GenScript, US) synthetized with codon optimization for expression in E. coli cells (SEQ ID NO: 97). The insert bearing the coding gene was cleaved from pUC57 vector with FastDigest™ XbaI and HindIII (Thermo Fisher Scientific, US) enzymes. The fragment was ligated with T4 DNA ligase (NEB, US) into pBAT4 vector previously digested with the same enzymes and transformed into E. coli TOP10 competent cells (Thermo Fisher Scientific, US). The resulting plasmid pPB171-2 was then transformed and expressed in E. coli SHuffle® T7 Express cells (NEB, US) as described in Example 1.

The gene encoding Paenibacillus sp. hydrolase C161_02220 (SEQ ID NO: 98) amino acid sequence was commercially (GenScript, US) synthetized with codon optimization for expression in E. coli cells (SEQ ID NO: 99). The insert bearing the coding gene was cleaved from pUC57 vector with FastDigest™ XbaI and HindIII (Thermo Fisher Scientific, US) enzymes. The fragment was ligated with T4 DNA ligase (NEB, US) into pBAT4 vector previously digested with the same enzymes and transformed into E. coli TOP10 competent cells (Thermo Fisher Scientific, US). The resulting plasmid pPB172-2 was then transformed and expressed in E. coli SHuffle® T7 Express cells (NEB, US) as described in Example 1.

The gene encoding Clostridium carnis prolyl iminopeptidase NCTC10913_04642 (SEQ ID NO: 100) amino acid sequence was commercially (GenScript, US) synthetized with codon optimization for expression in E. coli cells (SEQ ID NO: 101). The insert bearing the coding gene was cleaved from pUC57 vector with FastDigest™ XbaI and HindIII (Thermo Fisher Scientific, US) enzymes. The fragment was ligated with T4 DNA ligase (NEB, US) into pBAT4 vector previously digested with the same enzymes and transformed into E. coli TOP10 competent cells (Thermo Fisher Scientific, US). The resulting plasmid pPB173-2 was then transformed and expressed in E. coli SHuffle® T7 Express cells (NEB, US) as described in Example 1.

E. coli cultivations with pPB168-2, 169-2, 170-2, 171-2 and 173-2 expressing E. coli SHuffle® T7 Express cells were carried out as described in Example 1. Supernatant samples were collected and used in enzyme assays. Supernatant sample from E. coli cultivation with pBAT4 plasmid expressing E. coli was used as a negative control.

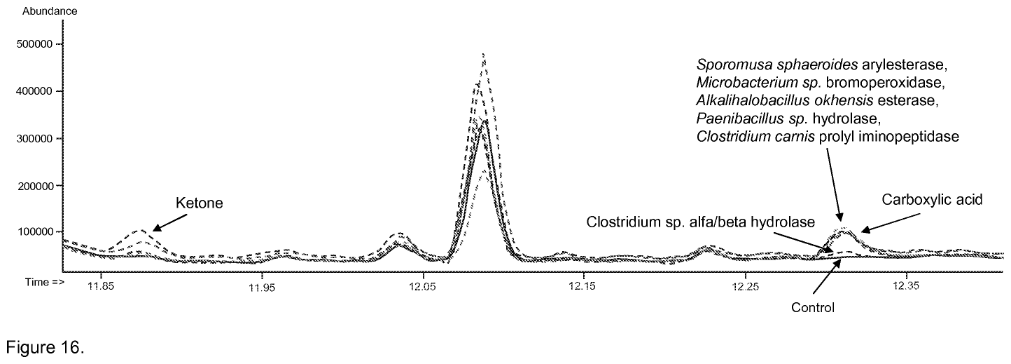

Enzyme assay with polyethylene with Clostridium sp. alfa-beta hydrolase, Sporomusa sphaeroides arylesterase, Microbacterium sp. bromoperoxidase, Alkalihalobacillus okhensis esterase, Paenibacillus sp. hydrolase and Clostridium carnis prolyl iminopeptidase enzymes were carried out with supernatant samples as follows: 1 ml of 50 mM HEPES pH 8.0 with polyethylene powder (average MW˜4000 dalton, Sigma-Aldrich) was incubated with 100 μl of E. coli supernatant samples at +30° C. for 140 hours with 600 rpm shaking. As a control supernatant from the culture with E. coli strain having empty plasmid (pBAT4) was used. After incubation GC-MS run was carried out with liquid fraction as described in Example 3. In GC-MS analysis carboxylic acid and ketone peaks were detected with enzyme samples which were missing from the control sample (FIG. 16) indicating polyethylene degradation.

### Example 9. Sporomusa Sphaeroides Arylesterase Degrades Polyethylene

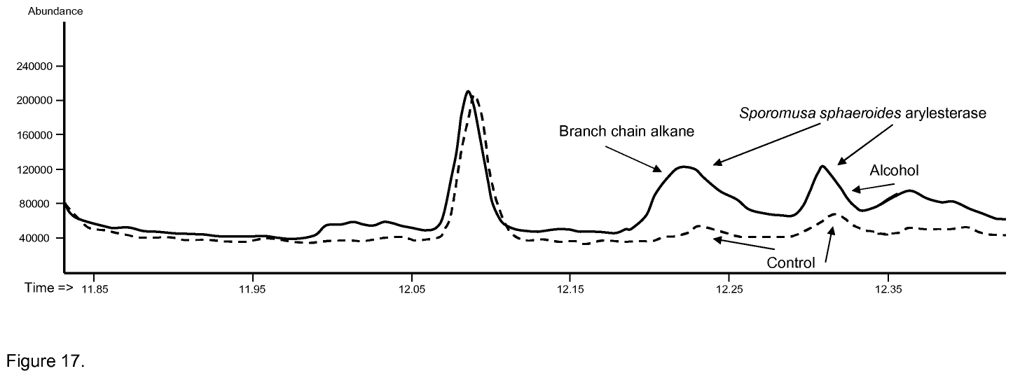

Enzyme assay with polypropylene with Sporomusa sphaeroides arylesterase enzyme was carried out with supernatant samples as follows: 1.2 ml of 150 mM Mcllvaine buffer pH 6.0 and 17 mM H₂O₂ with polypropylene powder (Licocene PP 6102 Fine grain, Clariant) was incubated with 100 μl of E. coli supernatant sample from Example 8 at +30° C. for 140 hours with 600 rpm shaking. As a control supernatant from the culture with E. coli strain having empty plasmid (pBAT4) was used. After incubation GC-MS run was carried out with liquid fraction as described in Example 3. In GC-MS analysis branch chain alkane and alcohol peaks were detected with enzyme sample which were missing in the control sample (FIG. 17) indicating degradation of polypropylene.

### Example 10. Hydrogen Peroxide Producing Superoxide Dismutase Enhanced Polyethylene Degradation with Paenibacillus sp. Hydrolase

The gene encoding Bacillus licheniformis superoxide dismutase (SEQ ID NO: 102) amino acid was cloned from genomic Bacillus licheniformis DNA by PCR by using oligonucleotides oPlastBug-242 (TAGAAATAATTTTGTTTAACTTTAAGAAGGAGATATATCCATGGCTTACAAACT TCCAGAATTACCTTATGCT, SEQ ID NO: 104) and oPlastBug-243 (CAAGCTGGGATTTAGGTGACACTATAGAATACTCAAGCTTTTATTTTGCTTCG CTGTAAAGGCGTGC, SEQ ID NO: 105). The resulting DNA fragment containing coding region of the gene (SEQ ID NO: 103) was cloned into NcoI and HindIII digested Escherichia coli expression vector pBAT4 with Gibson assembly resulting in plasmid pPB095-1 and expressed in E. coli strain Shuffle T7 Express (New England Biolabs). Supernatant sample of enzyme was produced as described in Example 1.

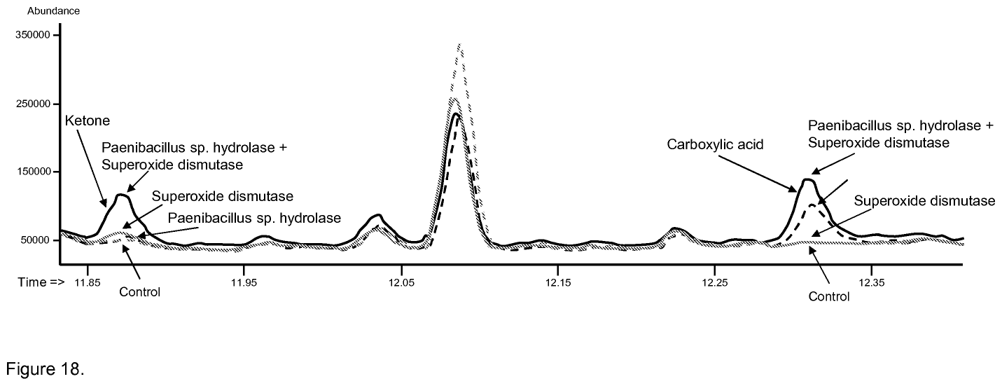

Enzyme assays with Paenibacillus sp. hydrolase and Bacillus licheniformis superoxide dismutase were carried as follows: 1 ml of 50 mM HEPES pH 8.0 with polyethylene powder (average MW˜4000 dalton, Sigma-Aldrich) was incubated with 100 μl of Paenibacillus sp. hydrolase E. coli supernatant sample with and without 25 μl of Bacillus licheniformis superoxide dismutase E. coli supernatant sample at +30° C. for 140 hours with 600 rpm shaking. As a controls supernatant from the culture with E. coli strain having empty plasmid (pBAT4) was used with and without superoxide dismutase. After incubation GC-MS run was carried out with liquid fraction as described in Example 3. In GC-MS analysis higher ketone and carboxylic acid peaks could be detected with the sample having both hydrolase and superoxide dismutase than with the samples having hydrolase or superoxide alone. In all cases enzyme samples had higher peaks than the control sample (FIG. 18).

1. A method of degrading a polyolefin, said method comprising
providing a material comprising a polyolefin and an enzyme or a fragment thereof capable of degrading the polyolefin, and
allowing said enzyme or fragment thereof to degrade the polyolefin,
wherein the enzyme or fragment thereof comprises a catalytic triad Ser-Asp-His corresponding to amino acids Ser97, Asp221 and His249 presented in SEQ ID NO: 2, and optionally one or more amino acids Gly99, Pro126 and Gly218 corresponding to the amino acids presented in SEQ ID NO: 2, and wherein the enzyme is optionally genetically modified.

2. An isolated enzyme or a fragment thereof comprising a catalytic triad Ser-Asp-His corresponding to amino acids Ser97, Asp221 and His249 presented in SEQ ID NO: 2, and optionally one or more amino acids Gly99, Pro126 and Gly218 corresponding to the amino acids presented in SEQ ID NO: 2, and wherein said enzyme or fragment is capable of degrading a polyolefin, wherein the enzyme is optionally genetically modified.

3. A micro-organism or a host cell comprising an enzyme or a fragment thereof comprising a catalytic triad Ser-Asp-His corresponding to amino acids Ser97, Asp221 and His249 presented in SEQ ID NO: 2, and optionally one or more amino acids Gly99, Pro126 and Gly218 corresponding to the amino acids presented in SEQ ID NO: 2, and wherein said enzyme or fragment is capable of degrading a polyolefin, wherein the enzyme is optionally genetically modified.

4. (canceled)

5. The method of claim 1, wherein the enzyme or a fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, or 8, and the enzyme optionally originates from or is an enzyme of a bacterium or fungus selected from the group comprising or consisting of Bacillus, Paenibacillus, Achromobacter, Acinetobacter, Acidovorax, Alcanivorax, Aneurinibacillus, Arthrobacter, Aspergillus, Brevibacillus, Chaetomium, Chitinophaga, Citrobacter, Cladosporium, Cupriavidus, Comamonas, Cordyceps, Cupriavidus, Delftia, Engyodontium, Enterobacter, Flavobacterium, Fusarium, Hyphomicrobium, Hypocrea, Klebsiella, Kocuria, Leucobacter, Lulwoana, Lysinibacillus, Macrococcus, Methylobacterium, Methylocella, Meyerozyma, Microbacterium, Micrococcus, Moraxella, Mortierella, Mucor, Nesiotobacter, Nocardia, Ochrobactrum, Oscillatoria, Pantoea, Paracoccus, Penicillium, Phanerochaete, Phormidium, Pleurotus, Pseudomonas, Rahnella, Ralstonia, Rhizobium, Rhodococcus, Sarocladium, Serratia, Staphylococcus, Stenotrophomonas, Streptococcus, Streptomyces, Trichoderma, Vibrio, Virgibacillus and Xanthobacter; or the enzyme is an enzyme of a bacterium or fungus selected from the group comprising or consisting of Achromobacter xylosoxidans, Acidovorax sp., Acinetobacter sp., Acinetobacter baumannii, Acinetobacter pittii, Alcanivorax borkumensis, Aneurinibacillus aneurinilyticus, Arthrobacter sp, Aspergillus acidus, Aspergillus awamori, Aspergillus brasiliensis, Aspergillus clavatus, Aspergillus flavus, Aspergillus foetidus, Aspergillus fumigatus, Aspergillus glaucus, Aspergillus japonicus, Aspergillus kawachii, Aspergillus nidulans, Aspergillus niger, Aspergillus nomius, Aspergillus oryzae, Aspergillus sp. Aspergillus sydowii, Aspergillus terreus, Aspergillus tubingensis, Aspergillus wentii, Bacillus amyloliquefaciens, Bacillus aryabhattai, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus gottheilii, Bacillus vallismortis, Bacillus vietnamensis, Brevibacillus sp., Brevibacillus brevis, Brevibacillus borstelensis, Brevibacillus agri, Brevibacillus parabrevis, Chaetomiumsp., Chitinophaga sp., Citrobacter amalonaticus, Cladosporium cladosporioides, Comamonas sp., Cordyceps confragosa, Cupriavidus necator, Delftia sp., Delftia tsuruhatensis, Engyodontium album, Enterobacter sp., Enterobacter asburiare, Flavobacterium sp., Flavobacterium petrolei, Flavobacterium pectinovorum, Flavobacterium aquicola, Fusarium redolens, Fusarium oxysporum, Fusarium solani, Fusarium sp., Hyphomicrobium sp., Hypocrea sp., Klebsiella pneumoniae, Kocuria palustris, Leucobacter sp., Lulwoana uniseptata, Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Meyerozyma guilliermondii, Microbacterium sp., Microbacterium paraoxydans, Micrococcus sp., Micrococcus luteus, Micrococcus lylae, Moraxella sp., Mortierella alpina, Mucor circinelloides, Mucor sp., Nesiotobacter exalbescens, Nocardia asteroides, Ochrobactrum intermedium, Ochrobactrum oryzae, Oscillatoria subbrevis, Paenibacillus sp., Paenibacillus odorifer, Paenibacillus macerans, Pantoea sp., Penicillium digitatum, Penicillium chrysogenum, Penicillium glabrum, Penicillium oxalicum, Penicillium rubens, Penicillium simplicissimum, Penicillium sp., Paracoccus yeei, Phanerochaete chrysosporium, Phormidium lucidum, Pleurotus ostreatus, Pseudomonas aeruginosa, Pseudomonas azotoformans, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus gingshengii, Rhodococcus erythropolis, Rhodococcus rhodochrous, Rhodococcus ruber, Rhodococcus sp., Sarocladium kiliense, Serratia marcescens, Sphingobacterium multivorum, Staphylococcus sp., Staphylococcus epidermidis, Staphylococcus cohnii, Staphylococcus xylosus, Stenotrophomonas humi, Stenotrophomonas maltophilia, Stenotrophomonas panacihumi, Stenotrophomonas sp., Streptococcus sp., Streptomyces albogriseolus, Streptomyces badius, Streptomyces griseus, Streptomyces sp., Streptomyces viridosporus, Trichoderma harzianum, Trichoderma virens, Trichoderma viride, Vibrio alginolyticus, Vibrio parahaemolyticus, Virgibacillus halodenitrificans, Xanthobacter autotrophicus, and Xanthobacter tagetidis.

6. The isolated enzyme or fragment thereof of claim 2, wherein the enzyme or the fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, or 8, and wherein the enzyme originates from or is an enzyme of a bacterium or fungus selected from the group comprising or consisting of Bacillus, Paenibacillus, Achromobacter, Acinetobacter, Acidovorax, Alcanivorax, Aneurinibacillus, Arthrobacter, Aspergillus, Brevibacillus, Chaetomium, Chitinophaga, Citrobacter, Cladosporium, Cupriavidus, Comamonas, Cordyceps, Cupriavidus, Delftia, Engyodontium, Enterobacter, Flavobacterium, Fusarium, Hyphomicrobium, Hypocrea, Klebsiella, Kocuria, Leucobacter, Lulwoana, Lysinibacillus, Macrococcus, Methylobacterium, Methylocella, Meyerozyma, Microbacterium, Micrococcus, Moraxella, Mortierella, Mucor, Nesiotobacter, Nocardia, Ochrobactrum, Oscillatoria, Pantoea, Paracoccus, Penicillium, Phanerochaete, Phormidium, Pleurotus, Pseudomonas, Rahnella, Ralstonia, Rhizobium, Rhodococcus, Sarocladium, Serratia, Staphylococcus, Stenotrophomonas, Streptococcus, Streptomyces, Trichoderma, Vibrio, Virgibacillus and Xanthobacter; or
the enzyme is an enzyme of a bacterium or fungus selected from the group comprising or consisting of Achromobacter xylosoxidans, Acidovorax sp., Acinetobacter sp., Acinetobacter baumannii, Acinetobacter pittii, Alcanivorax borkumensis, Aneurinibacillus aneurinilyticus, Arthrobacter sp, Aspergillus acidus, Aspergillus awamori, Aspergillus brasiliensis, Aspergillus clavatus, Aspergillus flavus, Aspergillus foetidus, Aspergillus fumigatus, Aspergillus glaucus, Aspergillus japonicus, Aspergillus kawachii, Aspergillus nidulans, Aspergillus niger, Aspergillus nomius, Aspergillus oryzae, Aspergillus sp. Aspergillus sydowii, Aspergillus terreus, Aspergillus tubingensis, Aspergillus wentii, Bacillus amyloliquefaciens, Bacillus aryabhattai, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus gottheilii, Bacillus vallismortis, Bacillus vietnamensis, Brevibacillus sp., Brevibacillus brevis, Brevibacillus borstelensis, Brevibacillus agri, Brevibacillus parabrevis, Chaetomium sp., Chitinophaga sp., Citrobacter amalonaticus, Cladosporium cladosporioides, Comamonas sp., Cordyceps confragosa, Cupriavidus necator, Delftia sp., Delftia tsuruhatensis, Engyodontium album, Enterobacter sp., Enterobacter asburiare, Flavobacterium sp., Flavobacterium petrolei, Flavobacterium pectinovorum, Flavobacterium aquicola, Fusarium redolens, Fusarium oxysporum, Fusarium solani, Fusarium sp., Hyphomicrobium sp., Hypocrea sp., Klebsiella pneumoniae, Kocuria palustris, Leucobacter sp., Lulwoana uniseptata, Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Meyerozyma guilliermondii, Microbacterium sp., Microbacterium paraoxydans, Micrococcus sp., Micrococcus luteus, Micrococcus lylae, Moraxella sp., Mortierella alpina, Mucor circinelloides, Mucor sp., Nesiotobacter exalbescens, Nocardia asteroides, Ochrobactrum intermedium, Ochrobactrum oryzae, Oscillatoria subbrevis, Paenibacillus sp., Paenibacillus odorifer, Paenibacillus macerans, Pantoea sp., Penicillium digitatum, Penicillium chrysogenum, Penicillium glabrum, Penicillium oxalicum, Penicillium rubens, Penicillium simplicissimum, Penicillium sp., Paracoccus yeei, Phanerochaete chrysosporium, Phormidium lucidum, Pleurotus ostreatus, Pseudomonas aeruginosa, Pseudomonas azotoformans, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus gingshengii, Rhodococcus erythropolis, Rhodococcus rhodochrous, Rhodococcus ruber, Rhodococcus sp., Sarocladium kiliense, Serratia marcescens, Sphingobacterium multivorum, Staphylococcus sp., Staphylococcus epidermidis, Staphylococcus cohnii, Staphylococcus xylosus, Stenotrophomonas humi, Stenotrophomonas maltophilia, Stenotrophomonas panacihumi, Stenotrophomonas sp., Streptococcus sp., Streptomyces albogriseolus, Streptomyces badius, Streptomyces griseus, Streptomyces sp., Streptomyces viridosporus, Trichoderma harzianum, Trichoderma virens, Trichoderma viride, Vibrio alginolyticus, Vibrio parahaemolyticus, Virgibacillus halodenitrificans, Xanthobacter autotrophicus, and Xanthobacter tagetidis.

7. The method of claim 1, wherein the enzyme is selected from the group comprising or consisting of a hydrolase, perhydrolase, esterase, aryl esterase, carboxylesterase, enol-lactonase, aminopeptidase, serine protease, lipase, epoxide hydrolase, C—C hydrolase, dehalogenase, lyase, dioxygenase, peroxidase, chloroperoxidase, bromoperoxidase, haloperoxidase and non-heme chloroperoxidase; and/or
the enzyme comprises hydrolase, perhydrolase, esterase, aryl esterase, carboxylesterase, enol-lactonase, aminopeptidase, serine protease, lipase, epoxide hydrolase, C—C hydrolase, dehalogenase, lyase, dioxygenase, peroxidase, chloroperoxidase, bromoperoxidase, haloperoxidase or non-heme chloroperoxidase activity, or any combination thereof.

8. The method of claim 1, wherein the polyolefin is polyethylene (PE), cross-linked polyethylene (PEX or XLPE), ultra-high molecular weight polyethylene (UHMWPE), high-density polyethylene (HDPE), medium-density polyethylene (MDPE), linear low-density polyethylene (LLDPE), low density polyethylene (LDPE), very low density polyethylene (VLDPE), polypropylene (PP), polymethylpentene (PMP), polybutene-1 (PB-1), polyisobutylene (PIB), or any combination thereof; or wherein the enzyme or a fragment thereof is capable of degrading a polyethylene and/or a polypropylene.

9. The isolated enzyme or fragment thereof according to claim 2, wherein the isolated enzyme or fragment thereof is capable of degrading the polyolefin in the presence or absence of hydrogen peroxide at a temperature of at least 20° C., at least 25° C., at least 30° C., at least 37° C. or at a temperature of 30-95° C.

10. The micro-organism or host cell according to claim 3, wherein the micro-organism or host cell is capable of degrading the polyolefin in the presence or absence of hydrogen peroxide at a temperature of at least 20° C., at least 25° C., at least 30° C., at least 37° C. or at a temperature of 30-95° C.

11. The isolated enzyme or fragment thereof according to claim 9, wherein the isolated enzyme or fragment thereof is capable of degrading the polyolefin in the presence of hydrogen peroxide and the hydrogen peroxide is produced by another enzyme, fragment, micro-organism or host cell, such as a superoxide dismutase or a fragment thereof, or a micro-organism or host cell comprising said superoxide dismutase.

12. The method of claim 1, wherein at least one or more degradation products is selected from the group consisting of an alkane, alkene, alkyne, cycloalkane, alkadiene, ketone, fatty acid, alcohol, aldehyde, epoxy, diacid, dione, 2.9-decanedione, 2.11-dodecanedione, 2-decanone, 2-dodecanone, 2-tetradecanone, 2-hexadecanone, 2-heptadecanone and 2-dotriacontanone are obtained or obtainable by the degradation of the polyolefin.

13. (canceled)

14. The method of claim 1, wherein the enzyme or fragment thereof has an increased ability to degrade the polyolefin compared to a corresponding unmodified enzyme or fragment thereof.

15. The method of claim 1, wherein the enzyme or fragment thereof comprises a signal sequence, or does not comprise a detectable signal sequence, and wherein the enzyme or fragment thereof is secreted out of the cell, or is not secreted out of the cell which produces it.

16. The method of claim 1, wherein the enzyme or fragment thereof is encoded by a heterologous polynucleotide sequence and optionally expressed by a micro-organism or host cell.

17. The micro-organism or host cell of claim 3, wherein the micro-organism or host cell is selected from the group consisting of Escherichia, Yarrowia, Pichia, Saccharomyces, Trichoderma, Aspergillus, Bacillus, Myceliophthora, Escherichia coli, Yarrowia lipolytica, Pichia pastoris, Saccharomyces cerevisiae, Trichoderma reesei, Aspergillus nidulans, Aspergillus niger, Aspergillus oryzae, Bacillus licheniformis, Bacillus subtilis, and Myceliophthora thermophila.

18. A polynucleotide encoding the enzyme or fragment thereof of claim 2.

19. An expression vector or plasmid comprising the polynucleotide of claim 18.

20. (canceled)

21. A method of producing the enzyme or fragment thereof of claim 2, wherein a recombinant micro-organism or host cell comprising a polynucleotide encoding the enzyme or fragment thereof is allowed to express said enzyme or fragment thereof.

22. The micro organism or host cell of claim 3, wherein enzyme or a fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, or 8, and wherein the enzyme originates from or is an enzyme of a bacterium or fungus selected from the group comprising or consisting of Bacillus, Paenibacillus, Achromobacter, Acinetobacter, Acidovorax, Alcanivorax, Aneurinibacillus, Arthrobacter, Aspergillus, Brevibacillus, Chaetomium, Chitinophaga, Citrobacter, Cladosporium, Cupriavidus, Comamonas, Cordyceps, Cupriavidus, Delftia, Engyodontium, Enterobacter, Flavobacterium, Fusarium, Hyphomicrobium, Hypocrea, Klebsiella, Kocuria, Leucobacter, Lulwoana, Lysinibacillus, Macrococcus, Methylobacterium, Methylocella, Meyerozyma, Microbacterium, Micrococcus, Moraxella, Mortierella, Mucor, Nesiotobacter, Nocardia, Ochrobactrum, Oscillatoria, Pantoea, Paracoccus, Penicillium, Phanerochaete, Phormidium, Pleurotus, Pseudomonas, Rahnella, Ralstonia, Rhizobium, Rhodococcus, Sarocladium, Serratia, Staphylococcus, Stenotrophomonas, Streptococcus, Streptomyces, Trichoderma, Vibrio, Virgibacillus and Xanthobacter; or
the enzyme is an enzyme of a bacterium or fungus selected from the group comprising or consisting of Achromobacter xylosoxidans, Acidovorax sp., Acinetobacter sp., Acinetobacter baumannii, Acinetobacter pittii, Alcanivorax borkumensis, Aneurinibacillus aneurinilyticus, Arthrobacter sp, Aspergillus acidus, Aspergillus awamori, Aspergillus brasiliensis, Aspergillus clavatus, Aspergillus flavus, Aspergillus foetidus, Aspergillus fumigatus, Aspergillus glaucus, Aspergillus japonicus, Aspergillus kawachii, Aspergillus nidulans, Aspergillus niger, Aspergillus nomius, Aspergillus oryzae, Aspergillus sp. Aspergillus sydowii, Aspergillus terreus, Aspergillus tubingensis, Aspergillus wentii, Bacillus amyloliquefaciens, Bacillus aryabhattai, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus gottheilii, Bacillus vallismortis, Bacillus vietnamensis, Brevibacillus sp., Brevibacillus brevis, Brevibacillus borstelensis, Brevibacillus agri, Brevibacillus parabrevis, Chaetomium sp., Chitinophaga sp., Citrobacter amalonaticus, Cladosporium cladosporioides, Comamonas sp., Cordyceps confragosa, Cupriavidus necator, Delftia sp., Delftia tsuruhatensis, Engyodontium album, Enterobacter sp., Enterobacter asburiare, Flavobacterium sp., Flavobacterium petrolei, Flavobacterium pectinovorum, Flavobacterium aquicola, Fusarium redolens, Fusarium oxysporum, Fusarium solani, Fusarium sp., Hyphomicrobium sp., Hypocrea sp., Klebsiella pneumoniae, Kocuria palustris, Leucobacter sp., Lulwoana uniseptata, Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Meyerozyma guilliermondii, Microbacterium sp., Microbacterium paraoxydans, Micrococcus sp., Micrococcus luteus, Micrococcus Iylae, Moraxella sp., Mortierella alpina, Mucor circinelloides, Mucor sp., Nesiotobacter exalbescens, Nocardia asteroides, Ochrobactrum intermedium, Ochrobactrum oryzae, Oscillatoria subbrevis, Paenibacillus sp., Paenibacillus odorifer, Paenibacillus macerans, Pantoea sp., Penicillium digitatum, Penicillium chrysogenum, Penicillium glabrum, Penicillium oxalicum, Penicillium rubens, Penicillium simplicissimum, Penicillium sp., Paracoccus yeei, Phanerochaete chrysosporium, Phormidium lucidum, Pleurotus ostreatus, Pseudomonas aeruginosa, Pseudomonas azotoformans, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus gingshengii, Rhodococcus erythropolis, Rhodococcus rhodochrous, Rhodococcus ruber, Rhodococcus sp., Sarocladium kiliense, Serratia marcescens, Sphingobacterium multivorum, Staphylococcus sp., Staphylococcus epidermidis, Staphylococcus cohnii, Staphylococcus xylosus, Stenotrophomonas humi, Stenotrophomonas maltophilia, Stenotrophomonas panacihumi, Stenotrophomonas sp., Streptococcus sp., Streptomyces albogriseolus, Streptomyces badius, Streptomyces griseus, Streptomyces sp., Streptomyces viridosporus, Trichoderma harzianum, Trichoderma virens, Trichoderma viride, Vibrio alginolyticus, Vibrio parahaemolyticus, Virgibacillus halodenitrificans, Xanthobacter autotrophicus, and Xanthobacter tagetidis.

23. The enzyme or fragment thereof according to claim 2, wherein the enzyme is selected from the group comprising or consisting of a hydrolase, perhydrolase, esterase, aryl esterase, carboxylesterase, enol-lactonase, aminopeptidase, serine protease, lipase, epoxide hydrolase, C—C hydrolase, dehalogenase, lyase, dioxygenase, peroxidase, chloroperoxidase, bromoperoxidase, haloperoxidase and non-heme chloroperoxidase; and/or
the enzyme comprises hydrolase, perhydrolase, esterase, aryl esterase, carboxylesterase, enol-lactonase, aminopeptidase, serine protease, lipase, epoxide hydrolase, C—C hydrolase, dehalogenase, lyase, dioxygenase, peroxidase, chloroperoxidase, bromoperoxidase, haloperoxidase or non-heme chloroperoxidase activity, or any combination thereof.
